(19) United States
(12) Patent Application Publication
(10) Pub. No.: US 2022/0378700 A1
(43) Pub. Date: Dec. 1, 2022

(54) Lipid Nanoparticles and Formulations Thereof for CAR mRNA Delivery

(71) Applicant: The Trustees of the University of Pennsylvania, Philadelphia, PA (US)

(72) Inventors: Michael Mitchell, Philadelphia, PA (US); Margaret Billingsley, Hockessin, DE (US); Nathan Singh, St. Louis, MO (US); Carl H. June, Merion Station, PA (US)

(21) Appl. No.: 17/769,893

(22) PCT Filed: Oct. 19, 2020
(86) PCT No.: PCT/US20/56255

§ 371 Date: Apr. 18, 2022

Related U.S. Application Data
(60) Provisional application No. 62/916,942, filed on Oct. 18, 2019.

(51) Int. Cl. A61K 9/127; A61K 31/711; C12N 15/87; A61K 39/39

(52) U.S. Cl. CPC A61K 9/1272; A61K 31/711; C12N 15/87; A61K 39/39

(57) ABSTRACT

The present invention relates to lipid nanoparticles (LNP) or compositions thereof for delivery of mRNA molecules encoding CAR, nucleic acid molecule, and/or therapeutic agents to selected targets, such as cells. Thus, in various aspects, the present invention also provides methods of preventing or treating diseases or disorders in a subject in need thereof using the said LNPs or compositions thereof.

### CROSS-REFERENCE TO RELATED APPLICATIONS

This application claims priority to and the benefit of U.S. Provisional Application No. 62/916,942, filed Oct. 18, 2019, the disclosure of which is incorporated herein by reference in its entirety.

### STATEMENT REGARDING FEDERALLY SPONSORED RESEARCH OR DEVELOPMENT

This invention was made with government support under R01-CA-226983 awarded by the National Cancer Institute and under DP2 TR002776 awarded by the National Institutes of Health. The government has certain rights in the invention.

### BACKGROUND OF THE INVENTION

The generation of chimeric antigen receptor (CAR) T cells, an FDA-approved cancer therapy, relies on altering patient T cells to express the cancer-targeting CAR transmembrane protein and reinfusing them back into the patient. To induce CAR expression, the current manufacturing process for clinical application relies on virus-based cell engineering. Using virus to engineer T cells leads to permanent CAR expression on the T cell surfaces. While potent, this can amplify the adverse side effects (i.e., cytokine storm, neurotoxicity) associated with CAR T cell therapy. Further, any manufacturing mistakes are also permanent and can lead to fatal consequences. Thus, clinical researchers have begun investigating mRNA-based CAR T cell production where mRNA encoding for CAR is delivered to T cells. This leads to transiently expressed CAR on T cells that has shown promising results as a means to mitigate unwanted toxic side effects.

However, T cells are distinctly challenging to transfect. Thus, to deliver mRNA to T cells, the most commonly utilized method is electroporation (EP). EP uses electric pulses to open pores in the cell membrane and allow anything in solution with the cell (in this case, mRNA) to enter the cytosol. Though effective at getting mRNA into cells, EP tends to be toxic to T cells, can lead to altered genome expression, and has no potential for in vivo translation.

In 2017, the FDA approved CD19 CAR T cell therapy for the treatment of relapsed or refractory acute lymphoblastic leukemia (ALL) given its ability to induce high rates of durable remission (Liu Y et al., 2017, Drugs of Today, 53:597-608; Maude S L et al., 2014, N Engl J Med., 371:1507-1517). In the same year, the therapy was approved for the treatment of relapsed or refractory large B cell lymphoma as it was also shown to induce complete remission in the majority of patients (Bouchkouj N et al., 2019, Clin Cancer Res, 25:1702-1708; Yip A et al., 2018, Nat Rev Drug Discov, 17:161). Since these successes, many explorations into CAR T cell therapy for the treatment of other cancers, including chronic lymphoblastic leukemia (Porter D L et al., 2011, N Engl J Med, 365:725-733), glioblastoma (Brown C E et al., 2016, N Engl J Med, 375:2561-2569), and refractory multiple myeloma (Berdeja J G et al., 2017, J Clin Oncol, 35), have begun but have yet to receive FDA approval. The development of these autologous therapies relies on ex vivo cell engineering to produce CAR T cells. To produce this form of cancer immunotherapy, patient T cells are harvested, modified to express CD19 specific CAR, and reinfused into the patient. The transmembrane CAR construct allows T cells to target and bind cancerous B cells to induce apoptosis and thus, eradicate the cancer using the patient's own immune system (Benmebarek M et al., 2019, Int J Mol Sci, 20:1283).

Though this process yields potent CAR T cells that induce durable remission in a majority of patients (Maude S L et al., 2014, N Engl J Med, 371:1507-1517; Bouchkouj N et al., 2019, Clin Cancer Res, 25:1702-1708; Yip A et al., 2018, Nat Rev Drug Discov; 17:161), the therapy has serious adverse effects attributed to patient immune response and potential risks associated with viral transduction and production errors (Bouchkouj N et al., 2019, Clin Cancer Res, 25:1702-1708; June C H et al., 2015, Sci Transl Med, 7; June C H et al., 2014, Cancer Immunol Immunother, 63:969-975; Fesnak A D et al., 2016, Nat Rev Cancer, 16: 566-581; Vornittag P et al., 2018, Curr Opin Biotechnol, 53:164-181; Ruella M et al., 2018, Nat Med., 24:1499-1503). Immediate reactions, which have been found to occur in nearly 70% of adult patients receiving the therapy (Hartsell A et al., 2019, Biol Blood Marrow Transplant., 25:S336-S337), include macrophage activation syndrome, neurotoxicity, and cytokine storm (Bouchkouj N et al., 2019, Clin Cancer Res, 25: 1702-1708; June C H et al., 2014, Cancer Immunol Immunother, 63:969-975; Zheng P P et al., 2019, Trends Immunol, 40:274-278). While some of the initial adverse events may be mitigated with anti-IL-6 receptor antibodies (Maude S L et al., 2014, N Engl J Med, 371:1507-1517), the long term effects can be equally as severe. After targeting cancerous B cells, persisting CD19 specific CAR T cells may lead to the elimination of all CD19 positive cells, which results in B cell aplasia and hypogammaglobulinemia (Porter D L et al., 2011, N Engl J Med. 365:725-733; June C H et al., 2014, Cancer Immunol Immunother, 63:969-975; Dotti G et al., 2014, Immunol Rev, 257:107-126; Yang G et al., 2015, Cell, 344:1173-1178). In addition to these toxicity concerns, CAR T cells with continuous expression, including those that are FDA approved, are most commonly produced via viral transduction, which also presents safety concerns over toxic insertional mutagenesis (Porter D L et al., 2011, N Engl J Med, 365:725-733; June C H et al., 2015, Sci Transl Med, 7; Fesnak A D et al., 2016, Nat Rev Cancer, 16:566-581; Vormittag P et al., 2018, Curr Opin Biotechnol, 53:164-181; Dotti G et al., 2014, Immunol Rev; 257:107-126). Further, the permanent alteration can have fatal consequences in instances of production error. In one case, the accidental transduction of a single cancerous B cell during ex vivo CAR T cell engineering led to patient death (Ruella M et al., 2018, Nat Med, 24:1499-1503). In total, these adverse effects highlight the risks associated with this potent therapy and motivate an investigation into improving CAR T cell production methods to generate safer CAR T cells.

One potential solution to overcome the above-mentioned challenges (i.e., adverse side effects, such as neurotoxicity, cytokine storm, and hypogammaglobulinemia) associated with virally engineered CAR T cell therapy is utilizing mRNA transfection to induce CAR expression. mRNA allows for the transient expression of CAR, as it is translated without genomic integration (Riley R S et al., 2019, Nat Rev Drug Discov, 18:175-196). Furthermore, the customizable structure of in vitro transcribed (IVT) mRNA allows it to be engineered for maximized transfection and translation (Pardi N et al., 2018, Nat Rev Drug Discov, 17:261-279; Smits E et al., 2004, Leukemia, 18:1898-1902). mRNA-induced CAR T cell therapy has been validated in previous studies on a variety of cancers including ALL, melanoma, and Hodgkin's lymphoma, and it has been shown to reduce short term disease burden as effectively as stably expressing CAR T cells (Yang G et al., 2015, Cell, 344:1173-1178; Barrett D M et al., 2011, Hum Gene Ther, 22:1575-1586; Harrer D C et al., 2017, BMC Cancer, 17:551; Pardi N et al., 2015, J Control Release, 217:345-351; Svoboda J et al., 2018. Blood, 132:1022-1027; Rabinovich P M et al., 2006, Hum Gene Ther, 17: 1027-1035; Zhao Y et al., 2010, Cancer Res, 70:9053-9061; Singh N et al., 2014, Cancer Immunol Res, 2:1059-1070; Tasian S K et al., 2017, Blood, 129:2395-2407; Foster J B et al., 2018, Hum Gene Ther, 30:168-178; Barrett D M et al., 2014, Annu Rev Med, 65:333-347; Beatty G L et al., 2014, Cancer Immunol Res., 2:112-120; Yoon S H et al., 2009, Cancer Gene Ther, 16:489-497). Given this potential, mRNA CAR T cell therapy has numerous ongoing clinical trials for cancers including colorectal cancer and B cell lymphoma, among others (Foster J B et al., 2019, Mol Ther, 27:747-756). These previous investigations have confirmed that CAR expression typically persists for less than a week, which limits the ability of mRNA-induced therapy to offer long term therapeutic benefits without re-administration (Barrett D M et al., 2011, Hum Gene Ther, 22:1575-1586; Foster J B et al., 2018, Hum Gene Ther, 30:168-178; Beatty G L et al., 2014, Cancer Immunol Res., 2:112-120). However, it also has the potential to cause fewer on-target, off-tumor effects, and has been shown to offer lower toxicity (Barrett D M et al., 2011, Hum Gene Ther, 22:1575-1586; Zhao Y et al., 2010, Cancer Res, 70:9053-9061; Foster J B et al., 2018, Hum Gene Ther, 30:168-178; Barrett D M et al., 2014, Annu Rev Med, 65:333-347; Beatty G L et al., 2014, Cancer Immunol Res, 2:112-120).

Additionally, the amount of mRNA delivered to the T cells was shown to affect the level of CAR expression on T cells and allow for tunable toxicity, indicating that mRNA-based CAR expression may offer a means to mitigate the side effects, such as cytokine release, associated with CAR T cell therapy (Barrett D M et al., 2011, Hum Gene Ther, 22:1575-1586; Zhao Y et al., 2010, Cancer Res, 70:9053-9061). Previous studies have validated mRNA-induced CAR T cell therapy in a variety of cancers, and it has been shown to reduce short term disease burden as effectively as stably expressing CAR T cells (Yang G et al., 2015, Cell, 344:1173-1178; Barrett D M et al., 2011, Hum Gene Ther, 22:1575-1586; Harrer D C et al., 2017, BMC Cancer, 17:551; Pardi N et al., 2015, J Control Release, 217:345-351; Svoboda J et al., 2018, Blood, 132:1022-1027; Rabinovich P M et al., 2006, Hum Gene Ther, 17: 1027-1035; Zhao Y et al., 2010, Cancer Res, 70:9053-9061; Singh N et al., 2014, Cancer Immunol Res, 2:1059-1070; Tasian S K et al., 2017, Blood, 129:2395-2407; Foster J B et al., 2018, Hum Gene Ther, 30:168-178; Barrett D M et al., 2014, Annu Rev Med, 65:333-347; Beatty G L et al., 2014, Cancer Immunol Res., 2:112-120; Yoon S H et al., 2009, Cancer Gene Ther, 16:489-497). Given this potential, mRNA CAR T cell therapy has numerous ongoing clinical trials for cancers, including colorectal cancer and B cell lymphoma among others (Foster J B et al., 2019, Mol Ther, 27:747-756).

However, because naked mRNA degrades rapidly and cannot readily cross the cell membrane, it requires delivery methods to get functional uptake into T cells. Currently, electroporation (EP) is used clinically to effectively deliver mRNA to a variety of cells including T cells (Smits E et al., 2004, Leukemia, 18:1898-1902; Barrett D M et al., 2011. Hum Gene Ther, 22:1575-1586; DiTommaso T et al., 2018, PNAS, 115), but it has a number of disadvantages. The membrane disruption that occurs during EP risks the loss of cell content and cytotoxicity while failing to guarantee consistent membrane penetration across cells for even delivery. This can lead to low viability, and alter behavior of the surviving cell population (DiTommaso T et al., 2018, PNAS, 115; Dullaers M et al., 2004, Mol Ther, 10:768-779; Singh N et al., 2014, Cancer Immunol Res, 2:1059-1070). Thus, further investigation into the long term expression of transgenes and behavior in cells after electroporation is needed to understand the potential risks associated with this method of nucleic acid delivery (Lambricht L et al., 2016, Expert Opin Drug Deliv, 13: 295-310; Nickoloff J A et al., 1995, Animal Cell Electroporation and Electrofusion Protocols Methods in Molecular Biology, 273-280).

Thus, there is a need in the art for improved compositions and methods of delivering mRNA molecules encoding CAR to cells for treatment of various diseases or disorders. The present invention satisfies this unmet need.

### BRIEF SUMMARY OF THE INVENTION

In one aspect, the present invention relates, in part, to lipid nanoparticles (LNPs) comprising at least one mRNA molecule and at least one compound or salt thereof having the structure of Formula (I)

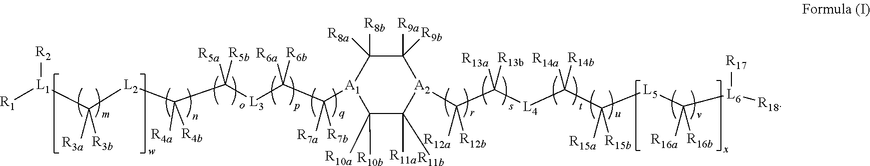

In some embodiments, A₁ and A₂ are independently C, C(H), N, S, or P. In some embodiments, each L₁, L₂, L₃, L₄, L₅, and L₆ is independently C, C(H)₂, C(H)(R₁₉), O, N(H), or N(R₁₉). In some embodiments, each R₁, R₂, R₃ₐ, R_{3b}, R₄ₐ, R_{4b}, R₅ₐ, R_{5b}, R₆ₐ, R_{6b}, R₇ₐ, R_{7b}, R₈ₐ, R_{8b}, R₉ₐ, R_{9b}, R₁₀ₐ, R_{10b}, R₁₁ₐ, R_{11b}, R₁₂ₐ, R_{12b}, R₁₃ₐ, R_{13b}, R₁₄ₐ, R_{14b}, R₁₅ₐ, R_{15b}, R₁₆, R₁₇, R₁₈, and R₁₉ is independently H, halogen, alkyl, substituted alkyl, cycloalkyl, substituted cycloalkyl, —Y(R₂₀)_{z′}(R₂₁)_{z″}-cycloalkyl, substituted —Y(R₂₀)_{z′}(R₂₁)_{z″}-cycloalkyl, heterocycloalkyl, substituted heterocycloalkyl, —Y(R₂₀)_{z′}(R₂₁)_{z″}-heterocycloalkyl, substituted-(R₂₀)_{z′}(R₂₁)_{z″}-heterocycloalkyl, alkenyl, substituted alkenyl, cycloalkenyl, substituted cycloalkenyl, —Y(R₂₀)_{z′}(R₂₁)_{z″}-cycloalkenyl, substituted —Y(R₂₀)_{z′}(R₂₁)_{z″}-cycloalkenyl, alkynyl, substituted alkynyl, cycloalkynyl, substituted cycloalkynyl, —Y(R₂₀)_{z′}(R₂₁)_{z″}-cycloalkynyl, substituted —Y(R₂₀)_{z′}(R₂₁)_{z″}-cycloalkynyl, aryl, substituted aryl, —Y(R₂₀)_{z′}(R₂₁)_{z″}-aryl, substituted —Y(R₂₀)_{z′}(R₂₁)_{z″}-aryl, heteroaryl, substituted heteroaryl, —Y(R₂₀)_{z′}(R₂₁)_{z″}-heteroaryl, substituted —Y(R₂₀)_{z′}(R₂₁)_{z″}-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R₂₀)_{z′}(R₂₁)_{z″}-ester, —Y(R₂₀)_{z′}(R₂₁)_{z″}, ═O, —NO₂, —CN, or sulfoxy.

In some embodiments, Y is C, N, O, S, or P. In some embodiments, each R₂₀ and R₂₁ is independently H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, or sulfoxy.

In some embodiments, each z′ and z″ is independently an integer represented by 0, 1, or 2. In some embodiments, each m, n, o, p, q, r, s, t, u, v, w, and x is independently an integer represented by 0, 1, 2, 3, 4, or 5.

In various embodiments, the mRNA molecule encodes a chimeric antigen receptor (CAR).

In some embodiments, the compound having the structure of Formula (I) is a compound having the structure of:

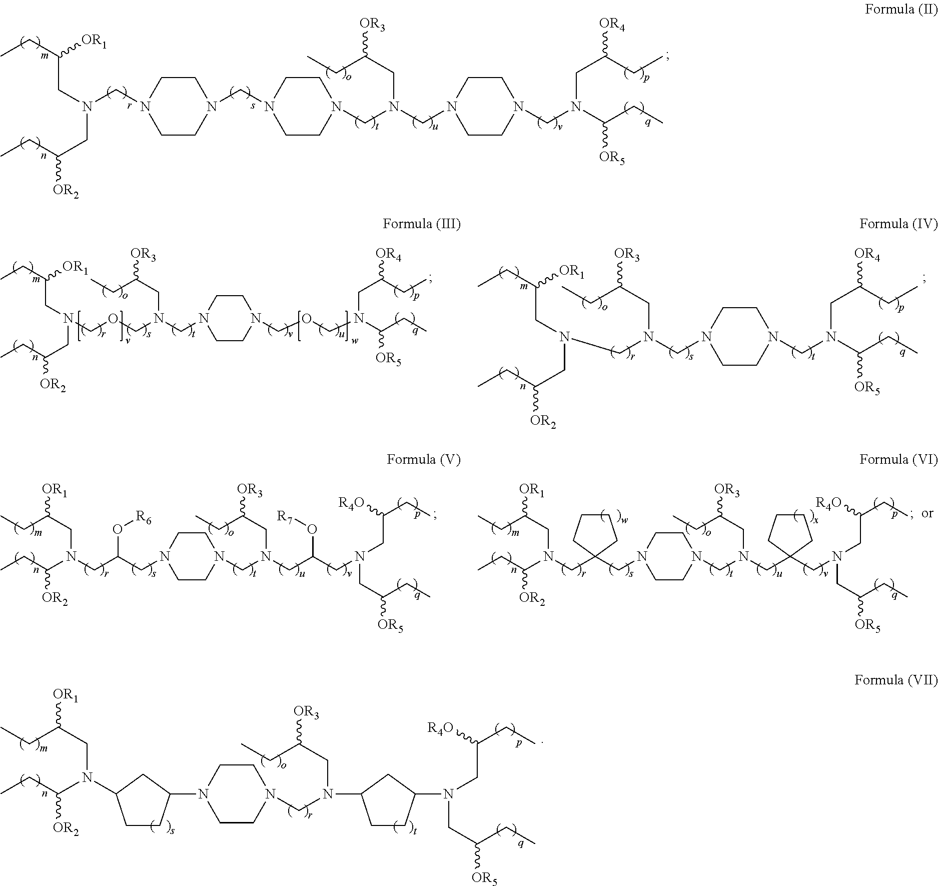

In some embodiments, each R₁, R₂, R₃, R₄, and R₅ is independently H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, or ester.

In some embodiments, each m, n, o, p, and q is independently an integer from 0 to 25. In some embodiments, each r, s, t, u, v, w, and x is independently an integer represented by 0, 1, 2; 3, 4, or 5.

In some embodiments, the compound having the structure of Formula (I) is a compound having the structure of:

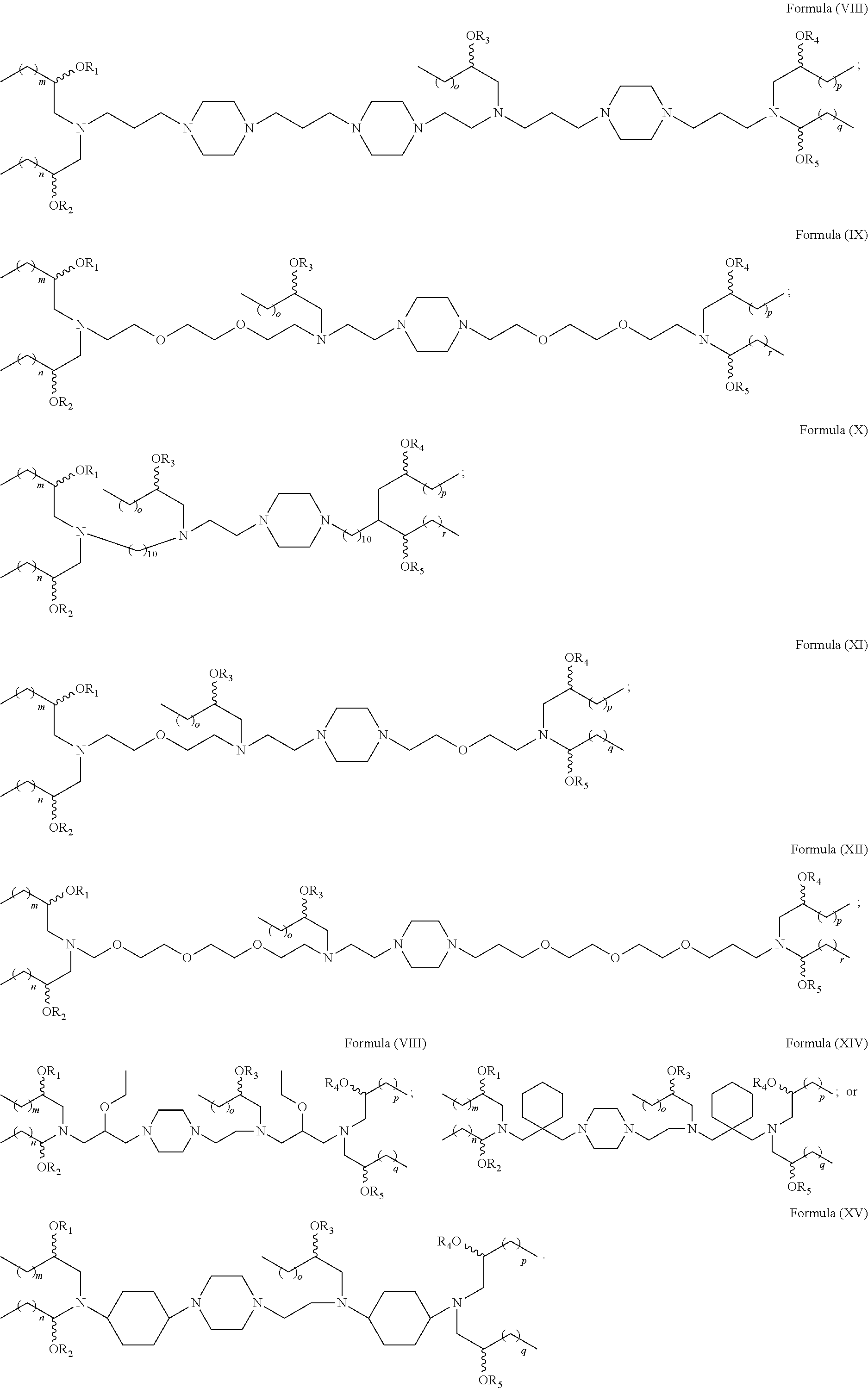

In some embodiments, the compound having the structure of Formula (I) is an ionizable lipid. In some embodiments, the mRNA molecule is encapsulated within the compound having the structure of Formula (I).

In some embodiments, the LNP comprises the compound or salt thereof having the structure of Formula (I) in a concentration range of about 1 mol % to about 100 mol %. In some embodiments, the LNP comprises the compound or salt thereof having the structure of Formula (I) in a concentration range of about 10 mol % to about 50 mol %.

In some embodiments, the LNP further comprises at least one helper lipid. In some embodiments, the LNP comprises at least one helper lipid in a concentration range of about 0.01 mol % to about 99.9 mol %. In some embodiments, the LNP comprises at least one helper lipid in a concentration range of about 0.5 mol % to about 50 mol %.

In some embodiments, the helper lipid is phospholipid, cholesterol lipid, polymer, or any combination thereof.

In some embodiments, the phospholipid is dioleoyl-phosphatidylethanolamine (DOPE) or a derivative thereof, distearoylphosphatidylcholine (DSPC) or a derivative thereof, distearoyl-phosphatidylethanolamine (DSPE) or a derivative thereof, stearoyloleoylphosphatidylcholine (SOPC) or a derivative thereof, 1-stearioyl-2-oleoyl-phosphatidyethanol amine (SOPE) or a derivative thereof, N-(2,3-dioleoyloxy)propyl)-N,N,N-trimethylammonium chloride (DOTAP) or a derivative thereof, or any combination thereof. In some embodiments, the LNP comprises a phospholipid in a concentration range of about 15 mol % to about 50 mol %.

In some embodiments, the cholesterol lipid is cholesterol or a derivative thereof. In some embodiments, the LNP comprises a cholesterol lipid in a concentration range of about 20 mol % to about 50 mol %.

In some embodiments, the polymer is polyethylene glycol (PEG) or a derivative thereof. In some embodiments, the LNP comprises a polymer in a concentration range of about 0.5 mol % to about 10 mol %.

In some embodiments, the LNP further comprises at least one nucleic acid molecule, adjuvant, therapeutic agent, or any combination thereof. In one embodiment, the nucleic acid molecule is a therapeutic agent.

In some embodiments, the nucleic acid molecule is a DNA molecule or an RNA molecule. In some embodiments, the nucleic acid molecule is cDNA, mRNA, miRNA, siRNA, sgRNA, modified RNA, antagomir, antisense molecule, guide RNA molecule, CRISPR guide RNA molecule, peptide, therapeutic peptide, targeted nucleic acid, or any combination thereof.

In some embodiments, the mRNA encodes one or more antigens. In some embodiments, the antigen comprises at least one viral antigen, a bacterial antigen, a fungal antigen, a parasitic antigen, an influenza antigen, a tumor-associated antigen, a tumor-specific antigen, or any combination thereof.

In some embodiments, the nucleic acid molecule comprises a promoter or regulatory sequence.

In some embodiments, the mRNA molecule encoding a CAR, nucleic acid molecule, adjuvant, therapeutic agent, or any combination thereof is encapsulated within the compound having the structure of Formula (I).

In another aspect, the present invention also relates, in part, to a composition comprising at least one LNP described herein. In one embodiment, the composition is a vaccine.

In another aspect, the present invention also relates, in part, to a method of delivering at least one mRNA molecule encoding CAR to a subject in need thereof. In various embodiments, the method comprises administering a therapeutically effectively amount of one or more LNPs or compositions thereof described herein to the subject. In some embodiments, the LNP or the composition thereof delivers the mRNA molecule encoding CAR to a target.

In some embodiments, the target is an immune cell, T cell, resident T cells, B cell, natural killer (NK) cell, cancerous cell, cell associated with a disease or disorder, tissue associated with a disease or disorder, brain tissue, central nervous system tissue, pulmonary tissue, apical surface tissue, epithelial cell, endothelial cell, liver tissue, intestine tissue, colon tissue, small intestine tissue, large intestine tissue, feces, bone marrow, macrophages, spleen tissue, muscles tissue, joint tissue, tumor cells, diseased tissues, lymph node tissue, lymphatic circulation, or any combination thereof.

In some embodiments, the method comprises a single administration of the LNP or the composition thereof. In some embodiments, the method comprises multiple administrations of the LNP or the composition thereof.

In some embodiments, the LNP or the composition thereof is administered by a delivery route selected from the group consisting of intradermal, subcutaneous, intramuscular, intraventricular, intrathecal, oral delivery, intravenous, intratracheal, intraperitoneal, in utero delivery, or any combination thereof.

In some embodiments, the LNP or the composition thereof further comprises at least one nucleic acid molecule, adjuvant, therapeutic agent, or any combination thereof.

In some embodiments, the method treats or prevents at least one selected from the group consisting of a viral infection, a bacterial infections, a fungal infection, a parasitic infection, influenza infection, cancer, arthritis, heart disease, cardiovascular disease, neurological disorder or disease, genetic disease, autoimmune disease, fetal disease, genetic disease affecting fetal development, or any combination thereof.

In one embodiment, the LNP composition is a vaccine.

In another aspect, the present invention also relates, in part, to a method of delivering at least one mRNA molecule encoding CAR and at least one nucleic acid molecule, adjuvant, therapeutic agent, or any combination thereof to a subject in need thereof. In various embodiments, the method comprises administering a therapeutically effectively amount of one or more LNPs or compositions thereof described herein to the subject. In some embodiments, the LNP or the composition thereof delivers the mRNA molecule and the nucleic acid molecule, adjuvant, therapeutic agent, or any combination thereof to a target. Thus, in various embodiments, the method is a gene delivery method.

In another aspect, the present invention also relates, in part, to a method of preventing or treating a disease or disorder in a subject in need thereof. In various embodiments, the method comprises administering a therapeutically effectively amount of one or more LNPs or compositions thereof described herein to the subject.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1C, depicts schematic representation of LNP formulation and CAR mRNA loaded LNPs. FIG. 1A depicts schematic of the components used to generate LNPs via microfluidic mixing and the expected structure of the resulting LNPs. FIG. 1B depicts the size (z-average) distribution of a representative sample of C14-4 (also referred to as C14-494) LNPs revealing a diameter of approximately 70 nm using dynamic light scattering. Error bars represent standard deviation across three samples. FIG. 1C depicts schematic of CAR mRNA loaded LNPs inducing CAR expressing in T cells and resulting in tumor cell targeting to eliminate cancerous cells.

FIG. 2, comprising FIG. 2A through FIG. 2C, depicts representative epoxide-terminated alkyl chains and representative polyamine cores used to create the library of lipids screened in this investigation. The lipids were made via Michael addition chemistry. The invention described here is C14-4 (also referred to as C14-494) as named by this diagram. FIG. 2A depicts representative structures of the lipid tails used to generate the ionizable lipid library. FIG. 2B depicts representative structures of the amine cores used to generate the ionizable lipid library. FIG. 2C depicts a schematic representation of the Michael addition reaction chemistry used to synthesize the ionizable lipids by reacting an excess of lipid tails with the amine cores.

FIG. 3, comprising FIG. 3A through FIG. 3E, depicts representative luciferase expression under a variety of conditions. Results normalized to untreated cells with background subtracted. n=3 for FIG. 3B, FIG. 3D, and FIG. 3E. FIG. 3A depicts representative luciferase expression of Jurkat cells after treatment with the LNP library and lipofectamine for 48 hr at a dose of 30 ng/60,000 cells revealing top performing LNPs. Results were normalized to untreated cells and background was subtracted. *=p<0.05 in paired student T test to lipofectamine, n=4. FIG. 3B depicts representative luciferase expression of Jurkat cells treated with the top five performing LNP formulations to determine top LNP formulation. Results were normalized to untreated cells and background was subtracted. *=p<0.05 in tukey's multiple comparison test between C14-4 (also referred to as C14-494) and each other formulation. FIG. 3C depicts a table reporting the representative diameters (z-average), polydispersity index, and mRNA concentration (i standard deviation) of the top five LNP formulations. FIG. 3D depicts representative luciferase expression over time in Jurkat cells treated with 30 ng/60,000 cells of C14-4 (also referred to as C14-494) for 24 hr confirmed transient expression of the protein. Results normalized to expression at 24 hr with background subtracted. FIG. 3E depicts representative viability of Jurkat cells treated with 30 ng mRNA/60,000 cells for 48 hr using lipofectamine or C14-4 (also referred to as C14-494) showing minimal toxicity associated with the C14-4 (also referred to as C14-494) LNP.

FIG. 4, comprising FIG. 4A through FIG. 4C, depicts representative luciferase expression under a variety of conditions. For FIG. 4A and FIG. 4C, luciferase expression normalized to lowest treatment (75 ng/60,000 cells) and viability normalized to no treatment with background subtracted. n=3. FIG. 4A depicts representative luciferase expression and viability of primary T cells treated with crude C14-4 (also referred to as C14-494) LNPs for 24 hr. FIG. 4B depicts representative results of TNS assay to determine LNP pKa for the crude and pure C14-4 (also referred to as C14-494) LNPs encapsulating luciferase mRNA. pKa was calculated as the pH corresponding to half of the maximum TNS fluorescence value. FIG. 4C depicts representative luciferase expression and viability of primary T cells treated with either crude or purified C14-4 (also referred to as C14-494) showing increased luciferase expression with no increase in toxicity. *=p<0.05 in paired student T test.

FIG. 5, comprising FIG. 5A through FIG. 5D, depicts representative results for CAR T cells. FIG. 5A depicts representative results demonstrating that surface expression of CAR on primary T cells assessed using flow cytometry revealed increased expression evaluated as mean fluorescent intensity (MFI) in pure C14-4 (also referred to as C14-494) and EP treatment groups compared to crude C14-4 (also referred to as C14-494). FIG. 5B depicts a comparison of representative MFI values normalized to the untreated control. FIG. 5C depicts representative viability of primary T cells treated with each group normalized to treatment with background subtracted, n=3. FIG. 5D depicts representative results of Nalm6 and CAR T cell co-plating at different effector-to-target ratios for 48 hr. n=3 wells.

FIG. 6 depicts representative named structures of the amine cores used to generate the ionizable lipid library.

FIG. 7 depicts representative diameter (z-average), PDI, and mRNA concentration of each LNP formulation showing a narrow range in LNP size, monodispersity, and similar mRNA loading across LNP formulations.

FIG. 8 depicts representative comparison of characteristics of crude and pure C14-4 (also referred to as C14-494) LNPs encapsulating luciferase mRNA. Averages n=3+ with t standard deviation.

FIG. 9, comprising FIG. 9A and FIG. 9B, depicts representative Library A formulations with the different mole ratio % s screened and data showing that the ionizable lipids (e.g., C14-494) were all still ionizable when incorporated into an LNP. In the present study, the “S2” formulation was set as the standard C14-494 formulation of excipients. FIG. 9A depicts representative fabrication parameters for representative Library A formulations, expressed as molar ratios. FIG. 9B depicts representative pKa ratios for Library A formulations. The pKa of Library A, using a TNS assay, showed that all were still ionizable

FIG. 10 depicts representative fabrication parameters for representative Library A formulations with different mole ratio % s. The selection of these representative formulations was selected based on the outcomes from screening Library A.

FIG. 11 depicts representative normalized delivery effectiveness of both libraries. Value greater than one (dashed line) indicate an increase in delivery effectiveness over the positive control.

FIG. 12 depicts representative normalized cellular viability of Library A formulations. The dashed line marks 100% viability.

FIG. 13 depicts representative normalized cellular viability of Library B formulations. The dashed line marks 100% viability.

FIG. 14, comprising FIG. 14A and FIG. 14B, depicts representative mRNA delivery and viability excipient composition: libraries in vitro. Jurkats were treated for 24 hr with 30 ng/60,0000 cells. Library A is shown in orange for comparison; Library B is shown in blue. FIG. 14A depicts representative mRNA delivery excipient composition: libraries in vitro. FIG. 14B depicts representative viability delivery excipient composition: libraries in vitro.

FIG. 15 depicts representative results demonstrating relative luciferase activity for B10 and lipofectamine.

FIG. 16, comprising FIG. 16A and FIG. 16B, depicts representative luminescence and viability results for various representative formulations at different concentrations/doses. Jurkats were treated for 24 hr (the “S2” formulation was set as the standard C14-494 formulation of excipients) with luciferase-encoding mRNA. *normalized to 0 ng for luminescence and toxicology—the values graphed in FIG. 16A and FIG. 16B for all treatment groups are values that have been normalized to untreated groups. More specifically, the luminescent and toxicity readings for each treatment group were measures of luminescence. The raw value (luminescence) for each treatment was divided by the raw value (luminescence) measured in the group of cells that received no treatment. Thus, the graphed values represent the delivery or toxicity as compared to untreated cells. This allowed for background luminescence-which varied between experiments—to be removed as a factor. Furthermore, this experiment was completed at three separate times with three separate Jurkat cell populations/passages (three biological replicates), and in each experiment the cells were plated in triplicate wells (three technical replicates). Thus, ensuring that the results were repeatable (biological replicates) and reliable (technical replicates). FIG. 16A depicts representative luminescence results for various representative formulations at different concentrations.

FIG. 16B depicts representative viability results for various representative formulations at different concentrations.

FIG. 17, comprising FIG. 17A through FIG. 17C, depicts representative excipient composition: ex vivo for patient A, patient B, and patient C.

FIG. 17A depicts representative excipient composition: ex vivo for patient A. FIG. 17B depicts representative excipient composition: ex vivo for patient B. FIG. 17C depicts representative excipient composition: ex vivo for patient C.

### DETAILED DESCRIPTION

The present invention relates to lipid nanoparticles (LNPs) or compositions thereof for delivery of mRNA encoding chimeric antigen receptor (CAR) into various targets (e.g., cells). In various embodiments, the invention relates to methods for delivery of mRNA encoding CAR as well as various nucleic acid molecules, adjuvants, and/or therapeutic agents into various targets (e.g., cells) using at least one LNP or a composition thereof. In some embodiments, the LNP or the composition thereof comprises at least one ionizable lipid and at least one helper lipid. In certain embodiments, the invention provides the LNPs or the compositions thereof comprising at least one ionizable lipid and at least one mRNA molecule encoding CAR for preventing or treating various diseases or disorders in a subject in need thereof.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably +5%, even more preferably +1%, and still more preferably ±0.1% from the specified value, as such variations are appropriate to perform the disclosed methods.

“Alkyl” refers to a straight or branched hydrocarbon chain radical consisting solely of carbon and hydrogen atoms, which is saturated or unsaturated (i.e., contains one or more double and/or triple bonds), having from one to twenty-four carbon atoms (C₁-C₂₄ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond, e.g., methyl, ethyl, n propyl, 1-methylethyl (iso propyl), n butyl, n pentyl, 1,1 dimethylethyl (t butyl), 3 methylhexyl, 2 methylhexyl, ethenyl, prop 1 enyl, but-1-enyl, pent-1-enyl, penta-1,4-dienyl, ethynyl, propynyl, butynyl, pentynyl, hexynyl, and the like. Unless specifically stated otherwise, an alkyl group is optionally substituted. The term “alkyl,” by itself or as part of another substituent means, unless otherwise stated, a straight or branched chain hydrocarbon having the number of carbon atoms designated (i.e., C₁₋₆ means one to six carbon atoms) and includes straight, branched chain, or cyclic substituent groups.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C═N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double (alkenylene) and/or triple bonds (alkynylene)), and having, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the 15 radical group can be through one carbon or any two carbons within the chain. Unless stated otherwise specifically in the specification, an alkylene chain may be optionally substituted.

“Cycloalkyl” or “carbocyclic ring” refers to a stable non aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7 dimethyl bicyclo[2.2.1]heptanyl, and the like. Unless specifically stated otherwise, a cycloalkyl group is optionally substituted.

“Cycloalkylene” is a divalent cycloalkyl group. Unless otherwise stated specifically in the specification, a cycloalkylene group may be optionally substituted.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, Si, P, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless specifically stated otherwise, a heterocyclyl group may be optionally substituted.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “heteroaryl” or “heteroaromatic” refers to aryl groups which contain at least one heteroatom selected from N, O, Si, P. and S; wherein the nitrogen and sulfur atoms may be optionally oxidized, and the nitrogen atom(s) may be optionally quaternized. Heteroaryl groups may be substituted or unsubstituted. A heteroaryl group may be attached to the remainder of the molecule through a heteroatom. A polycyclic heteroaryl may include one or more rings that are partially saturated. Examples include tetrahydroquinoline, 2,3-dihydrobenzofuryl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), 25 isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the terms “alkoxy,” “alkylamino” and “alkylthio” are used in their conventional sense, and refer to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

The term “substituted” used herein means any of the above groups (e.g., alkyl, cycloalkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (—OR^{a}, where R^{a} is C₁-C₁₂ alkyl or cycloalkyl); carboxyl groups (—OC(═O)R^{a} or —C(═O)OR^{a}, where R^{a} is H, C₁-C₁₂ alkyl or cycloalkyl); amine groups (—NR^{a}R^{b}, where R^{a} and R^{b} are each independently H, C₁-C₁₂ alkyl or cycloalkyl); C₁-C₁₂ alkyl groups; and cycloalkyl groups. In some embodiments the substituent is a C₁-C₁₂ alkyl group. In other embodiments, the substituent is a cycloalkyl group. In other embodiments, the substituent is a halo group, such as fluoro. In other embodiments, the substituent is a oxo group. In other embodiments, the substituent is a hydroxyl group. In other embodiments, the substituent is an alkoxy group. In other embodiments, the substituent is a carboxyl group. In other embodiments, the substituent is an amine group.

As used herein, the term “nanoparticle” refers to particles having a particle size on the nanometer scale, less than 1 micrometer. For example, the nanoparticle may have a particle size up to about 50 nm. In another example, the nanoparticle may have a particle size up to about 10 nm. In another example, the nanoparticle may have a particle size up to about 6 nm. As used herein, “nanoparticle” refers to a number of nanoparticles, including, but not limited to, nanoclusters, nanovesicles, micelles, lamaellae shaped particles, polymersomes, dendrimers, and other nano-size particles of various other small fabrications that are known to those in the art. The shapes and compositions of nanoparticles may be guided during condensation of atoms by selectively favoring growth of particular crystal facets to produce spheres, rods, wires, discs, cages, core-shell structures and many other shapes. The definitions and understandings of the entities falling within the scope of nanocapsule are known to those of skill in the art, and such definitions are incorporated herein by reference and for the purposes of understanding the general nature of the subject matter of the present application.

As used herein, “nucleic acid” is meant to include any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). The term “nucleic acid” typically refers to large polynucleotides.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment, which has been separated from sequences which flank it in a naturally occurring state, i.e., a DNA fragment, which has been removed from the sequences which are normally adjacent to the fragment, i.e., the sequences adjacent to the fragment in a genome in which it naturally occurs. The term also applies to nucleic acids which have been substantially purified from other components, which naturally accompany the nucleic acid, i.e., RNA or DNA or proteins, which naturally accompany it in the cell. The term therefore includes, for example, a recombinant DNA or RNA, which is incorporated into a vector, into an autonomously replicating plasmid or virus, or into the genomic DNA or RNA of a prokaryote or eukaryote, or which exists as a separate molecule (i.e., as a cDNA or a genomic or cDNA fragment produced by PCR or restriction enzyme digestion) independent of other sequences. It also includes a recombinant DNA or RNA, which is part of a hybrid gene encoding additional polypeptide sequence.

A “coding region” of an mRNA molecule also consists of the nucleotide residues of the mRNA molecule, which are matched with an anti-codon region of a transfer RNA molecule during translation of the mRNA molecule, or which encode a stop codon. The coding region may thus include nucleotide residues comprising codons for amino acid residues, which are not present in the mature protein encoded by the mRNA molecule (e.g., amino acid residues in a protein export signal sequence).

The term “DNA” as used herein is defined as deoxyribonucleic acid.

The term “RNA” as used herein is defined as ribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Immunogen” refers to any substance introduced into the body in order to generate an immune response. That substance can a physical molecule, such as a protein, or can be encoded by a vector, such as DNA, mRNA, or a virus.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translation by translational machinery in a cell.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the invention is a “nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “recombinant polypeptide” as used herein is defined as a polypeptide produced by using recombinant DNA or RNA methods.

The term “recombinant DNA” as used herein is defined as DNA produced by joining pieces of DNA from different sources.

The term “recombinant RNA” as used herein is defined as RNA produced by joining pieces of RNA from different sources.

As used herein, the term “identical” refers to two or more sequences or subsequences which are the same.

In addition, the term “substantially identical,” as used herein, refers to two or more sequences which have a percentage of sequential units which are the same when compared and aligned for maximum correspondence over a comparison window, or designated region as measured using a comparison algorithm or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring, such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85% identical to the reference sequence.

As used herein, “fragment” is defined as at least a portion of the variable region of the immunoglobulin molecule which binds to its target, i.e. the antigen binding region. Some of the constant region of the immunoglobulin may be included.

As used herein, the term “linkage” refers to bonds or chemical moiety formed from a chemical reaction between the functional group of a linker and another molecule. Such bonds may include, but are not limited to, covalent linkages and non-covalent bonds, while such chemical moieties may include, but are not limited to, esters, carbonates, imines phosphate esters, hydrazones, acetals, orthoesters, peptide linkages, and oligonucleotide linkages. Hydrolytically stable linkages means that the linkages are substantially stable in water and do not react with water at useful pH values, including but not limited to, under physiological conditions for an extended period of time, perhaps even indefinitely. Hydrolytically unstable or degradable linkages means that the linkages are degradable in water or in aqueous solutions, including for example, blood. Enzymatically unstable or degradable linkages means that the linkage can be degraded by one or more enzymes. By way of example only, PEG and related polymers may include degradable linkages in the polymer backbone or in the linker group between the polymer backbone and one or more of the terminal functional groups of the polymer molecule. Such degradable linkages include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

The term “gene,” as used herein, refers to a nucleic acid molecule that encodes a protein or functional RNA (for example, a tRNA). A gene can include regions that do not encode the final protein or RNA product, such as 5′ or 3′ untranslated regions, introns, ribosome binding sites, promoter or enhancer regions, or other associated and/or regulatory sequence regions.

The terms “gene expression” and “expression” are used interchangeably herein to refer to the process by which inheritable information from a gene, such as a DNA sequence, is made into a functional gene product, such as protein or RNA.

As used herein, the terms “promoter” or “regulatory sequence” mean a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “Chimeric Antigen Receptor” or alternatively a “CAR” refers to a recombinant polypeptide construct comprising at least an extracellular antigen binding domain, a transmembrane domain, and a cytoplasmic signaling domain comprising a functional signaling domain derived from a stimulatory molecule as defined below. In one aspect, the stimulatory molecule is the zeta chain associated with the T cell receptor complex. In one aspect, the cytoplasmic signaling domain further comprises one or more functional signaling domains derived from at least one costimulatory molecule as defined below. In one aspect, the costimulatory molecule is chosen from 4 1BB (i.e., CD137), CD3, and/or CD28. In one aspect, the CAR comprises a chimeric fusion protein comprising an extracellular antigen recognition domain, a transmembrane domain, and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the scFv domain during cellular processing and localization of the CAR to the cellular membrane.

The portion of the CAR composition comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In one embodiment, the CAR comprises an antibody fragment that comprises a scFv.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “adjuvant” as used herein is defined as any molecule to enhance an antigen-specific adaptive immune response.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

An “effective amount” as used herein, means an amount which provides a therapeutic or prophylactic benefit.

The term “therapeutic” as used herein means a treatment and/or prophylaxis. A therapeutic effect is obtained by suppression, diminution, remission, or eradication of at least one sign or symptom of a disease or disorder state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

The terms “patient,” “subject,” “individual,” and the like are used interchangeably herein, and refer to any animal, or cells thereof or any multicellular organism, or cells thereof, whether in vitro or in situ, amenable to the methods described herein. In certain non-limiting embodiments, the patient, subject or individual is a human. In certain non-limiting embodiments, the patient, subject or individual is a fetus. In certain non-limiting embodiments, the patient, subject or individual is an embryo.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates, in part, to the discovery that novel lipid nanoparticle (LNP) or a composition thereof delivered mRNA molecules encoding CAR to T cells with enhanced efficiency and low toxicity. Thus, in some aspects, the present invention also relates to methods for delivery of mRNA encoding CAR as well as various nucleic acid molecules, adjuvants, and/or therapeutic agents into various targets (e.g., cells, tissues, etc.) using at least one LNP or a composition thereof. In some embodiments, the LNP or the composition thereof comprises at least one ionizable lipid and at least one helper lipid. In certain embodiments, the invention provides methods of preventing or treating diseases or disorders in a subject in need thereof using at least one LNP or a composition thereof comprising at least one mRNA molecule and at least one LNP.

### Lipid Nanoparticles (LNPs) and Compositions Thereof

The present invention relates, in part, to novel lipid nanoparticles (LNPs) comprising at least one mRNA molecule and at least one lipid. In one aspect, the present invention relates, in part, to compositions comprising at least one LNP of the present invention. Thus, in various aspects, the present invention relates, in part, to novel LNP compositions comprising at least one mRNA molecule and at least one lipid.

In various embodiments, the lipid is an ionizable lipid. In various embodiments, the lipid is a compound or salt thereof having the structure of Formula (I)

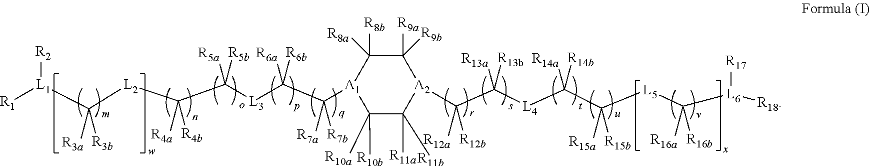

In some embodiments, A₁ is C, C(H), N, S, or P. In some embodiments, A₂ is C, C(H), N, S, or P.

In some embodiments, L₁ is C, C(H)₂, C(H)(R₁₉), O, N(H), or N(R₁₉). In some embodiments, L₂ is C, C(H)₂, C(H)(R₁₉), O, N(H), or N(R₁₉). In some embodiments, L₃ is C, C(H)₂, C(H)(R₁₉), O, N(H), or N(R₁₉). In some embodiments, L₄ is C, C(H)₂, C(H)(R₁₉), O, N(H), or N(R₁₉). In some embodiments, L₅ is C, C(H)₂, C(H)(R₁₉), O, N(H), or N(R₁₉). In some embodiments, L₆ is C, C(H)₂, C(H)(R₁₉), O, N(H), or N(R₁₉).

In some embodiments, R₁, R₂, R₃ₐ, R_{3b}, R₄ₐ, R_{4b}, R₅ₐ, R_{5b}, R₆ₐ, R_{6b}, R₇ₐ, R_{7b}, R₈ₐ, R_{8b}, R₉ₐ, R_{9b}, R₁₀ₐ, R_{10b}, R₁₁ₐ, R_{11b}, R₁₂ₐ, R_{12b}, R₁₃ₐ, R_{13b}, R₁₄ₐ, R_{14b}, R₁₅ₐ, R_{15b}, R₁₆, R₁₇, R₁₈, or R₁₈ is H, halogen, alkyl, substituted alkyl, cycloalkyl, substituted cycloalkyl, —Y(R₂₀)_{z′}(R₂₁)_{z″}-cycloalkyl, substituted —Y(R₂₀)_{z′}(R₂₁)_{z″}-cycloalkyl, heterocycloalkyl, substituted heterocycloalkyl, —Y(R₂₀)_{z′}(R₂₁)_{z″}-heterocycloalkyl, substituted-(R₂₀)_{z′}(R₂₁)_{z″}-heterocycloalkyl, alkenyl, substituted alkenyl, cycloalkenyl, substituted cycloalkenyl, —Y(R₂₀)_{z′}(R₂₁)_{z″}-cycloalkenyl, substituted —Y(R₂₀)_{z′}(R₂₁)_{z″}-cycloalkenyl, alkynyl, substituted alkynyl, cycloalkynyl, substituted cycloalkynyl, —Y(R₂₀)_{z′}(R₂₁)_{z″}-cycloalkynyl, substituted —Y(R₂₀)_{z′}(R₂₁)_{z″}-cycloalkynyl, aryl, substituted aryl, —Y(R₂₀)_{z′}(R₂₁)_{z″}-aryl, substituted —Y(R₂₀)_{z′}(R₂₁)_{z″}-aryl, heteroaryl, substituted heteroaryl, —Y(R₂₀)_{z′}(R₂₁)_{z″}-heteroaryl, substituted —Y(R₂₀)_{z′}(R₂₁)_{z″}-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R₂₀)_{z′}(R₂₁)_{z″}-ester, —Y(R₂₀)_{z′}(R₂₁)_{z″}, ═O, —NO₂, —CN, or sulfoxy.

In various embodiments, Y is C, N, O, S, or P.

In some embodiments, R₂₀ or R₂₁ is H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, or sulfoxy.

In some embodiments, z′ is an integer represented by 0, 1, 2, or 3. In some embodiments, z″ is an integer represented by 0, 1, 2, or 3.

In some embodiments, m, n, o, p, q, r, s, t, u, v, w, or x in an integer from 0 to 25. In various embodiments, m, n, o, p, q, r, s, t, u, v, w, or x in an integer represented by 0, 1, 2; 3, 4, or 5.

In some embodiments, the compound having the structure of Formula (I) is a compound having the structure of:

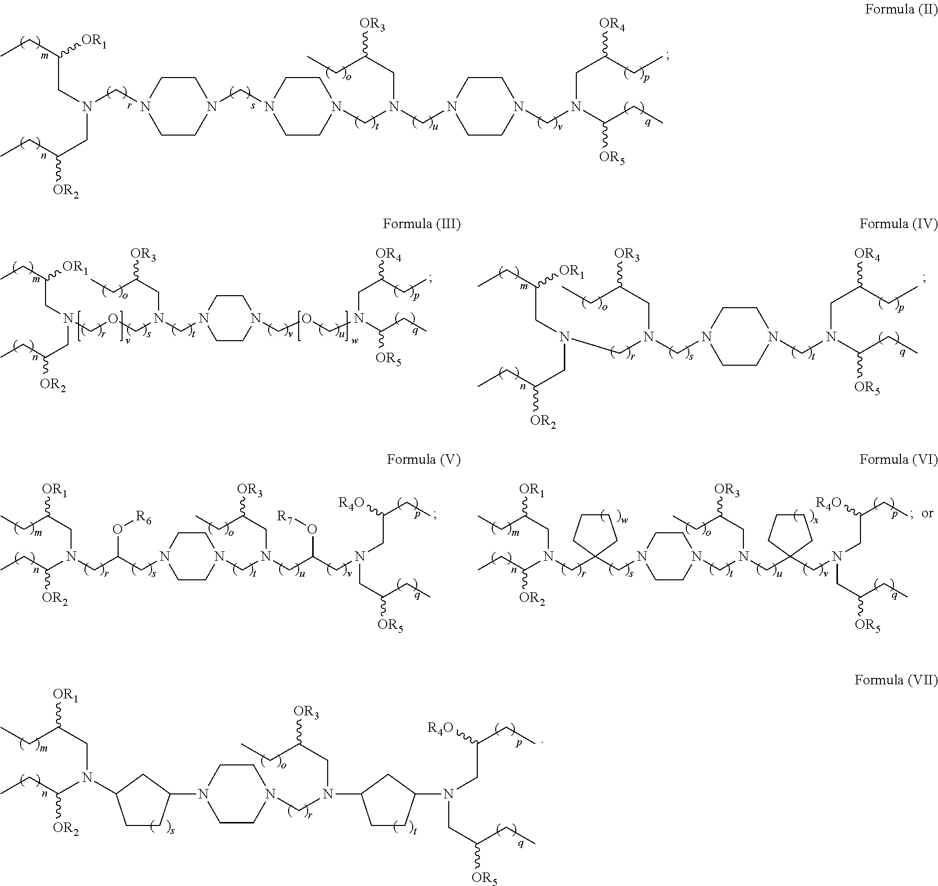

Thus, in various embodiments, R₁, R₂, R₃, R₄, or R₅ is H. halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, or ester.

In some embodiments, m, n, o, p, or q is an integer from 0 to 25. In some embodiments, r, s, t, u, v, w, or x is an integer represented by 0, 1, 2; 3, 4, and 5.

In some embodiments, the compound having the structure of Formula (I) is a compound having the structure of.

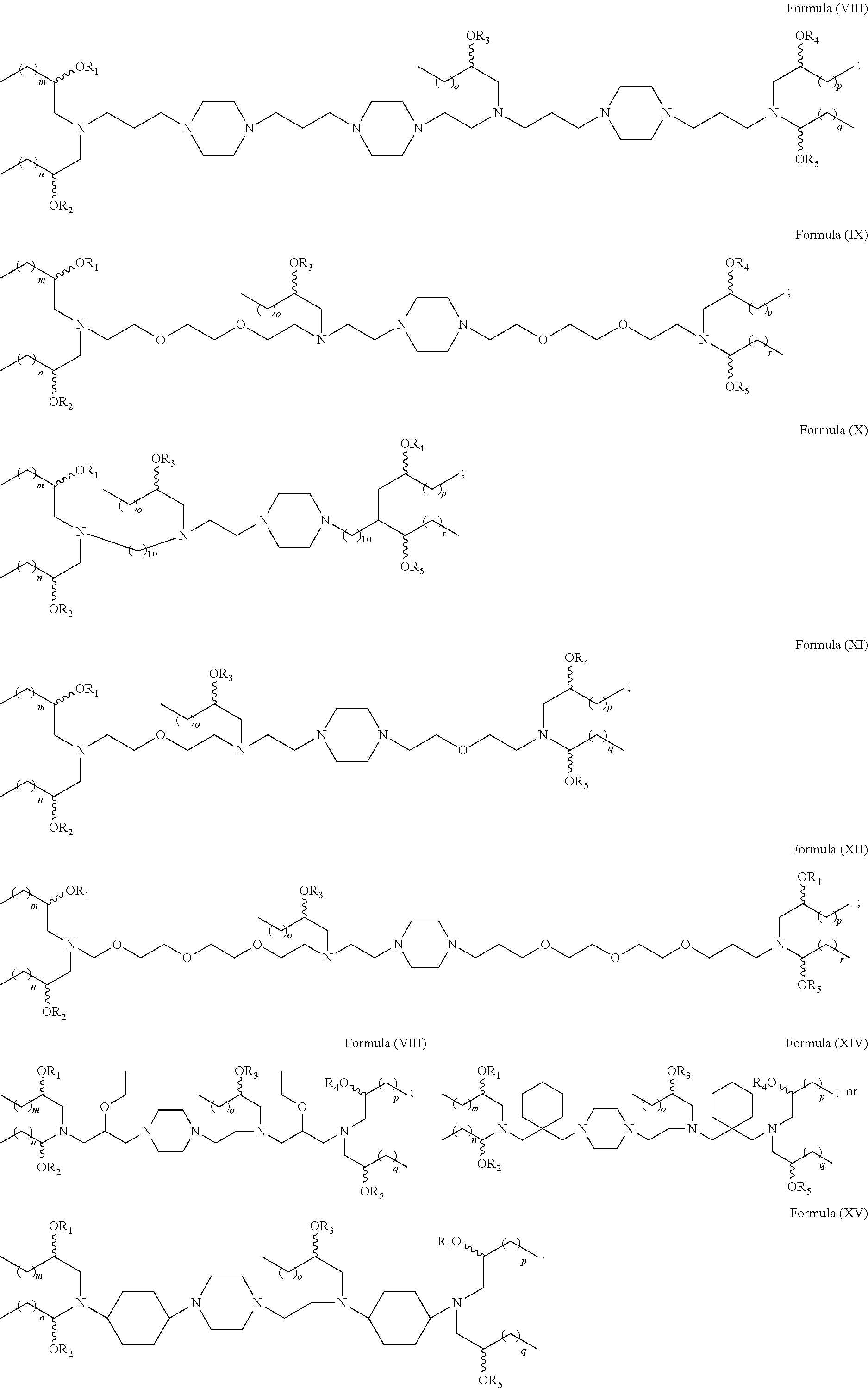

In various embodiments, the mRNA molecule encodes a chimeric antigen receptor (CAR). In some embodiments, the mRNA molecule is encapsulated within the compound having the structure of Formula (I). In some embodiments, the mRNA molecule is encapsulated within the compound having the structure of Formula (I)-(XV).

In various embodiments, the LNP or composition thereof comprises one or more lipids in a concentration range of about 0.1 mol % to about 100 mol %. In some embodiments, the LNP or composition thereof comprises one or more lipids in a concentration range of about 1 mol % to about 100 mol %. In some embodiments, the LNP or composition thereof comprises one or more lipids in a concentration range of about 10 mol % to about 70 mol %. In some embodiments, the LNP or composition thereof comprises one or more lipids in a concentration range of about 10 mol % to about 50 mol %. In some embodiments, the LNP or composition thereof comprises one or more lipids in a concentration range of about 15 mol % to about 45 mol %. In some embodiments, the LNP or composition thereof comprises one or more lipids in a concentration range of about 35 mol % to about 40 mol %.

For example, in some embodiments, the LNP or composition thereof comprises one or more compounds having the structure of Formula (I)-(XV) in a concentration of about 1 mol %. In some embodiments, the LNP or composition thereof comprises one or more compounds having the structure of Formula (I)-(XV) in a concentration of about 2 mol %. In some embodiments, the LNP or composition thereof comprises one or more compounds having the structure of Formula (I)-(XV) in a concentration of about 5 mol %. In some embodiments, the LNP or composition thereof comprises one or more compounds having the structure of Formula (I)-(XV) in a concentration of about 5.5 mol %. In some embodiments, the LNP or composition thereof comprises one or more compounds having the structure of Formula (I)-(XV) in a concentration of about 10 mol %. In some embodiments, the LNP or composition thereof comprises one or more compounds having the structure of Formula (I)-(XV) in a concentration of about 12 mol %. In some embodiments, the LNP or composition thereof comprises one or more compounds having the structure of Formula (I)-(XV) in a concentration of about 15 mol %. In some embodiments, the LNP or composition thereof comprises one or more compounds having the structure of Formula (I)-(XV) in a concentration of about 20 mol %. In some embodiments, the LNP or composition thereof comprises one or more compounds having the structure of Formula (I)-(XV) in a concentration of about 25 mol %. In some embodiments, the LNP or composition thereof comprises one or more compounds having the structure of Formula (I)-(XV) in a concentration of about 30 mol %. In some embodiments, the LNP or composition thereof comprises one or more compounds having the structure of Formula (I)-(XV) in a concentration of about 35 mol %. In some embodiments, the LNP or composition thereof comprises one or more compounds having the structure of Formula (I)-(XV) in a concentration of about 37 mol %. In some embodiments, the LNP or composition thereof comprises one or more compounds having the structure of Formula (I)-(XV) in a concentration of about 40 mol %. In some embodiments, the LNP or composition thereof comprises one or more compounds having the structure of Formula (I)-(XV) in a concentration of about 45 mol %. In some embodiments, the LNP or composition thereof comprises one or more compounds having the structure of Formula (I)-(XV) in a concentration of about 50 mol %. In some embodiments, the LNP or composition thereof comprises one or more compounds having the structure of Formula (I)-(XV) in a concentration of about 60 mol %. In some embodiments, the LNP or composition thereof comprises one or more compounds having the structure of Formula (I)-(XV) in a concentration of about 70 mol %. In some embodiments, the LNP or composition thereof comprises one or more compounds having the structure of Formula (I)-(XV) in a concentration of about 80 mol %. In some embodiments, the LNP or composition thereof comprises one or more compounds having the structure of Formula (I)-(XV) in a concentration of about 90 mol %. In some embodiments, the LNP or composition thereof comprises one or more compounds having the structure of Formula (I)-(XV) in a concentration of about 95 mol %. In some embodiments, the LNP or composition thereof comprises one or more compounds having the structure of Formula (I)-(XV) in a concentration of about 95.5 mol %. In some embodiments, the LNP or composition thereof comprises one or more compounds having the structure of Formula (I)-(XV) in a concentration of about 99 mol %. In some embodiments, the LNP or composition thereof comprises one or more compounds having the structure of Formula (I)-(XV) in a concentration of about 99.9 mol %. In some embodiments, the LNP or composition thereof comprises one or more compounds having the structure of Formula (I)-(XV) in a concentration of about 100 mol %.

In various embodiments, the LNP or composition thereof further comprises at least one helper compound. In some embodiments, the helper compound is a helper lipid, helper polymer, or any combination thereof. In some embodiments, the helper lipid is phospholipid, cholesterol lipid, polymer, cationic lipid, neutral lipid, charged lipid, steroid, steroid analogue, polymer conjugated lipid, stabilizing lipid, or any combination thereof.

In various embodiments, the LNP or composition thereof comprises one or more helper compound in a concentration range of about 0 mol % to about 100 mol %. In some embodiments, the LNP or composition thereof comprises one or more helper compound in a concentration range of about 0.01 mol % to about 99.9 mol %. In some embodiments, the LNP or composition thereof comprises one or more helper compound in a concentration range of about 0.1 mol % to about 90 mol %. In some embodiments, the LNP or composition thereof comprises one or more helper compound in a concentration range of about 0.1 mol % to about 70 mol %. In some embodiments, the LNP or composition thereof comprises one or more helper compound in a concentration range of about 5 mol % to about 95 mol %. In some embodiments, the LNP or composition thereof comprises one or more helper compound in a concentration range of about 0.5 mol % to about 50 mol %. In some embodiments, the LNP or composition thereof comprises one or more helper compound in a concentration range of about 0.5 mol % to about 47 mol %. In some embodiments, the LNP or composition thereof comprises one or more helper compound in a concentration range of about 2.5 mol % to about 47 mol %.

For example, in some embodiments, the LNP or composition thereof comprises one or more helper compound in a concentration of about 0.01 mol %. In some embodiments, the LNP or composition thereof comprises one or more helper compound in a concentration of about 0.1 mol %. In some embodiments, the LNP or composition thereof comprises one or more helper compound in a concentration of about 0.5 mol %. In some embodiments, the LNP or composition thereof comprises one or more helper compound in a concentration of about 1 mol %. In some embodiments, the LNP or composition thereof comprises one or more helper compound in a concentration of about 1.5 mol %. In some embodiments, the LNP or composition thereof comprises one or more helper compound in a concentration of about 2 mol %. In some embodiments, the LNP or composition thereof comprises one or more helper compound in a concentration of about 2.5 mol %. In some embodiments, the LNP or composition thereof comprises one or more helper compound in a concentration of about 5 mol %. In some embodiments, the LNP or composition thereof comprises one or more helper compound in a concentration of about 10 mol %. In some embodiments, the LNP or composition thereof comprises one or more helper compound in a concentration of about 12 mol %. In some embodiments, the LNP or composition thereof comprises one or more helper compound in a concentration of about 15 mol %. In some embodiments, the LNP or composition thereof comprises one or more helper compound in a concentration of about 16 mol %. In some embodiments, the LNP or composition thereof comprises one or more helper compound in a concentration of about 20 mol %. In some embodiments, the LNP or composition thereof comprises one or more helper compound in a concentration of about 25 mol %. In some embodiments, the LNP or composition thereof comprises one or more helper compound in a concentration of about 30 mol %. In some embodiments, the LNP or composition thereof comprises one or more helper compound in a concentration of about 35 mol %. In some embodiments, the LNP or composition thereof comprises one or more helper compound in a concentration of about 37 mol %. In some embodiments, the LNP or composition thereof comprises one or more helper compound in a concentration of about 40 mol %. In some embodiments, the LNP or composition thereof comprises one or more helper compound in a concentration of about 45 mol %. In some embodiments, the LNP or composition thereof comprises one or more helper compound in a concentration of about 46.5 mol %. In some embodiments, the LNP or composition thereof comprises one or more helper compound in a concentration of about 47 mol %. In some embodiments, the LNP or composition thereof comprises one or more helper compound in a concentration of about 50 mol %. In some embodiments, the LNP or composition thereof comprises one or more helper compound in a concentration of about 60 mol %. In some embodiments, the LNP or composition thereof comprises one or more helper compound in a concentration of about 63 mol %. In some embodiments, the LNP or composition thereof comprises one or more helper compound in a concentration of about 70 mol %. In some embodiments, the LNP or composition thereof comprises one or more helper compound in a concentration of about 80 mol %. In some embodiments, the LNP or composition thereof comprises one or more helper compound in a concentration of about 90 mol %. In some embodiments, the LNP or composition thereof comprises one or more helper compound in a concentration of about 95 mol %. In some embodiments, the LNP or composition thereof comprises one or more helper compound in a concentration of about 95.5 mol %. In some embodiments, the LNP or composition thereof comprises one or more helper compound in a concentration of about 99 mol %. In some embodiments, the LNP or composition thereof comprises one or more helper compound in a concentration of about 100 mol %.

In some embodiments, the phospholipid is dioleoyl-phosphatidylethanolamine (DOPE) or a derivative thereof, distearoylphosphatidylcholine (DSPC) or a derivative thereof, distearoyl-phosphatidylethanolamine (DSPE) or a derivative thereof, stearoyloleoylphosphatidylcholine (SOPC) or a derivative thereof, 1-stearioyl-2-oleoyl-phosphatidyethanol amine (SOPE) or a derivative thereof, N-(2,3-dioleoyloxy)propyl)-N,N,N-trimethylammonium chloride (DOTAP) or a derivative thereof, or any combination thereof.

For example, in some embodiments, the LNP or composition thereof comprises a phospholipid in a concentration range of about 0 mol % to about 100 mol %. In some embodiments, the LNP or composition thereof comprises a phospholipid in a concentration range of about 15 mol % to about 50 mol %. In some embodiments, the LNP or composition thereof comprises a phospholipid in a concentration range of about 10 mol % to about 40 mol %. In some embodiments, the LNP or composition thereof comprises a phospholipid in a concentration range of about 16 mol % to about 40 mol %.

In some embodiments, the cholesterol lipid is cholesterol or a derivative thereof. For example, in some embodiments, the LNP or composition thereof comprises a cholesterol lipid in a concentration range of about 0 mol % to about 100 mol %. In some embodiments, the LNP or composition thereof comprises a cholesterol lipid in a concentration range of about 20 mol % to about 50 mol %. In some embodiments, the LNP or composition thereof comprises a cholesterol lipid in a concentration range of about 20 mol % to about 47 mol %. In some embodiments, the LNP or composition thereof comprises a cholesterol lipid in a concentration of about 47 mol % and DOPE in a concentration of about 16 mol %.

In some embodiments, the polymer is polyethylene glycol (PEG) or a derivative thereof. For example, in some embodiments, the LNP or composition thereof comprises a polymer in a concentration range of about 0 mol % to about 100 mol %. In some embodiments, the LNP or composition thereof comprises a polymer in a concentration range of about 0.5 mol % to about 10 mol %. In some embodiments, the LNP or composition thereof comprises a polymer in a concentration range of about 0.5 mol % to about 2.5 mol %.

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(14(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: 20 DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), I-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt 30 (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the composition comprises a neutral lipid selected from DSPC, DPPC, DMPC, DOPC, POPC, DOPE, and SM.

A “steroid” is a compound comprising the following carbon skeleton:

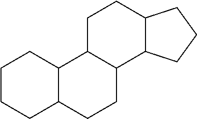 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the LNP or composition thereof comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the LNPs or compositions thereof comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(o)-methoxy(polyethoxy)ethyl)butanedioate (PEG-S-DMG), a pegylated ceramide (PEG-cer), or a PEG dialkoxypropylcarbamate such as o)-methoxy(polyethoxy)cthyl-N-(2,3-di(tetradecanoxy)propyl)carbamate or 2,3-di(tetradecanoxy)propyl-N-((o-methoxy(polyethoxy)ethyl)carbamate.

In certain embodiments, the additional lipid is present in the LNP or composition thereof in an amount from about 1 mol % to about 10 mol %. In one embodiment, the additional lipid is present in the LNP or composition thereof in an amount from about 1 mol % to about 5 mol %. In one embodiment, the additional lipid is present in the LNP or composition thereof in about 1 mol % or about 2.5 mol %.

The term “lipid nanoparticle” refers to a particle having at least one dimension on the order of nanometers (e.g., 1-1,000 nm) which includes one or more lipids, for example a lipid of Formula (I)-(XV).

In various embodiments, the LNPs have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In some embodiments, the LNPs of the present invention are substantially non-toxic. Thus, in various embodiments, the LNP compositions of the present invention are substantially non-toxic.

In various embodiments, the LNPs described herein readily transport to a target of interest. For example, in some embodiments, the LNPs described herein readily transport to a tissue of interest. In some embodiments, the LNPs described herein readily transport through a cell membrane to a cell. In some embodiments, the LNPs described herein efficiently transport through a cell membrane to a cell. In some embodiments, the LNPs described herein transport through a cell membrane to a cell with enhanced efficacy.

Thus, in various embodiments, the LNP compositions described herein readily transport to a tissue of interest. In some embodiments, the LNP compositions described herein readily transport through a cell membrane to a cell. In various embodiments, the LNP compositions described herein efficiently transport through a cell membrane to a cell. In some embodiments, the LNP compositions described herein transport through a cell membrane to a cell with enhanced efficacy.

In various aspects, the compositions of the present invention further comprise one or more nucleic acid molecules, one or more adjuvants, one or more therapeutic agents, or combination thereof. In some embodiments, the one or more nucleic acid molecules, one or more adjuvants, one or more therapeutic agents, or combination thereof are encapsulated with the compound having the structure of Formula (I). In some embodiments, the one or more nucleic acid molecules, one or more adjuvants, one or more therapeutic agents, or combination thereof are encapsulated with the compound having the structure of Formula (I)-(XV). In some embodiments, the one or more nucleic acid molecules, one or more adjuvants, one or more therapeutic agents, or combination thereof are encapsulated with the LNP.

In various embodiments, the composition comprises one or more nucleic acid molecules. In one embodiment, the nucleic acid molecule is a DNA molecule. In one embodiment, the nucleic acid molecule is a RNA molecule. In some embodiments, the nucleic acid molecule is a DNA molecule or an RNA molecule. Examples of such nucleic acid include, but are not limited to: cDNA, mRNA, miRNA, siRNA, sgRNA, modified RNA, antagomir, antisense molecule, guide RNA molecule, CRISPR guide RNA molecule, peptide, therapeutic peptide, targeted nucleic acid, and any combination thereof.

In one embodiment, the mRNA encodes a luciferase.

In various embodiments, the nucleic acid molecule is a therapeutic agent. In some embodiments, the therapeutic agent is an isolated nucleic acid. In various embodiments, the isolated nucleic acid molecule is a DNA molecule or an RNA molecule. In various embodiments, the isolated nucleic acid molecule is a cDNA, mRNA, miRNA, siRNA, antagomir, antisense molecule, or CRISPR guide RNA molecule. In one embodiment, the isolated nucleic acid molecule encodes a therapeutic peptide. In some embodiments, the therapeutic agent is an siRNA, miRNA, sgRNA or antisense molecule, which inhibits a targeted nucleic acid.

In one embodiment, the composition comprises a promoter or regulatory sequence. In one embodiment, the nucleic acid comprises a promoter or regulatory sequence such that the nucleic acid is capable of directing expression of the nucleic acid. Thus, in one embodiment, the composition comprising the metabolite-based polymer or polymeric particle of the invention comprises an expression vector, and the invention comprises a method for the introduction of exogenous DNA into cells or tissues of interest with concomitant expression of the exogenous DNA in the cells or tissues of interest.

In one embodiment, the nucleic acid molecule is an mRNA. In one embodiment, the composition comprises an mRNA. In one embodiment, the composition comprises an mRNA encapsulated within the LNP. In various embodiments, the compositions comprising mRNA encapsulated within the LNP have particular advantages over isolated mRNA, including for example, increased stability, low or absent innate immunogenicity, and enhanced translation.

In one embodiment, the mRNA encodes a chimeric antigen receptor (CAR).

In one embodiment, the RNA is a modified RNA. In another embodiment, between 0.1% and 100% of the residues in the modified of the present invention are modified. In another embodiment, 0.1% of the residues are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of the given nucleotide that is modified is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, the RNA encapsulated in the LNP of the present invention is translated in the cell more efficiently than an isolated RNA molecule with the same sequence. In another embodiment, the RNA encapsulated in the LNP exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 5-fold factor. In another embodiment, translation is enhanced by a 7-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In certain embodiments, the mRNA does not activate any pathophysiologic pathways, translates very efficiently and almost immediately following delivery, and serve as templates for continuous protein production in vivo lasting for several days (Karikó et al., 2008, Mol Ther 16:1833-1840; Karikó et al., 2012, Mol Ther 20:948-953). In certain instances, antigen encoded by mRNA encapsulated within the LNP induces greater production of antigen-specific antibody production as compared to antigen encoded by isolated mRNA.

In one embodiment, the nucleic acid molecule encodes an antigen. In one embodiment, the nucleic acid molecule encodes a plurality of antigens. In some embodiments, the mRNA encodes one or more antigens. In one embodiment, the therapeutic agent is an antigen.

In various embodiments, the antigen comprises a viral antigen, a bacterial antigen, a fungal antigen, a parasitic antigen, an influenza antigen, a tumor-associated antigen, a tumor-specific antigen, or any combination thereof. In one embodiment, the invention includes a nucleic acid molecule encoding an adjuvant.

In one embodiment, the antigen is encoded by a nucleic acid sequence of a nucleic acid molecule. In certain embodiments, the nucleic acid sequence comprises DNA, RNA, cDNA, a variant thereof, a fragment thereof, or a combination thereof. In one embodiment, the nucleic acid sequence comprises a modified nucleic acid sequence. For example, in one embodiment the antigen-encoding nucleic acid sequence comprises RNA, as described in detail elsewhere herein. In certain instances, the nucleic acid sequence comprises include additional sequences that encode linker or tag sequences that are linked to the antigen by a peptide bond.

In certain embodiments, the antigen, encoded by the nucleic acid molecule, comprises a protein, peptide, a fragment thereof, or a variant thereof, or a combination thereof from any number of organisms, for example, a virus, a parasite, a bacterium, a fungus, or a mammal. For example, in certain embodiments, the antigen is associated with an autoimmune disease, allergy, or asthma. In other embodiments, the antigen is associated with cancer, herpes, influenza, hepatitis B, hepatitis C, human papilloma virus (HPV), ebola, pneumococcus, Haemophilus influenza, meningococcus, dengue, tuberculosis, malaria, norovirus or human immunodeficiency virus (HIV). In certain embodiments, the antigen comprises a consensus sequence based on the amino acid sequence of two or more different organisms. In certain embodiments, the nucleic acid sequence encoding the antigen is optimized for effective translation in the organism in which the composition is delivered.

In one embodiment, the antigen comprises a tumor-specific antigen or tumor-associated antigen, such that the antigen induces an adaptive immune response against the tumor. In one embodiment, the antigen comprises a fragment of a tumor-specific antigen or tumor-associated antigen, such that the antigen induces an adaptive immune response against the tumor. In certain embodiment, the tumor-specific antigen or tumor-associated antigen is a mutation variant of a host protein.

Thus, in one embodiment, the composition comprises an antigen. In one embodiment, the composition comprises a nucleic acid sequence which encodes an antigen. For example, in certain embodiments, the composition comprises a RNA encoding an antigen. The antigen may be any molecule or compound, including but not limited to a polypeptide, peptide or protein that induces an adaptive immune response in a subject.

In one embodiment, the antigen comprises a polypeptide or peptide associated with a pathogen, such that the antigen induces an adaptive immune response against the antigen, and therefore the pathogen. In one embodiment, the antigen comprises a fragment of a polypeptide or peptide associated with a pathogen, such that the antigen induces an adaptive immune response against the pathogen.

In certain embodiments, the antigen comprises an amino acid sequence that is substantially homologous to the amino acid sequence of an antigen described herein and retains the immunogenic function of the original amino acid sequence. For example, in certain embodiments, the amino acid sequence of the antigen has a degree of identity with respect to the original amino acid sequence of at least 60%, advantageously of at least 70%, preferably of at least 85%, and more preferably of at least 95%.

Viral Antigens—In one embodiment, the antigen comprises a viral antigen, or fragment thereof, or variant thereof. In certain embodiments, the viral antigen is from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. In certain embodiments, the viral antigen is from papilloma viruses, for example, human papillomoa virus (HPV), human immunodeficiency virus (HIV), polio virus, hepatitis B virus, hepatitis C virus, smallpox virus (Variola major and minor), vaccinia virus, influenza virus, rhinoviruses, dengue fever virus, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus. Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, chikungunya virus, lassa virus, arenavirus, or cancer causing virus.

Hepatitis Antigen—In one embodiment, the antigen comprises a hepatitis virus antigen (i.e., hepatitis antigen), or fragment thereof, or variant thereof. In certain embodiments, the hepatitis antigen comprises an antigen or immunogen from hepatitis A virus (HAV), hepatitis B virus (HBV), hepatitis C virus (HCV), hepatitis D virus (HDV), and/or hepatitis E virus (HEV). In certain embodiments, the hepatitis antigen is full-length or immunogenic fragments of full-length proteins.

In one embodiment, the hepatitis antigen comprises an antigen from HAV. For example, in certain embodiments, the hepatitis antigen comprises a HAV capsid protein, a HAV non-structural protein, a fragment thereof, a variant thereof, or a combination thereof.

In one embodiment, the hepatitis antigen comprises an antigen from HCV. For example, in certain embodiments, the hepatitis antigen comprises a HCV nucleocapsid protein (i.e., core protein), a HCV envelope protein (e.g., E1 and E2), a HCV non-structural protein (e.g., NS1, NS2, NS3, NS4a, NS4b, NS5a, and NS5b), a fragment thereof, a variant thereof, or a combination thereof.

In one embodiment, the hepatitis antigen comprises an antigen from HDV.

For example, in certain embodiments, the hepatitis antigen comprises a HDV delta antigen, fragment thereof, or variant thereof.

In one embodiment, the hepatitis antigen comprises an antigen from HEV.

For example, in certain embodiments, the hepatitis antigen comprises a HEV capsid protein, fragment thereof, or variant thereof.

In one embodiment, the hepatitis antigen comprises an antigen from HBV.

For example, in certain embodiments, the hepatitis antigen comprises a HBV core protein, a HBV surface protein, a HBV DNA polymerase, a HBV protein encoded by gene X, fragment thereof, variant thereof, or combination thereof. In certain embodiments, the hepatitis antigen comprises a HBV genotype A core protein, a HBV genotype B core protein, a HBV genotype C core protein, a HBV genotype D core protein, a HBV genotype E core protein, a HBV genotype F core protein, a HBV genotype G core protein, a HBV genotype H core protein, a HBV genotype A surface protein, a HBV genotype B surface protein, a HBV genotype C surface protein, a HBV genotype D surface protein, a HBV genotype E surface protein, a HBV genotype F surface protein, a HBV genotype G surface protein, a HBV genotype H surface protein, fragment thereof, variant thereof, or combination thereof.

Human Papilloma Virus (HPV) Antigen—In one embodiment, the antigen comprises a human papilloma virus (HPV) antigen, or fragment thereof, or variant thereof. For example, in certain embodiments, the antigen comprises an antigen from HPV types 16, 18, 31, 33, 35, 45, 52, and 58, which cause cervical cancer, rectal cancer, and/or other cancers. In one embodiment, the antigen comprises an antigen from HPV types 6 and 11, which cause genital warts, and are known to be causes of head and neck cancer. For example, in certain embodiments, the HPV antigen comprises a HPV E6 or E7 domain, or fragments, or variant thereof from any HPV type.

RSV Antigen—In one embodiment, the antigen comprises an RSV antigen or fragment thereof, or variant thereof. For example, in certain embodiments, the RSV antigen comprises a human RSV fusion protein (also referred to herein as “RSV F”, “RSV F protein” and “F protein”), or fragment or variant thereof. In one embodiment, the human RSV fusion protein is conserved between RSV subtypes A and B. In certain embodiments, the RSV antigen comprises a RSV F protein, or fragment or variant thereof, from the RSV Long strain (GenBank AAX23994.1). In one embodiment, the RSV antigen comprises a RSV F protein from the RSV A2 strain (GenBank AAB59858.1), or a fragment or variant thereof. In certain embodiments, the RSV antigen is a monomer, a dimer or trimer of the RSV F protein, or a fragment or variant thereof. According to the invention, in certain embodiments, the RSV F protein is in a prefusion form or a postfusion form.

In one embodiment, the RSV antigen comprises a human RSV attachment glycoprotein (also referred to herein as “RSV G”, “RSV G protein” and “G protein”), or fragment or variant thereof. The human RSV G protein differs between RSV subtypes A and B. In one embodiment, the antigen comprises a RSV G protein, or fragment or variant thereof, from the RSV Long strain (GenBank AAX23993). In one embodiment, the RSV antigen comprises RSV G protein from: the RSV subtype B isolate H5601, the RSV subtype B isolate H1068, the RSV subtype B isolate H5598, the RSV subtype B isolate H1123, or a fragment or variant thereof.

In other embodiments, the RSV antigen comprises a human RSV non-structural protein 1 (“NS1 protein”), or fragment or variant thereof. For example, in one embodiment, the RSV antigen comprises RSV NS1 protein, or fragment or variant thereof, from the RSV Long strain (GenBank AAX23987.1). In one embodiment, the RSV antigen comprises RSV non-structural protein 2 (“NS2 protein”), or fragment or variant thereof. For example, in one embodiment, the RSV antigen comprises RSV NS2 protein, or fragment or variant thereof, from the RSV Long strain (GenBank AAX23988.1). In one embodiment, the RSV antigen comprises human RSV nucleocapsid (“N”) protein, or fragment or variant thereof. For example, in one embodiment, the RSV antigen is RSV N protein, or fragment or variant thereof, from the RSV Long strain (GenBank AAX23989.1). In one embodiment, the RSV antigen comprises human RSV Phosphoprotein (“P”) protein, or fragment or variant thereof. For example, in one embodiment, the RSV antigen comprises RSV P protein, or fragment or variant thereof, from the RSV Long strain (GenBank AAX23990.1). In one embodiment, the RSV antigen comprises human RSV Matrix protein (“M”) protein, or fragment or variant thereof. For example, in one embodiment, the RSV antigen comprises RSV M protein, or fragment or variant thereof, from the RSV Long strain (GenBank AAX23991.1).

In still other embodiments, the RSV antigen comprises human RSV small hydrophobic (“SH”) protein, or fragment or variant thereof. For example, in one embodiment, the RSV antigen comprises RSV SH protein, or fragment or variant thereof, from the RSV Long strain (GenBank AAX23992.1). In one embodiment, the RSV antigen comprises human RSV Matrix protein2-1 (“M2-1”) protein, or fragment or variant thereof. For example, in one embodiment, the RSV antigen comprises RSV M2-1 protein, or fragment or variant thereof, from the RSV Long strain (GenBank AAX23995.1). In one embodiment, the RSV antigen comprises RSV Matrix protein 2-2 (“M2-2”) protein, or fragment or variant thereof. For example, in one embodiment, the RSV antigen comprises RSV M2-2 protein, or fragment or variant thereof, from the RSV Long strain (GenBank AAX23997.1). In one embodiment, the RSV antigen comprises RSV Polymerase L (“L”) protein, or fragment or variant thereof. For example, in one embodiment, the RSV antigen comprises RSV L protein, or fragment or variant thereof, from the RSV Long strain (GenBank AAX23996.1).

Influenza Antigen—In one embodiment, the antigen comprises an influenza antigen or fragment thereof, or variant thereof. The influenza antigens are those capable of eliciting an adaptive immune response in a mammal against one or more influenza serotypes. In certain embodiments, the antigen comprises the full length translation product Hemagglutinin (HA)0, subunit HA1, subunit HA2, a variant thereof, a fragment thereof or a combination thereof. In certain embodiments, the influenza hemagglutinin antigen is derived from one or more strains of influenza A serotype H1, influenza A serotype H2, or influenza B.

In one embodiment, the influenza antigen contains at least one antigenic epitope that can be effective against particular influenza immunogens against which an immune response can be induced. In certain embodiments, the antigen may provide an entire repertoire of immunogenic sites and epitopes present in an intact influenza virus.

In some embodiments, the influenza antigen comprises HI HA, H2 HA, H3 HA, H5 HA, or a BHA antigen. In certain embodiments, the influenza antigen comprises neuraminidase (NA), matrix protein, nucleoprotein, M2 ectodomain-nucleo-protein (M2e-NP), a variant thereof, a fragment thereof, or combinations thereof.

Human Immunodeficiency Virus (HIV) Antigen—In one embodiment, the antigen comprises an HIV antigen or fragment thereof, or variant thereof.

In certain embodiments, the HIV antigen comprises an envelope (Env) protein or fragment or variant thereof. For example, in certain embodiments, the HIV antigen comprises an Env protein selected from gp120, gp41, or a combination thereof.

In certain embodiments, the HIV antigen comprises at least one of nef, gag, pol, vif, vpr, vpu, tat, rev, or a fragment of variant thereof.

The HIV antigen may be derived from any strain of HIV. For example, in certain embodiments the HIV antigen comprises an antigen from HIV groups M, N, O, and P, and subtype A, HIV subtype B, HIV subtype C, HIV subtype D, subtype E, subtype F, subtype G, subtype H, subtype J, or subtype K. In one embodiment, the HIV antigen comprises Env or fragment or variant thereof, from the HIV-R3A strain (R3A-Env).

Parasite Antigens—In certain embodiments, the antigen comprises a parasite antigen or fragment or variant thereof. In certain embodiments, the parasite is a protozoa, helminth, or ectoparasite. In certain embodiments, the helminth (i.e., worm) is a flatworm (e.g., flukes and tapeworms), a thorny-headed worm, or a round worm (e.g., pinworms). In certain embodiments, the ectoparasite is lice, fleas, ticks, and mites.

In certain embodiments, the parasite is any parasite causing the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

In certain embodiments, the parasite is Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus—lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

Malaria Antigen—In one embodiment, the antigen comprises a malaria antigen (i.e., PF antigen or PF immunogen), or fragment thereof, or variant thereof. For example, in one embodiment, the antigen comprises an antigen from a parasite causing malaria. In one embodiment, the malaria causing parasite is Plasmodium falciparum.

In some embodiments, the malaria antigen comprises one or more of P. falciparum immunogens CS; LSA1; TRAP; CeITOS; and Ama1. The immunogens may be full length or immunogenic fragments of full length proteins.

Bacterial Antigens—In one embodiment, the antigen comprises a bacterial antigen or fragment or variant thereof. In certain embodiments, the bacterium is from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firnicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

In certain embodiments, the bacterium is a gram positive bacterium or a gram negative bacterium. In certain embodiments, the bacterium is an aerobic bacterium or an anaerobic bacterium. In certain embodiments, the bacterium is an autotrophic bacterium or a heterotrophic bacterium. In certain embodiments, the bacterium is a mesophile, a neutrophile, an extremophile, an acidophile, an alkaliphile, a thermophile, psychrophile, halophile, or an osmophile.

In certain embodiments, the bacterium is an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. In certain embodiments, bacterium is a mycobacteria, Clostridium tetani, Yersinia pestis, Bacillus anthracis, methicillin-resistant Staphylococcus aureus (MRSA), or Clostridium difficile.

Mycobacterium tuberculosis Antigens—In one embodiment, the antigen comprises a Mycobacterium tuberculosis antigen (i.e., TB antigen or TB immunogen), or fragment thereof, or variant thereof. The TB antigen can be from the Ag85 family of TB antigens, for example, Ag85A and Ag85B. The TB antigen can be from the Esx family of TB antigens, for example, EsxA, EsxB, EsxC, EsxD, EsxE, EsxF, EsxH, EsxO, EsxQ, EsxR, EsxS, EsxT, EsxU, EsxV, and EsxW.

Fungal Antigens—In one embodiment, the antigen comprises a fungal antigen or fragment or variant thereof. In certain embodiments, the fungus is Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

Tumor Antigens—In certain embodiments, the antigen comprises a tumor antigen, including for example a tumor-associated antigen or a tumor-specific antigen. In the context of the present invention, “tumor antigen” or “hyperporoliferative disorder antigen” or “antigen associated with a hyperproliferative disorder” refer to antigens that are common to specific hyperproliferative disorders. In certain aspects, the hyperproliferative disorder antigens of the present invention are derived from cancers including, but not limited to, primary or metastatic melanoma, mesothelioma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

Tumor antigens are proteins that are produced by tumor cells that elicit an immune response, particularly T-cell mediated immune responses. In one embodiment, the tumor antigen of the present invention comprises one or more antigenic cancer epitopes immunogenically recognized by tumor infiltrating lymphocytes (TIL) derived from a cancer tumor of a mammal. The selection of the antigen will depend on the particular type of cancer to be treated or prevented by way of the composition of the invention.

Tumor antigens are well known in the art and include, for example, a glioma-associated antigen, carcinoembryonic antigen (CEA), p-human chorionic gonadotropin, alphafetoprotein (AFP), lectin-reactive AFP, thyroglobulin, RAGE-I, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, cphrinB2, CD22, insulin growth factor (IGF)-1, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-I, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68†P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In a preferred embodiment, the antigen includes but is not limited to CD19, CD20, CD22, ROR1, Mesothelin, CD33/IL3Ra, c-Met, PSMA, Glycolipid F77, EGFRvIII, GD-2, MY-ESO-1 TCR, MAGE A3 TCR, and the like.

In certain embodiments, the nucleic acid molecule encodes an antigen that induces an adaptive immune response against the antigen. In certain embodiments, the therapeutic agent is an antigen that induces an adaptive immune response against the antigen.

The nucleotide sequences encoding an antigen or adjuvant, as described herein, can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the present invention includes nucleotide sequences that are substantially homologous to the nucleotide sequences recited herein and encode an antigen or adjuvant of interest.

As used herein, a nucleotide sequence is “substantially homologous” to any of the nucleotide sequences described herein when its nucleotide sequence has a degree of identity with respect to the nucleotide sequence of at least 60%. advantageously of at least 70%, preferably of at least 85%, and more preferably of at least 95%. A nucleotide sequence that is substantially homologous to a nucleotide sequence encoding an antigen can typically be isolated from a producer organism of the antigen based on the information contained in the nucleotide sequence by means of introducing conservative or non-conservative substitutions, for example. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art.

In one embodiment, the invention relates to a construct, comprising a nucleotide sequence encoding an antigen. In one embodiment, the construct comprises a plurality of nucleotide sequences encoding a plurality of antigens. For example, in certain embodiments, the construct encodes 1 or more, 2 or more, 5 or more, 10 or more, 15 or more, or 20 or more antigens. In one embodiment, the invention relates to a construct, comprising a nucleotide sequence encoding an adjuvant. In one embodiment, the construct comprises a first nucleotide sequence encoding an antigen and a second nucleotide sequence encoding an adjuvant.

In one embodiment, the composition comprises a plurality of constructs, each construct encoding one or more antigens. In certain embodiments, the composition comprises 1 or more, 2 or more, 5 or more, 10 or more, 15 or more, or 20 or more constructs. In one embodiment, the composition comprises a first construct, comprising a nucleotide sequence encoding an antigen; and a second construct, comprising a nucleotide sequence encoding an adjuvant.

In another particular embodiment, the construct is operatively bound to a translational control element. The construct can incorporate an operatively bound regulatory sequence for the expression of the nucleotide sequence of the invention, thus forming an expression cassette.

In one embodiment, the composition of the invention comprises in vitro transcribed (IVT) RNA. For example, in certain embodiments, the composition of the invention comprises IVT RNA which encodes an antigen, where the antigen induces an adaptive immune response. In certain embodiments, the antigen is at least one of a viral antigen, bacterial antigen, fungal antigen, parasitic antigen, tumor-specific antigen, or tumor-associated antigen. However, the present invention is not limited to any particular antigen or combination of antigens.

For example, in one embodiment, the composition comprises an antigen-encoding nucleic acid molecule encapsulated within a LNP. In certain instances, the LNP enhances cellular uptake of the nucleic acid molecule.

In one aspect, the nucleic acid molecule is an IVT RNA encoding an antigen. Thus, in one embodiment, the composition of the present invention comprises an IVT RNA encoding an antigen. In one embodiment, the composition of the invention comprises an IVT RNA encoding a plurality of antigens. In one embodiment, the composition of the invention comprises an IVT RNA encoding an adjuvant. In one embodiment, the composition of the invention comprises an IVT RNA encoding one or more antigens and one or more adjuvants.

In one embodiment, the composition comprises a nucleic acid molecule encoding an adjuvant. Thus, in one embodiment, the composition comprises an adjuvant. In one embodiment, the adjuvant-encoding nucleic acid molecule is IVT RNA. In one embodiment, the adjuvant-encoding nucleic acid molecule is RNA.

Exemplary adjuvants include, but is not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, pl50.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R₂, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R₃, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

In some embodiments, the composition further comprises a cationic lipid and one or more excipient selected from neutral lipids, charged lipids, steroids and polymer conjugated lipids (e.g., a pegylated lipid). In some embodiments, the nucleic acid molecule is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In one embodiment, the composition comprises one or more cationic lipids, and one or more stabilizing lipids. Stabilizing lipids include neutral lipids and pegylated lipids.

In one embodiment, the composition comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In certain embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium 15 trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane 20 (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In certain embodiments, the cationic lipid is present in the composition in an amount from about 30 to about 95 mole percent. In one embodiment, the cationic lipid is present in the composition in an amount from about 30 to about 70 mole percent. In one embodiment, the cationic lipid is present in the composition in an amount from about 40 to about 60 mole percent. In one embodiment, the cationic lipid is present in the composition in an amount of about 50 mole percent. In one embodiment, the composition comprises only cationic lipids.

In certain embodiments, the composition comprises one or more additional lipids which stabilize the formation of particles during their formation. Suitable stabilizing lipids include neutral lipids and anionic lipids.

The term “neutral lipid” refers to any one of a number of lipid species that exist in either an uncharged or neutral zwitterionic form at physiological pH.

Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). and SM. In one embodiment, the neutral lipid is 1,2-distearoyl-sn-glycero-3-phosphocholine (DSPC).

In some embodiments, the composition comprises a neutral lipid selected from DSPC, DPPC, DMPC, DOPC, POPC, DOPE and SM.

In various embodiments, the composition further comprises a steroid or steroid analogue. A “steroid” is a compound comprising the following carbon skeleton:

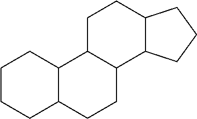 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In certain embodiments, the composition comprises glycolipids (e.g., monosialoganglioside GM₁). In certain embodiments, the composition comprises a sterol, such as cholesterol.

In some embodiments, the composition comprises a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the composition comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(u)-methoxy(polyethoxy)ethyl)butanedioate (PEG-S-DMG), a pegylated ceramide (PEG-cer), or a PEG dialkoxypropylcarbamate such as Co-methoxy(polyethoxy)ethyl-N-(2,3-di(tetradecanoxy)propyl)carbamate or 2,3-di(tetradecanoxy)propyl-N-(o)-methoxy(polyethoxy)ethyl)carbamate. In various embodiments, the molar ratio of the cationic lipid to the pegylated lipid ranges from about 100:1 to about 25:1.

In certain embodiments, the additional lipid is present in the LNP in an amount from about 1 to about 10 mole percent. In one embodiment, the additional lipid is present in the LNP in an amount from about 1 to about 5 mole percent. In one embodiment, the additional lipid is present in the LNP in about 1 mole percent or about 1.5 mole percent.

In certain embodiments, the nucleic acid molecule, when present in the lipid nanoparticles, is resistant in aqueous solution to degradation with a nuclease.

In various embodiments, the composition comprises one or more transfection reagent. In another embodiment, the transfection reagent is a lipid-based transfection reagent. In another embodiment, the transfection reagent is a protein-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity. In another embodiment, liposomes are hollow spherical vesicles composed of lipids arranged in a similar fashion as those lipids which make up the cell membrane. They have, in another embodiment, an internal aqueous space for entrapping water-soluble compounds and range in size from 0.05 to several microns in diameter. In another embodiment, liposomes can deliver RNA to cells in a biologically active form.

In various embodiments, the compositions of the present invention may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In certain embodiments, the composition comprises one or more targeting moieties which are capable of targeting the LNP to a cell, cell population, tissue of interest, or any combination thereof. For example, in one embodiment, the targeting moiety is a ligand which directs the LNP to a receptor found on a cell surface.

In certain embodiments, the composition comprises one or more internalization domains. For example, in one embodiment, the composition comprises one or more domains which bind to a cell to induce the internalization of the LNP. For example, in one embodiment, the one or more internalization domains bind to a receptor found on a cell surface to induce receptor-mediated uptake of the LNP. In certain embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo (e.g., one or more nucleic acid molecules, one or more therapeutic agents, or any combination thereof).

The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In one embodiment, the desired template for in vitro transcription is an antigen capable of inducing an adaptive immune response, including for example an antigen associated with a pathogen or tumor, as described elsewhere herein. In one embodiment, the desired template for in vitro transcription is an adjuvant capable of enhancing an adaptive immune response.

In one embodiment, the DNA to be used for PCR contains an open reading frame. The DNA can be from a naturally occurring DNA sequence from the genome of an organism. In one embodiment, the DNA is a full length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. In another embodiment, the DNA to be used for PCR is a gene from a pathogenic or commensal organism, including bacteria, viruses, parasites, and fungi. In another embodiment, the DNA to be used for PCR is from a pathogenic or commensal organism, including bacteria, viruses, parasites, and fungi, including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that induce or enhance an adaptive immune response in an organism. Preferred genes are genes which are useful for a short term treatment, or where there are safety concerns regarding dosage or the expressed gene.

In various embodiments, a plasmid is used to generate a template for in vitro transcription of mRNA which is used for transfection.

Chemical structures with the ability to promote stability and/or translation efficiency may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to mRNA molecules. In a preferred embodiment, RNAs produced by the methods to include a 5′ cap1 structure. Such cap1 structure can be generated using Vaccinia capping enzyme and 2′-O-methyltransferase enzymes (CellScript, Madison, Wis.). Alternatively, 5′ cap is provided using techniques known in the art and described herein (Cougot, et al., Trends in Biochem. Sci., 29:436-444 (2001). Stepinski, et al., RNA, 7:1468-95 (2001); Elango, et al., Biochim. Biophys. Res. Commun., 330:958-966 (2005)).

The nucleic acid sequences coding for the antigen or adjuvant can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, sequencing vectors and vectors optimized for in vitro transcription.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/RNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Chol”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### LNP Vaccine

In one aspect of the invention, the compositions described herein are vaccines. For a composition to be useful as a vaccine, the composition must induce an adaptive immune response to the antigen in a cell, tissue, or mammal (e.g., a human). In certain instances, the vaccine induces a protective immune response in the mammal. As used herein, an “immunogenic composition” may comprise an antigen (e.g., a peptide or polypeptide), a nucleic acid encoding an antigen, a cell expressing or presenting an antigen or cellular component, or a combination thereof. In particular embodiments the composition comprises or encodes all or part of any peptide antigen described herein, or an immunogenically functional equivalent thereof. In other embodiments, the composition is in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

In the context of the present invention, the term “vaccine” refers to a substance that induces immunity upon inoculation into animals.

A vaccine of the present invention may vary in its composition of nucleic acid and/or cellular components. In a non-limiting example, a nucleic acid encoding an antigen might also be formulated with an adjuvant. Of course, it will be understood that various compositions described herein may further comprise additional components. For example, one or more vaccine components may be comprised in a lipid, liposome, or lipid nanoparticle. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

The induction of the immunity by the expression of the antigen can be detected by observing in vivo or in vitro the response of all or any part of the immune system in the host against the antigen.

For example, a method for detecting the induction of cytotoxic T lymphocytes is well known. A foreign substance that enters the living body is presented to T cells and B cells by the action of APCs. T cells that respond to the antigen presented by APC in an antigen specific manner differentiate into cytotoxic T cells (also referred to as cytotoxic T lymphocytes or CTLs) due to stimulation by the antigen. These antigen stimulated cells then proliferate. This process is referred to herein as “activation” of T cells. Therefore, CTL induction by an epitope of a polypeptide or peptide or combinations thereof can be evaluated by presenting an epitope of a polypeptide or peptide or combinations thereof to a T cell by APC, and detecting the induction of CTL. Furthermore, APCs have the effect of activating B cells, CD4+ T cells, CD8+ T cells, macrophages, eosinophils and NK cells.

A method for evaluating the inducing action of CTL using dendritic cells (DCs) as APC is well known in the art. DC is a representative APC having a robust CTL inducing action among APCs. In the methods of the invention, the epitope of a polypeptide or peptide or combinations thereof is initially expressed by the DC and then this DC is contacted with T cells. Detection of T cells having cytotoxic effects against the cells of interest after the contact with DC shows that the epitope of a polypeptide or peptide or combinations thereof has an activity of inducing the cytotoxic T cells. Furthermore, the induced immune response can be also examined by measuring IFN-gamma produced and released by CTL in the presence of antigen-presenting cells that carry immobilized peptide or combination of peptides by visualizing using anti-IFN-gamma antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7.

The antigens confirmed to possess CTL-inducing activity by these methods are antigens having DC activation effect and subsequent CTL-inducing activity. Furthermore, CTLs that have acquired cytotoxicity due to presentation of the antigen by APC can be also used as vaccines against antigen-associated disorders.

The induction of immunity by expression of the antigen can be further confirmed by observing the induction of antibody production against the antigen. For example, when antibodies against an antigen are induced in a laboratory animal immunized with the composition encoding the antigen, and when antigen-associated pathology is suppressed by those antibodies, the composition is determined to induce immunity.

The induction of immunity by expression of the antigen can be further confirmed by observing the induction of CD4+ T cells. CD4+ T cells can also lyse target cells, but mainly supply help in the induction of other types of immune responses, including CTL and antibody generation. The type of CD4+ T cell help can be characterized, as Th1, Th2, Th9, Th17, Tregulatory, or T follicular helper (T_{fh}) cells. Each subtype of CD4+ T cell supplies help to certain types of immune responses. Of particular interest to this invention, the T_{fh} subtype provides help in the generation of high affinity antibodies.

### Pharmaceutical LNP Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, subcutaneous, intraventricular, intrathecal, intratracheal, intraperitoneal, in utero delivery, or another route of administration or any combination thereof. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In some embodiments, parenteral administration is contemplated to include, but is not limited to, intraocular, intravitreal, subcutaneous, intraperitoneal, in utero delivery, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

The therapeutic compounds or compositions of the invention may be administered prophylactically (i.e., to prevent disease or disorder) or therapeutically (i.e., to treat disease or disorder) to subjects suffering from or at risk of (or susceptible to) developing the disease or disorder. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

### Methods of Delivery

In one aspect, the present invention provides a method for delivery of an mRNA molecule encoding a CAR, nucleic acid molecule, therapeutic agent, or any combination thereof to a target of interest. Examples of such targets include, but are not limited to, an immune cell, T cell, resident T cells, B cell, natural killer (NK) cell, cancerous cell, cell associated with a disease or disorder, tissue associated with a disease or disorder, brain tissue, central nervous system tissue, pulmonary tissue, apical surface tissue, epithelial cell, endothelial cell, liver tissue, intestine tissue, colon tissue, small intestine tissue, large intestine tissue, feces, bone marrow, macrophages, spleen tissue, muscles tissue, joint tissue, tumor cell, diseased tissue, lymph node tissue, lymphatic circulation, or any combination thereof.

In various embodiments, the method comprises administering a therapeutically effectively amount of at least one LNP or composition of the present invention. For example, in some embodiments, the method comprises the LNP or the composition thereof of the present invention delivering the mRNA molecule encoding CAR, a nucleic acid molecule, therapeutic agent, or any combination thereof to a cell. Examples of such cells include, but are not limited to, T cell, resident T cells, B cell, natural killer (NK) cell, cancerous cell, cell associated with a disease or disorder, epithelial cell, endothelial cell, tumor cell, or any combination thereof.

In one aspect, the present invention also discloses a method for delivery of an mRNA molecule encoding CAR to a subject in need thereof. In various embodiments, the method comprises administering a therapeutically effectively amount of at least one LNP or composition thereof of the present invention to the subject.

In one aspect, the present invention also discloses a method for delivery of an mRNA molecule encoding a CAR, as well as a nucleic acid molecule, adjuvant, and/or therapeutic agent to a subject in need thereof. In various embodiments, the method comprises administering a therapeutically effectively amount of at least one LNP or composition thereof of the present invention to the subject. In various embodiments, the method comprises the LNP or composition thereof of the present invention delivering the mRNA molecule as well as the nucleic acid molecule, adjuvant, and/or therapeutic agent to the subject's cell, tissue, or both.

In one aspect, the method is a gene delivery method.

In one embodiment, the method comprises IVT RNA described herein that can be introduced to a target of interest (e.g., cell, tissue, etc.) as a form of transient transfection using the LNP compositions of the present invention.

In one embodiment, the method comprises a single administration of the composition. In one embodiment, the method comprises multiple administrations of the composition.

In some embodiments, the composition is administered by an intradermal delivery route, subcutaneous delivery route, intramuscular delivery route, intraventricular delivery route, intrathecal delivery route, oral delivery route, intravenous delivery route, intratracheal delivery route, intraperitoneal delivery route, in utero delivery route, or any combination thereof.

In some embodiments, the methods for delivery of an mRNA molecule encoding a CAR, nucleic acid molecule, therapeutic agent, or any combination thereof to a target of interest (e.g., cell, tissue, etc.) comprising administering a therapeutically effectively amount of at least one LNP or composition thereof of the present invention is concurrently performed with any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser 11 (BioRad, Denver, Colo.), Multiporator (Eppendort, Hamburg Germany), TransIT®-mRNA transfection Kit (Mirus, Madison Wis.), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 min of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In order to confirm the presence of the mRNA sequence in the host cell, a variety of assays may be performed. Such assays include, for example, “molecular biological” assays well known to those of skill in the art, such as Northern blotting and RT-PCR; “biochemical” assays, such as detecting the presence or absence of a particular peptide, e.g., by immunogenic means (ELISAs and Western blots) or by assays described herein to identify agents falling within the scope of the invention.

### Treatment Methods

The present invention provides methods of inducing an adaptive immune response in a subject comprising administering an effective amount of at least one LNP or a composition thereof of the present invention comprising one or more mRNA molecules encoding CAR. In various aspects, the present invention provides methods of inducing an adaptive immune response in a subject comprising administering an effective amount of at least one LNP composition comprising one or more LNP and one or more mRNA molecules encoding CAR. In some aspects, the composition further comprises a nucleic acid molecule, therapeutic agent, or any combination thereof. In some embodiments, the nucleic acid molecule encodes one or more antigens.

In one embodiment, the method provides immunity in the subject to an infection, cancer, or disease or disorder associated with an antigen. The present invention thus provides a method of treating or preventing the infection, cancer, or disease, or disorder associated with the antigen. Exemplary antigens and associated infections, diseases, and tumors are described elsewhere herein.

For example, the method may be used to treat or prevent a viral infection, bacterial infection, fungal infection, parasitic infection, arthritis, heart disease, cardiovascular disease, neurological disorder or disease, genetic disease, autoimmune disease, fetal disease, genetic disease affecting fetal development, or cancer, depending upon the type of antigen of the administered composition.

The following are non-limiting examples of cancers that can be treated by the disclosed methods and compositions: acute lymphoblastic; acute myeloid leukemia; adrenocortical carcinoma; adrenocortical carcinoma, childhood; appendix cancer; basal cell carcinoma; bile duct cancer, extrahepatic; bladder cancer; bone cancer; osteosarcoma and malignant fibrous histiocytoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytotna/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer, craniopharyngioma; cutaneous T-cell lymphoma; esophageal cancer; Ewing family of tumors; extragonadal germ cell tumor, extrahepatic bile duct cancer, eye cancer, intraocular melanoma; eye cancer, retinoblastoma; gallbladder cancer, gastric (stomach) cancer; gastrointestinal carcinoid tumor, gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, langerhans cell; Hodgkin lymphoma; hypopharyngeal cancer, hypothalamic and visual pathway glioma; intraocular melanoma; islet cell tumors; kidney (renal cell) cancer; Langerhans cell histiocytosis; laryngeal cancer, leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, burkitt; lymphoma, cutaneous T-cell; lymphoma, non-Hodgkin lymphoma; lymphoma, primary central nervous system; macroglobulinemia, Waldenstrom; malignant fibrous histiocvtoma of bone and osteosarcoma; medulloblastoma; melanoma; melanoma, intraocular (eye); Merkel cell carcinoma; mesothelioma; metastatic squamous neck cancer with occult primary; mouth cancer, multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor, ovarian low malignant potential tumor; pancreatic cancer, pancreatic cancer, islet cell tumors; papillomatosis; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor, plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer, sarcoma, ewing family of tumors; sarcoma, Kaposi; sarcoma, soft tissue; sarcoma. uterine; sezary syndrome; skin cancer (nonmelanoma); skin cancer (melanoma); skin carcinoma, Merkel cell; small cell lung cancer; small intestine cancer, soft tissue sarcoma; squamous cell carcinoma, squamous neck cancer with occult primary, metastatic; stomach (gastric) cancer; supratentorial primitive neuroectodermal tumors; T-cell lymphoma, cutaneous; testicular cancer; throat cancer. thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer, vulvar cancer; Waldenstrom macroglobulinemia; and Wilms tumor.

In one embodiment, the composition is administered to a subject having an infection, disease, heart disease, cardiovascular disease, neurological disorder or disease, genetic disease, autoimmune disease, or cancer associated with the antigen. In one embodiment, the composition is administered to a subject at risk for developing the infection, disease, heart disease, cardiovascular disease, neurological disorder or disease, genetic disease, autoimmune disease, or cancer associated with the antigen. For example, the composition may be administered to a subject who is at risk for being in contact with a virus, bacteria, fungus, parasite, or the like. In one embodiment, the composition is administered to a subject who has increased likelihood, though genetic factors, environmental factors, or the like, of developing cancer.

In some embodiments, the composition is administered by an intradermal delivery route, subcutaneous delivery route, intramuscular delivery route, intraventricular delivery route, intrathecal delivery route, oral delivery route, intravenous delivery route, intratracheal delivery route, intraperitoneal delivery route, in utero delivery route, or any combination thereof.

In another embodiment, the composition of the present invention, comprising an antigen-encoding RNA, induces significantly more adaptive immune response than an unmodified in vitro-synthesized RNA molecule with the same sequence. In another embodiment, the composition exhibits an adaptive immune response that is 2-fold greater than its unmodified counterpart. In another embodiment, the adaptive immune response is increased by a 3-fold factor. In another embodiment the adaptive immune response is increased by a 5-fold factor. In another embodiment, the adaptive immune response is increased by a 7-fold factor. In another embodiment, the adaptive immune response is increased by a 10-fold factor. In another embodiment, the adaptive immune response is increased by a 15-fold factor. In another embodiment the adaptive immune response is increased by a 20-fold factor. In another embodiment, the adaptive immune response is increased by a 50-fold factor. In another embodiment, the adaptive immune response is increased by a 100-fold factor. In another embodiment, the adaptive immune response is increased by a 200-fold factor. In another embodiment, the adaptive immune response is increased by a 500-fold factor. In another embodiment, the adaptive immune response is increased by a 1000-fold factor. In another embodiment, the adaptive immune response is increased by a 2000-fold factor. In another embodiment, the adaptive immune response is increased by another fold difference.

In another embodiment, “induces significantly more adaptive immune response” refers to a detectable increase in an adaptive immune response. In another embodiment, the term refers to a fold increase in the adaptive immune response (e.g., 1 of the fold increases enumerated above). In another embodiment, the term refers to an increase such that the composition of the present invention, comprising a RNA, can be administered at a lower dose or frequency than an isolated RNA molecule with the same species while still inducing an effective adaptive immune response. In another embodiment, the increase is such that the composition of the present invention, comprising a RNA, can be administered using a single dose to induce an effective adaptive immune response.

In another embodiment, the composition of the present invention, comprising a RNA, exhibits significantly less innate immunogenicity than an isolated in vitro-synthesized RNA molecule with the same sequence. In another embodiment, the composition of the present invention, comprising a RNA, exhibits an innate immune response that is 2-fold less than its isolated counterpart. In another embodiment, innate immunogenicity is reduced by a 3-fold factor. In another embodiment, innate immunogenicity is reduced by a 5-fold factor. In another embodiment, innate immunogenicity is reduced by a 7-fold factor. In another embodiment, innate immunogenicity is reduced by a 10-fold factor. In another embodiment, innate immunogenicity is reduced by a 15-fold factor. In another embodiment, innate immunogenicity is reduced by a 20-fold factor. In another embodiment, innate immunogenicity is reduced by a 50-fold factor. In another embodiment, innate immunogenicity is reduced by a 100-fold factor. In another embodiment, innate immunogenicity is reduced by a 200-fold factor. In another embodiment, innate immunogenicity is reduced by a 500-fold factor. In another embodiment, innate immunogenicity is reduced by a 1000-fold factor. In another embodiment, innate immunogenicity is reduced by a 2000-fold factor. In another embodiment, innate immunogenicity is reduced by another fold difference.

In another embodiment, “exhibits significantly less innate immunogenicity” refers to a detectable decrease in innate immunogenicity. In another embodiment, the term refers to a fold decrease in innate immunogenicity (e.g., 1 of the fold decreases enumerated above). In another embodiment, the term refers to a decrease such that an effective amount of the composition of the present invention, comprising a RNA, can be administered without triggering a detectable innate immune response. In another embodiment, the term refers to a decrease such that the composition of the present invention, comprising a RNA, can be repeatedly administered without eliciting an innate immune response sufficient to detectably reduce production of the recombinant protein. In another embodiment, the decrease is such that the composition of the present invention, comprising a RNA, can be repeatedly administered without eliciting an innate immune response sufficient to eliminate detectable production of the recombinant protein.

In one aspect, the present invention related, in part, to methods of preventing or treating a disease or disorder in a subject in need thereof. In various embodiments, the method comprises administering a therapeutically effectively amount of at least one LNP or a composition thereof of the present invention to the subject. In some embodiments, the composition delivers a nucleic acid molecule, therapeutic agent, or a combination thereof to a target of interest (e.g., cell, tissue, etc.).

In one embodiment, the method comprises administering a composition comprising one or more nucleic acid molecules encoding one or more antigens and one or more adjuvant. In one embodiment, the method comprises administering a composition comprising a first nucleic acid molecule encoding one or more antigens and a second nucleic acid molecule encoding one or more adjuvants. In one embodiment, the method comprises administering a first composition comprising one or more nucleic acid molecules encoding one or more antigens and administering a second composition comprising one or more nucleic acid molecules encoding one or more adjuvants.

In certain embodiments, the method comprises administering to subject a plurality of nucleic acid molecules encoding a plurality of antigens, adjuvants, or a combination thereof.

In certain embodiments, the method of the invention allows for sustained expression of the antigen or adjuvant, described herein, for at least several days following administration. However, the method, in certain embodiments, also provides for transient expression, as in certain embodiments, the nucleic acid is not integrated into the subject genome.

In certain embodiments, the method comprises administering RNA which provides stable expression of the antigen or adjuvant described herein. In some embodiments, administration of RNA results in little to no innate immune response, while inducing an effective adaptive immune response.

Administration of the compositions of the invention in a method of treatment can be achieved in a number of different ways, using methods known in the art. In one embodiment, the method of the invention comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

It will be appreciated that the composition of the invention may be administered to a subject either alone, or in conjunction with another agent.

The therapeutic and prophylactic methods of the invention thus encompass the use of pharmaceutical compositions encoding an antigen, adjuvant, or a combination thereof. described herein to practice the methods of the invention. The pharmaceutical compositions useful for practicing the invention may be administered to deliver a dose of from ng/kg/day and 100 mg/kg/day. In one embodiment, the invention envisions administration of a dose which results in a concentration of the compound of the present invention from 10 nM and 10 μM in a mammal.

Typically, dosages which may be administered in a method of the invention to a mammal, preferably a human, range in amount from 0.01 pg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 0.1 pg to about 10 mg per kilogram of body weight of the mammal. More preferably, the dosage will vary from about 1 pg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of the composition or vaccine of the present invention may be performed by single administration or boosted by multiple administrations.

In one embodiment, the invention includes a method comprising administering one or more compositions encoding one or more antigens or adjuvants described herein. In certain embodiments, the method has an additive effect, wherein the overall effect of the administering the combination is approximately equal to the sum of the effects of administering each antigen or adjuvant. In other embodiments, the method has a synergistic effect, wherein the overall effect of administering the combination is greater than the sum of the effects of administering each antigen or adjuvant.

In one embodiment, the method comprises the systemic administration of the composition into the subject, including for example intradermal administration. In certain embodiments, the method comprises administering a plurality of doses to the subject. In another embodiment, the method comprises administering a single dose of the composition, where the single dose is effective in inducing an adaptive immune response.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Ionizable Lipid Nanoparticles for mRNA-Based T Cell Engineering

Nanoparticle (NP)-based delivery systems, comprised of lipid- and polymer-based materials, offer a promising means to overcomes the challenges faced using mechanical and viral cell engineering methods (DiTommaso T et al., 2018, PNAS, 115; Hajj K A et al., 2017, Nat Rev Mater, 2; McKinlay C J et al., 2018, PNAS, 115:E5859-E5866, Mukalel A J et al., 2019, Cancer Lett, 458:102-112). NPs have numerous potential benefits including the ability to stabilize nucleic acid cargo, aid in intracellular delivery, and mitigate toxicity (Pardi N et al., 2018, Nat Rev Drug Discov, 17:261-279; Fornaguera C et al., 2018, Adv Healthc Mater, 7:1-11; Zhang R et al., 2018, J Control Release, 292:256-276; Islam M A et al., 2015, Biomater Sci, 1519-1533). There have been some investigations into polymer-based NPs for mRNA delivery to cells with promising results, including reduced toxicity compared to EP (McKinlay C J et al., 2018, PNAS, 115:E5859-E5866; Olden B R et al., 2018, J Control Release, 282:140-147; Moffett H F et al., 2017, Nat Commun, 8:389; Démoulins T et al., 2016, Biol Med, 12:711-722; Anderson D G et al., 2003, Angew Chem Int Ed Engl, 42:3153-3158).

However, ionizable lipid nanoparticle (LNP) delivery systems are more clinically advanced than polymers in the context of RNA delivery given the approval of Alnylam's Onpattro (Pardi N et al., 2018, Nat Rev Drug Discov, 17:261-279; Garber K et al., 2018, Nat Biotechnol, 36:777). Additionally, LNPs have an ionizable lipid core that remains neutral in a physiologically relevant pH but builds charge in acidic environments, such as the endosome, to ultimately aid endosomal escape and cause 25 potent intracellular nucleic acid delivery (Hajj K A et al., 2017, Nat Rev Mater, 2; Kauffman K J et al., 2016, J Control Release, 240:227-234; Oberli M A et al., 2017, Nano Lett, 17:1326-1335; Fan Y N et al., 2018, Biomater Sci Royal Society of Chemistry, 6:3009-3018). This has been validated across a variety of cell types, including immune cells, with minimal toxicity, and previous work on lymphocyte delivery revealed that 30 LNPs deliver mRNA more effectively than commercially available lipofectamine (Hajj K A et al., 2017, Nat Rev Mater, 2; McKinlay C J et al., 2018, PNAS, 115:E5859-E5866; Zhang R et al., 2018, J Control Release, 292:256-276; Kauffman K J et al., 2016, J Control Release, 240:227-234; Oberli M A et al., 2017, Nano Lett, 17:1326-1335; Love K T et al., 2010, Proc Natl Acad Sci, 107:9915-9915).

Further, the easily modifiable composition of LNPs allows for the adjustment of their physicochemical properties to maximize their uptake into specific cell types while their ionizable properties allow them to electrostatically complex with negatively charged nucleic acid cargo (Hajj K A et al., 2017, Nat Rev Mater, 2; McKinlay C J et al., 2018, PNAS, 115:E5859-E5866; Zhang R et al., 2018, J Control Release, 292:256-276; Kauffman K J et al., 2016, J Control Release, 240:227-234; Love K T et al., 2010, Proc Natl Acad Sci, 107:9915-9915; Kauffman K J et al., 2015, Nano Lett, 15:7300-7306). These properties make LNPs an ideal platform for this investigation into the development of a LNP platform for human CAR T cell engineering.

The studies described herein utilize the novel ionizable lipid C14-4 to generate C14-4 lipid nanoparticles (LNPs) to transfect T cells. The data shows that using C14-4 LNPs allows for the same potency of T cell transfection with lower toxicity compared to EP. Additionally, the CAR T cells made with C14-4 LNPs functioned identically to EP-generated CAR T cells in terms of cancer cell killing. Further advantages of C14-4 LNPs include that they have the potential to be applied to in vivo T cell engineering to avoid patient T cell harvesting and reinfusion, and that they have already shown the ability to be further optimized (by modifying the C14-4 LNP formulation parameters) to enhance mRNA delivery.

C14-4 LNPs can provide an entirely new way to engineer T cells in both clinical manufacturing settings, such as for generating CAR T cells and research settings. Because C14-4 was able to enhance mRNA delivery when compared to lipofectamine (a gold-standard transfection reagent in cell-based research), it can be used to study T cells beyond CAR applications.

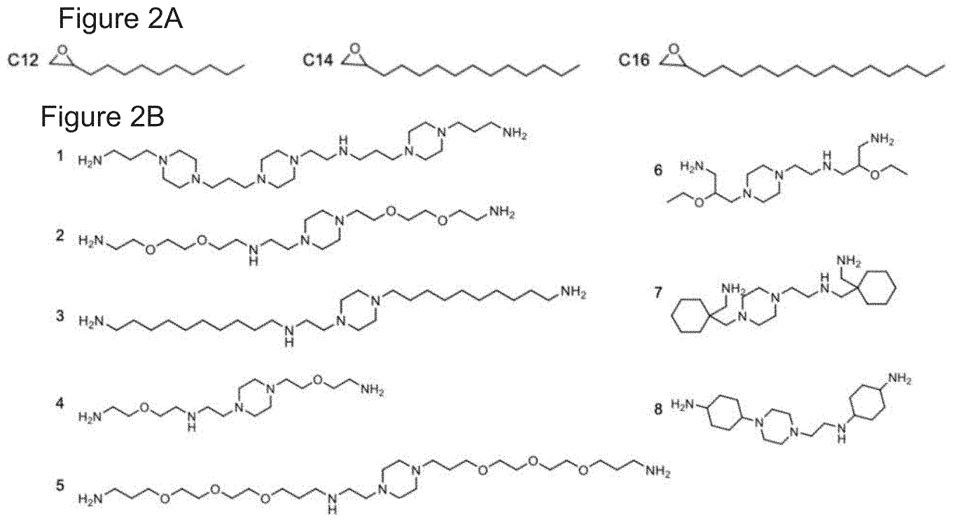

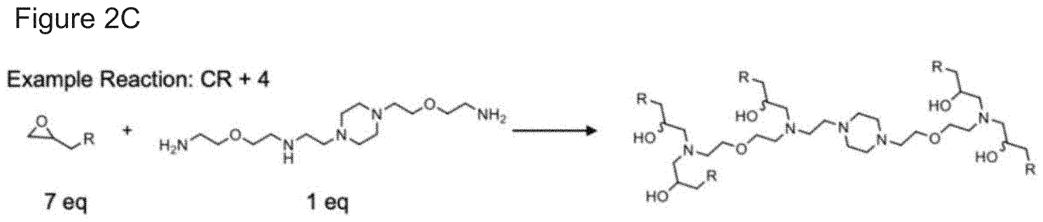

More specifically, a diverse library of 24 LNPs was generated (FIG. 2A), characterized (FIG. 2B), and screened for luciferase mRNA delivery to Jurkat cells, an immortalized human T cell line. The ionizable lipids were first synthesized via Michael addition chemistry, where polyamine cores were reacted with an excess of epoxide-terminated alkyl chains of varying lengths (FIG. 2C). Here, the lipids were evaluated for mRNA delivery to T cells.

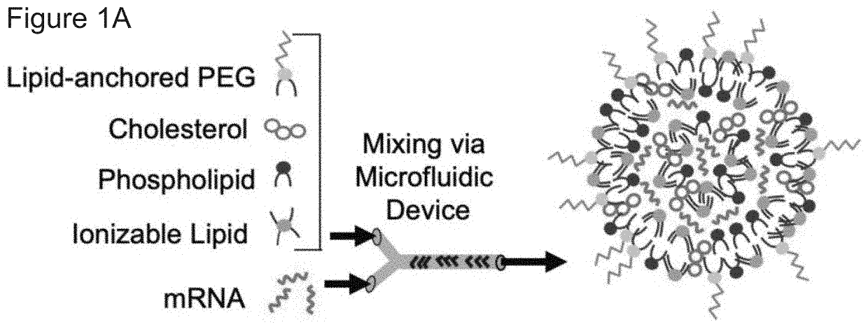

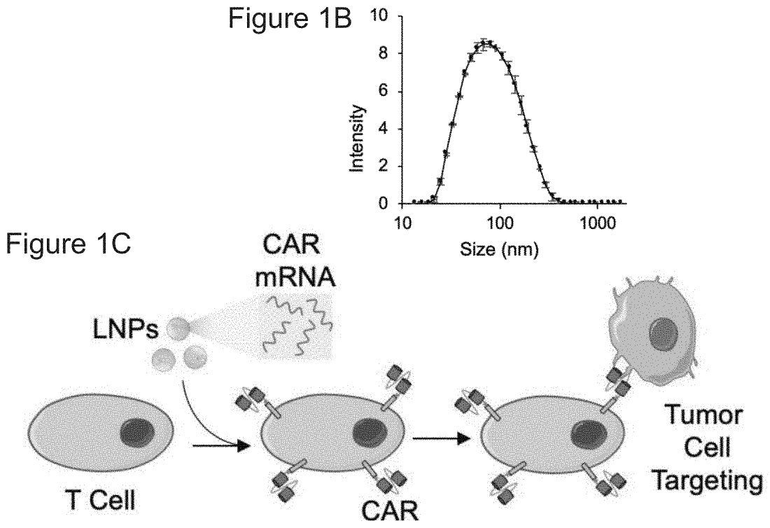

To formulate LNPs, ionizable lipids were combined in ethanol with three other excipients: (i) cholesterol for LNP stability and membrane fusion, (ii) 1,2-distearoyl-sn-glycero-3-phosphoe-thanolamine (DOPE) to fortify the bilayer structure of the LNP and promote endosomal escape, and (iii) C14-PEG to reduce aggregation and nonspecific endocytosis (Granot Y et al., 2017, Semin Immunol, 34:68-77; Varkouhi A K et al., 2011, J Control Release, 151:220-228; Mui B L et al., 2013, Mol Ther Acids, 2:1-8). This ethanol phase was then mixed with aqueous phase mRNA in a microfluidic device (FIG. 1). These excipients and their molar ratios were chosen based off of previously optimized LNP formulations for mRNA delivery, which generally utilized (i) DOPE as the phospholipid component, (ii) a decreased molar percentage of ionizable lipid, and (iii) increased concentrations of cholesterol and lipid-PEG (Kauffman K J et al., 2015, Nano Lett, 15: 300-7306; Ball R L e al., 2018, Nano Lett, 18:3814-3822). Given that alterations in the molar ratio of excipients impact the physicochemical properties and ultimately potent delivery of LNPs, the ratio of the components was held constant throughout these experiments (Kauffman K J et al., 2015, Nano Lett, 15: 300-7306; Ball R L e al., 2018, Nano Lett, 18:3814-3822; Cheng Q et al., 2018, Adv Mater, 30:1805308).

To evaluate the LNPs for their ability to deliver functional mRNA, luciferase was chosen as the encoded reporter protein. This screen revealed seven LNP formulations that enhanced mRNA delivery compared to lipofectamine, a commonly used transfection reagent (Cardarelli F et al., 2016, Sci Reports, 6:25879). Further, upon screening of 24 LNPs for mRNA delivery to Jurkat cells (immortalized human T cells), a 25 top LNP formulation, C14-4 LNPs was selected for further development for its potent delivery and low toxicity. C14-4 LNPs were then optimized for the transfection of primary T cells, and it was shown that purification of the saturated ionizable lipid led to improved mRNA delivery over the crude product. To illustrate the translatability of the platform, the optimized C14-4 LNPs were used to encapsulate CAR mRNA to generate CAR T cells (FIG. 1C). In comparison to EP-based mRNA delivery, the C14-4 LNPs caused less toxicity in the treated T cells and resulted in similar amounts of surface CAR expression in transfected T cells. In a co-culture assay with ALL cells, LNP-generated CAR T cells demonstrated the same potent cancer cell killing ability as EP-generated CAR T cells. Thus, LNPs were validated as an alternative strategy for mRNA-based ex vivo engineering of CAR T cells.

Based off of this data, C14-4 LNPs can provide a novel method for T cell engineering in both clinical manufacturing settings, such as for generating CAR T cells and research settings. Because C14-4 was able to enhance mRNA delivery when compared to lipofectamine (a gold-standard transfection reagent in cell-based research), it can be used to study T cells beyond CAR applications. The subsequent investigations have shown promising results for optimizing the C14-4 LNP formulation to further enhance mRNA delivery to T cells and T cell lines. C14-4 LNPs were also shown to deliver CD-19 CAR mRNA to primary human T cells as effectively as EP with lower toxicity.

### Characterization of LNP Library

In this study, ionizable lipid nanoparticles (LNPs) were investigated for mRNA delivery to T cells. LNPs were selected because they have been shown to deliver mRNA intracellularly with high potency and low toxicity to a range of cell and tissue targets in vivo and ex vivo. Most recently, LNPs have been utilized for nucleic acid delivery to a range of immune cell types (Berdeja J G et al., 2017, J Clin Oncol, 35; Oberli M A et al., 2017, Nano Lett, 17:1326-1335; Love K T et al., 2010, Proc Natl Acad Sci, 107:9915-9915; Kauffman K J et al., 2015, Nano Lett, 15:7300-7306; Midoux P et al., 2014, Expert Rev Vaccines, 14:221-234; Lokugamage M P et al., 2019, Adv Mater, 1902251:1-8).

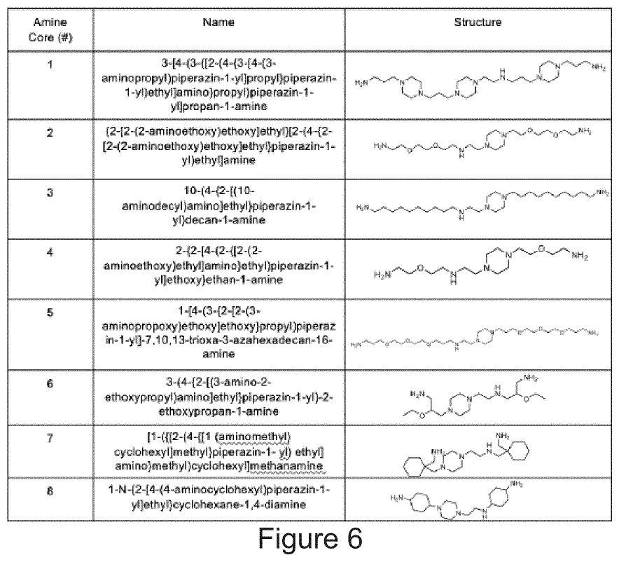

To investigate mRNA delivery specifically to T cells, a library of 24 different LNP formulations was generated by first synthesizing ionizable lipid materials via Michael addition chemistry, where polyamine cores were reacted with an excess of epoxide-terminated alkyl chains of varying lengths (FIG. 2 and FIG. 6). The specific ionizable lipids synthesized in this library are structural analogs of an ionizable lipid that was previously formulated into LNPs and shown to deliver siRNA and mRNA to immune cells (Oberli M A et al., 2017, Nano Lett, 17:1326-1335; Love K T et al., 2010, Proc Natl Acad Sci, 107:9915-9915; Leuschner F et al., 2012, Nat Biotechnol, 29:1005-1010).

The lipids were evaluated for mRNA delivery to T cells specifically rather than a range of cell types. To formulate LNPs, ionizable lipids were combined in ethanol with three other excipients: (i) cholesterol for LNP stability and membrane fusion, (ii) DOPE to fortify the bilayer structure of the LNP and promote endosomal escape, and (iii) C14-PEG to reduce aggregation and nonspecific endocytosis (Granot Y et al., 2017, Semin Immunol, 34:68-77; Varkouhi A K et al., 2011, J Control Release, 151:220-228; Mui B L et al., 2013, Mol Ther Acids, 2:1-8). This ethanol phase was then mixed with aqueous phase mRNA in a microfluidic device (FIG. 1A). These excipients and their molar ratios were chosen based off of previously optimized LNP formulations for mRNA delivery, which generally utilized (i) DOPE as the phospholipid component, (ii) a decreased molar percentage of ionizable lipid, and (iii) increased concentrations of cholesterol and lipid-PEG (Kauffman K J et al., 2015, Nano Lett, 15:7300-7306; Ball R L et al., 2018. Nano Lett, 18:3814-3822). Given that alterations in the molar ratio of excipients impact the physicochemical properties and ultimately potent delivery of LNPs, the ratio of the components was held constant throughout these experiments (Kauffman K J et al., 2015, Nano Lett, 15:7300-7306; Ball R L et al., 2018, Nano Lett, 18:3814-3822; Cheng Q et al., 2018, Adv Mater, 30:1805308).

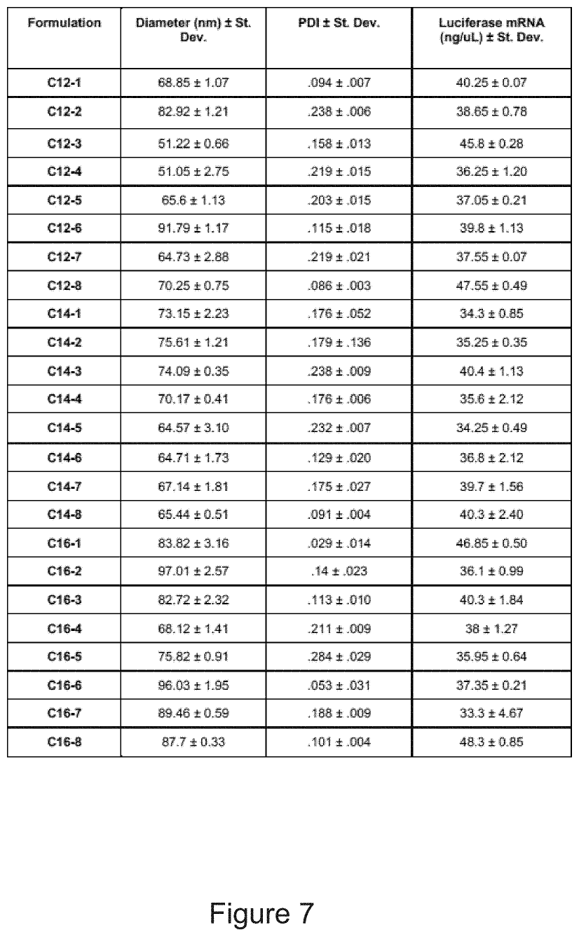

The resulting LNPs were then characterized for size and mRNA concentration using dynamic light scattering (DLS) and A260 absorbance measurements. The diameter of the LNPs, reported as the z-average measurement, ranged from 51.05 to 97.01 nm with PDIs below 0.3 (FIG. 7). The mRNA concentration measured as A260 absorbance showed consistency across LNP formulations, ranging from 33.3 to 48.3 ng/μL. Collectively, these results confirmed the formulation of 24 different LNP formulations encapsulating mRNA to be used in this investigation for T cell delivery.

Screening of LNPs for mRNA Delivery to Jurkat Cells

To evaluate the LNPs for their ability to deliver functional mRNA, luciferase was chosen as the encoded reporter protein. After the addition of luciferin, only luciferase protein translated from the mRNA reacts to generate luminescent signal, creating an easily detectible output that correlates with functional mRNA delivery (Hajj K A et al., 2019, Small, 15:1-7). The luciferase mRNA used in these experiments utilized N1-Methyl-PseudoU and 5-Methyl-C modifications, which have been shown to enhance mRNA translation and successfully encapsulate within LNPs (Pardi N et al., 2015, J Control Release, 217:345-351; Svitkin Y V et al., 2017, Nucelic Acids Res, 45:6023-6036; Trixl L et al., 2018, WIREs RNA, 10:1-17). These modifications may alter mRNA encapsulation in LNPs, delivery of the mRNA, and overall immunogenicity, so further investigation into the optimized modifications for these specific LNP delivery vehicles can be explored in future work (Pardi N et al., 2015, J Control Release, 217:345-351; Zhang R et al., 2018, J Control Release, 292:256-276; Karikó K et al., 2005, Immunity, 23:165-175; Li J et al., 2017, ACS Nano, 11:2531-2544; Shen X et al., 2018, Nucleic Acids Res, 46:1584-1600; Sahin U et al., 2014, Nat Rev Drug Discovery, 13:759-780).

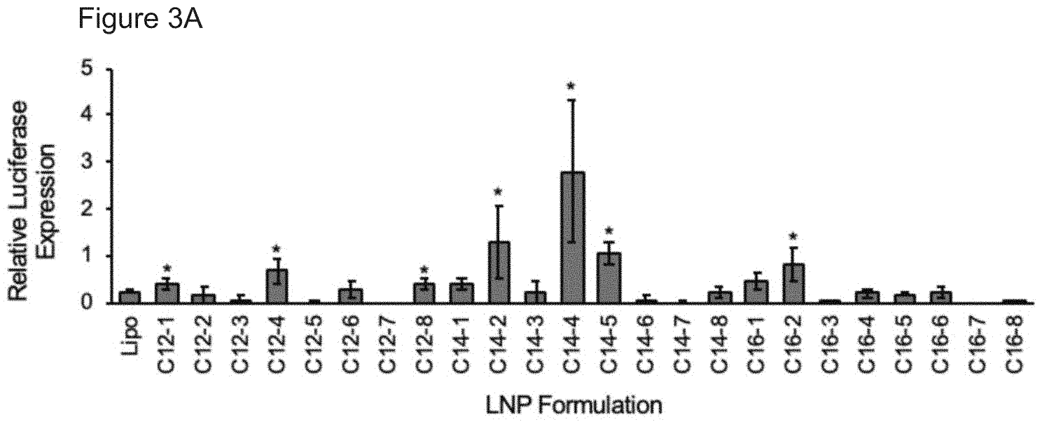

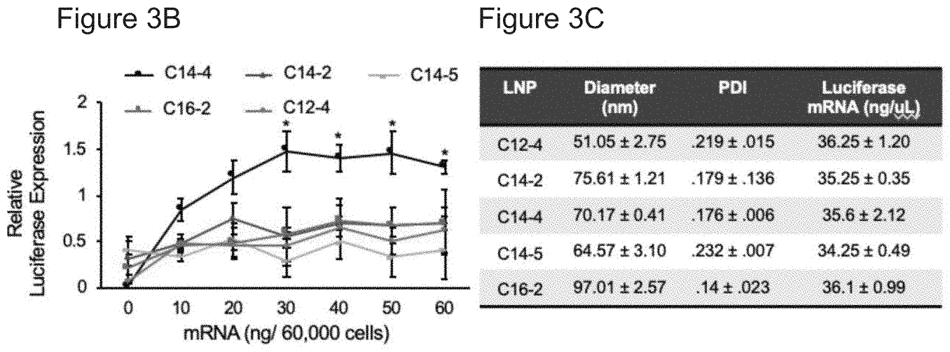

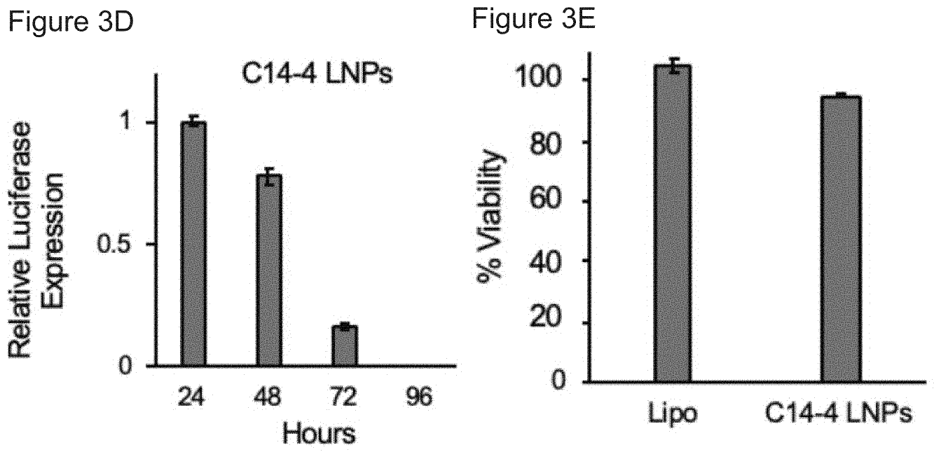

Functional delivery of luciferase mRNA was observed using Jurkat cells, a line of immortalized human T cells commonly utilized to study T cell behavior (Olden B R et al., 2018, J Control Release, 282:140-147; Abraham R T et al., 2004, Nat Rev Immunol, 4:1-8; Cancer P et al., 2018, Nucleic Acid Ther, 28:285-296). LNPs encapsulating luciferase mRNA were used to treat Jurkat cells at a concentration of 30 ng/60,000 cells. After 48 hrs, luciferase expression was assessed via luminescence measurements. The luminescent measurements from LNP formulations were normalized to an untreated cell group and compared to commercially available lipofectamine, a commonly used transfection reagent widely considered the gold standard in vitro (Cardarelli F et al., 2016, Sci Reports, 1-8; Wang T et al., 2018, Molecules, 23). The library screen revealed seven LNP formulations that resulted in significantly higher luciferase expression than lipofectamine, indicating an improved ability to deliver luciferase mRNA to Jurkat cells (FIG. 3A). Of these seven, three formulations had ionizable lipids with C12 tails, three had C14 tails, and one had C16 tails. Polyamine cores 3, 6, and 7 did not enhance transfection compared to lipofectamine, regardless of the lipid tail length. However, polyamine cores 2, 4, and 5, all with similar structures of only one ring and additional oxygens, were responsible for producing the five formulations with the highest resulting luciferase expression, i.e., C14-4, C14-2, C14-5, C16-2, and C12-4 LNPs.

These top five LNP formulations were then compared over a range of mRNA concentrations to determine both the top LNP formulation and the optimal LNP dose for Jurkat cell transfection. The results confirmed that C14-4 LNPs, the top performing LNP formulation from the original library screen, induced the highest luciferase expression out of the top five formulations (FIG. 3B). The increase in luciferase expression was significant compared to all other LNP formulations at doses greater than 20 ng, indicating that the optimal dose for C14-4 LNPs in Jurkat cells was 30 ng. The enhanced performance of C14-4 LNPs does not reflect a difference in size or mRNA concentration, as the formulation has a diameter of 70.17 nm and a concentration of 35.6 ng/μL (FIG. 3C). The toxicity of C14-4 LNPs on Jurkat cells was minimal and cell viability was comparable to lipofectaminetreated and untreated cell groups with greater than 95% viability measured after treatment with C14-4 LNPs.

Further, to verify transient expression of mRNA delivered via C14-4, luciferase expression in Jurkat cells treated with LNPs was observed over 96 hrs. The results showed a 23% decrease in expression at 48 hrs compared to 24 hrs and an 84% decrease by 72 hrs, with no detectable expression by 96 hrs (FIG. 3D), confirming transient luciferase expression and informing the use of 24 hrs timepoint for subsequent experiments. Collectively, these results allowed for the selection of C14-4 LNPs as the top formulation for mRNA delivery and provided the optimized transfection methods for C14-4 LNPs in vitro.

Lipid Nanoparticle-Mediated mRNA Delivery to Primary Human T Cells

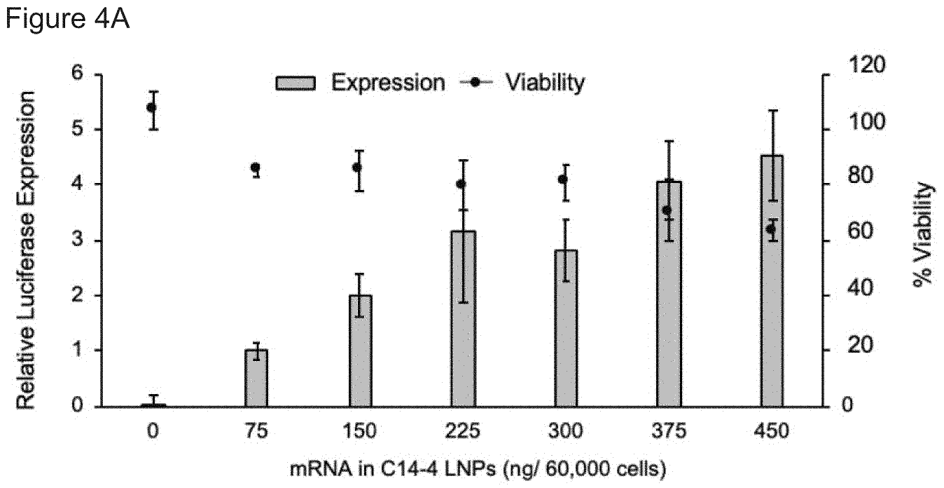

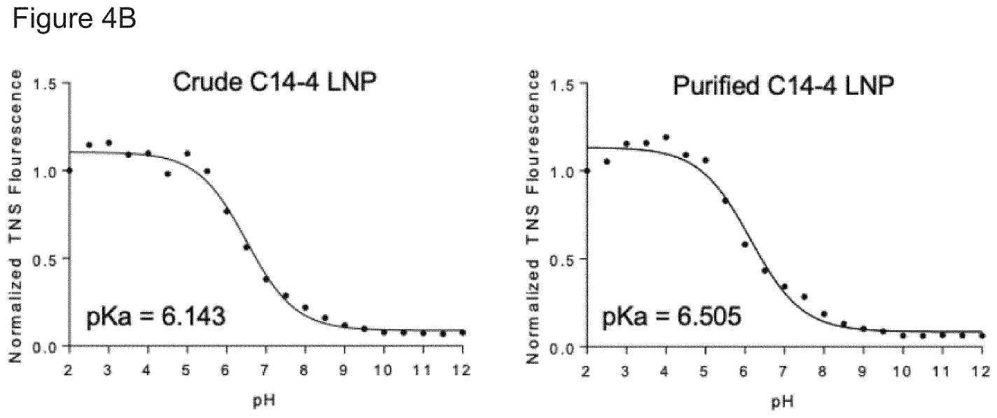

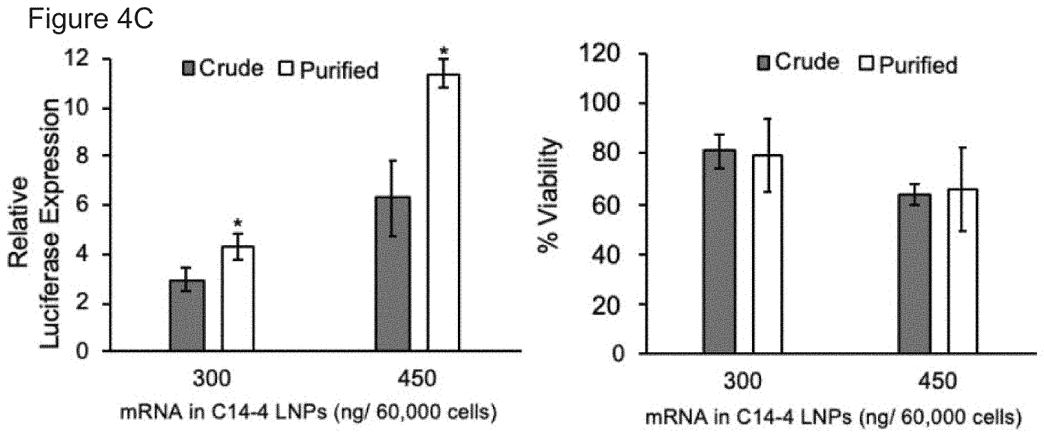

Because CAR T cells used for cancer immunotherapy in the clinic are generated using harvested patient T cells (CD3+), the top-performing C14-4 LNPs were utilized for mRNA delivery to primary human T cells to demonstrate translatability beyond the Jurkat cell line.V Limitations of the Jurkat cell line include that is derived from only CD4+ T cells whereas primary T cells also include CD8+ phenotypes (Abraham R T et al., 2004, Nat Rev Immunol, 4:1-8). However, primary T cells require activation to achieve transfection (Barrett D M et al., 2011. Hum Gene Ther, 22:1575-1586; Harrer D C et al., 2017, BMC Cancer, 17:551). Dynabeads, widely and clinically utilized magnetic beads with a surface coated in CD3 and CD28 antibodies, were utilized for the activation of T cells in a similar fashion as those used in CAR T clinical trials (Hajj K A et al., 2019, Small, 15:1-7; Wang X et al., 2016, Mol Ther Oncolytics, 3:1-7; Lee D W et al., 2015, Lancet, 385:517-528). The isolated T cells were suspended at a 1:1 ratio of CD4+:CD8+ and treated with C14-4 LNPs encapsulating luciferase mRNA at a range of concentrations. After 24 hrs, luciferase expression and cell viability were quantified (FIG. 4A). The LNPs induced luciferase expression in T cells in an mRNA dose-dependent manner, indicating successful delivery of luciferase mRNA to the T cells. Further, minimal toxicity was observed at only the highest doses, indicating the biocompatibility of C14-4 LNPs with primary cells.

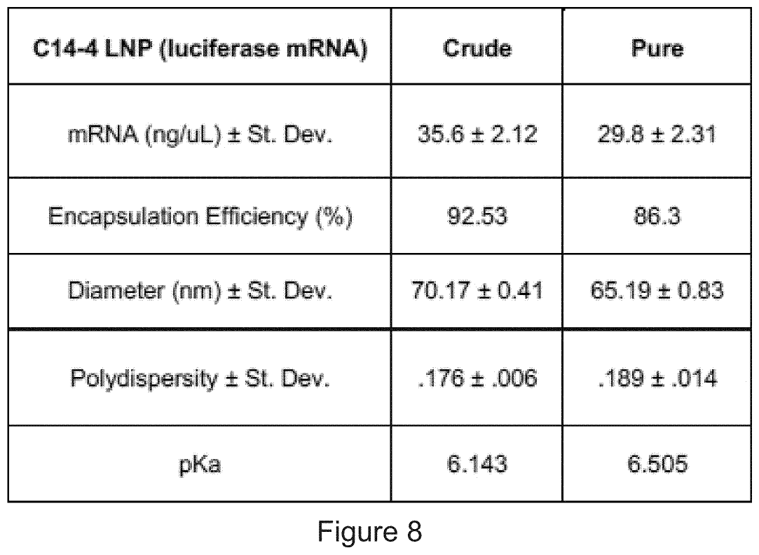

To further explore the potential of C14-4 for mRNA delivery to T cells, the fully saturated ionizable lipid was purified via flash chromatography, and the purified product was utilized to produce C14-4 LNPs. These purified C14-4 LNPs were compared with C14-4 LNPs made from crude C14-4 ionizable lipids to verify which structure was responsible for potent mRNA delivery. DLS and A260 absorbance characterization of the purified C14-4 LNP revealed a diameter of 65.19 nm and mRNA concentration of 29.8 ng/μL, which did not greatly differ from the LNPs made with crude C14-4 product (FIG. 8). Using a Ribogreen assay to evaluate the ability of each formulation to encapsulate mRNA, it was revealed that the crude and purified formulations had similar encapsulation efficiencies of 92.5% and 86.3%, respectively. Lastly, in a TNS assay, the two LNP formulations were evaluated for their surface ionization, or pKa, which is defined as the pH at which the LNPs are 50% protonated and is indicative of how pH affects the surface charge and stability of the LNP (Hajj K A et al., 2019, Small, 15:1-7). Ionizable lipids have a pKa below 7, which allows them to become charged in acidic endosomal compartments, resulting in the release of encapsulated mRNA (Hajj K A et al., 2019, Small, 15:1-7; Zhang J et al., 2011, Langmuir, 27:9473-9483). Both the crude and purified C14-4 LNPs were shown to be ionizable, with the purified formulation having a slightly higher pKa value (FIG. 4B).

The crude and purified C14-4 LNPs were then compared for their ability to deliver mRNA in primary T cells. The T cells were suspended at a 1:1 ratio of CD4+ to CD8+ and activated with Dynabeads before treatment with LNPs. Crude and purified C14-4 LNPs encapsulating luciferase mRNA were investigated at two concentrations for luciferase expression and viability (FIG. 4C). At both concentrations, the purified C14-4 LNPs had significantly increased luciferase expression compared to the crude LNP formulation, and both formulations had minimal effects on cell viability. Overall, the increase in luciferase expression without any increase in toxicity indicates purified C14-4 LNPs as the top-performing formulation for primary T cell mRNA delivery.

LNPs Deliver CAR mRNA as Effectively as EP to Primary T Cells with Lower Toxicity

After quantifying functional luciferase-encoded mRNA delivery to T cells via luminescence measurements, C14-4 LNPs were utilized for CAR mRNA delivery as a clinically relevant application of the delivery vehicle. CAR T cells generated with mRNA have been utilized in numerous clinical trials, as initial investigations suggested that transient CAR expression may overcome obstacles associated with toxicity and off-target effects (Barrett D M et al., 2011, Hum Gene Ther, 22:1575-1586; Zhao Y et al., 2010, Cancer Res, 70:9053-9061; Foster J B et al., 2019, Mol Ther, 27:747-756). However, in these investigations, the mRNA was delivered to T cells via electroporation (EP), a commonly used, potent but toxic method of transfection that relies on electrically permeabilizing T cell membranes (Smits E et al., 2004, Leukemia, 18:1898-1902; Barrett D M et al., 2011, Hum Gene Ther, 22:1575-1586; Svoboda J et al., 2018, Blood, 132:1022-1027; DiTommaso T et al., 2018, PNAS, 115; Dullaers M et al., 2004, Mol Ther, 10:768-779; Singh N et al., 2014, Cancer Immunol Res., 2:1059-1070). Here, C14-4 LNPs encapsulating CD19+ CAR mRNA were compared to EP to determine their ability to generate functional CAR T cells without causing toxicity.

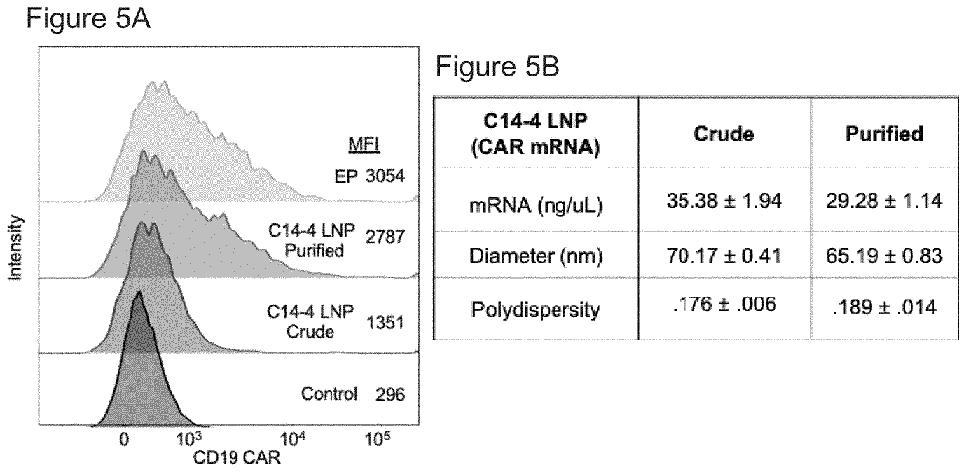

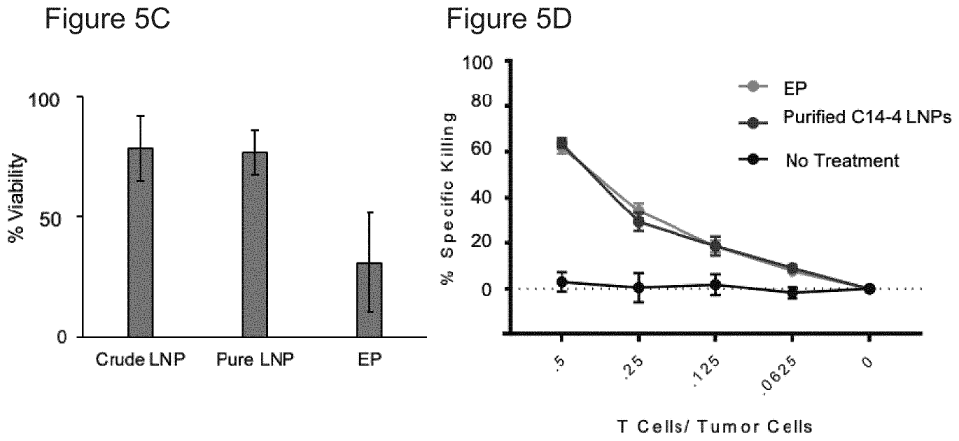

LNPs were used to treat primary T cells at a 1:1 CD4+:CD8+ ratio in comparison to EP at identical 450 ng/μL mRNA concentrations. The resulting T cell populations were analyzed for CD19+ CAR expression using fluorophore labeled antibodies and flow cytometry, and they were quantified in terms of mean fluorescence intensity (MFI) (FIG. 5A). The highest resulting MFIs came from the T cells treated using EP and purified C14-4 LNPs, while crude C14-4 LNPs generated a more modest MFI. When these values were normalized to the untreated control group of T cells, EP showed a 10-fold increase in MFI over untreated cells and purified C14-4 LNPs showed a 9.4-fold increase while crude C14-4 LNPs only increased MFI by 4.5-fold. Thus, as observed with luciferase mRNA delivery, the crude C14-4 formulation was less effective in inducing expression than its purified counterpart. The viability of T cells treated with LNP formulations in comparison to EP treatment was also quantified (FIG. 5C), indicating a significantly reduced toxicity with LNP treatment compared to EP. As seen previously with luciferase mRNA delivery, the purified and crude LNPs have a similarly high viability of 76% and 78%, respectively. This was contrasted by the 31% viability observed in EP treated T cells, emphasizing the cytotoxic nature of this currently utilized mRNA delivery process. Overall, these results highlight the safety benefits of utilizing C14-4 LNPs over EP for mRNA delivery to T cells ex vivo.

With EP and purified C14-4 LNPs inducing similar CD19+ CAR expression in T cells, the two mRNA delivery methods were evaluated for functional CAR expression using a co-plated cancer cell killing assay. By plating the engineered CAR T cells with CD19+ acute lymphoblastic leukemia (ALL) cells that express luciferase, Nalm6, cancer cell killing can be assessed as decrease in luminescence as compared to Nalm6 cells alone. Both this assay and cell line have been utilized routinely in previous studies to demonstrate CAR T cell functionality and therapeutic potential (Barrett D M et al., 2011, Hum Gene Ther, 22:1575-1586; Castella M et al., 2019, Mol Ther Methods Clin Dev, 12:134-144; An N et al., 2016, Oncotarget., 7; Li X et al., 2018, Cell Rep, 23:2130-2141; Pan Y et al., 2018, PNAS, 115:1-6). Here, CAR T cells engineered with mRNA delivered via purified C14-4 LNPs or EP were compared at a range of T cell to effector cell ratios. After 48 hrs, the EP and C14-4 LNP treatment groups performed nearly identically and resulted in more cancer cell killing than the untreated group (FIG. 5A). Thus, the purified C14-4 LNPs successfully generated mRNA-induced CAR T cells comparable to EP, the current standard of delivery for mRNA CAR T cells. Collectively, these results validate purified C14-4 LNPs as a method for mRNA-based CAR T cell engineering with similar potency and reduced T cell toxicity compared to EP.

In summary, the data described herein disclose a novel ionizable lipid and novel LNP formulations that are effective for mRNA delivery to T cells. The present invention, in part, addresses the problem of targeted delivery of mRNA to T cells using a novel LNP system. The present invention discloses, in part, an ionizable lipid referred to as C14-4 and its LNP formulation (including cholesterol, phospholipid, and PEG components) that has been utilized for the potent delivery of mRNA to T cells. Both the crude lipid and purified fully saturated lipid were utilized. The studies described herein also demonstrate the ability of C14-4 and the C14-4 LNP formulation to deliver mRNA to T cells with low toxicity and enhanced efficacy of the current gold-standard reagent lipofectamine gives C14-4 the potential to change the way T cells are engineered. This can apply to clinical areas, such as CAR T cell therapeutics, where the invention has future commercial potential, but it can also apply to lab-based/research settings as T cells are notably hard to transfect.

Additional studies move to use this lipid nanoparticle for in vivo mRNA delivery to T cells, possibly with the addition of antibody-based targeting agents or other targeting ligands. Further optimization of the LNP formulation in terms of excipient ratios (varying the molar ratios of phospholipid, cholesterol, PEG, and C14-4) and the ratio of ionizable lipid to mRNA is also investigated. This may lead to the introduction of new excipients or modifications to the C14-4 lipid itself, such as using branched alkyl chains instead of linear. Further, other mRNA cargo beyond luciferase and CAR is explored.

The materials and methods employed in these experiments are now described.

Lipid Synthesis

Ionizable lipids were synthesized by reacting epoxide terminated alkyl chains (Avanti Polar Lipids) with polyamine cores (Enamine, Monmouth Jct, N.J.) using Michael addition chemistry. The components were combined with a 7-fold excess of alkyl chains and mixed with a magnetic stir bar for 48 hrs at 80° C. The crude product was then transferred to a Rotavapor R-300 (BUCHI, Newark, Del.) for solvent evaporation, and the lipids were suspended in ethanol. Finally, to purify the top-performing lipid (C14-4), the lipid fractions were separated via CombiFlash Nextgen 300+ chromatography system (Teledyne ISCO, Lincoln, Nebr.) and the saturated lipid fraction was identified by molecular weight using liquid chromatography-mass spectrometry.

CAR mRNA Synthesis

mRNA was produced using standard in vitro transcription methods, as previously described (Singh N et al., 2014, Cancer Immunol Res, 2:1059-1070). Briefly, plasmid DNA encoding a second-generation lentiviral vector targeting CD19 and bearing the CD3zeta and 4-1BB costimulatory domains was linearized overnight, followed by production of mRNA using the T7 mMessage ULTRA kit (Thermo Fisher) as per manufacturer instructions. mRNA was then polyA tailed and capped, and purified using the RNeasy mini kit (Qiagen).

LNP Formulation and Characterization

To synthesize LNPs, an aqueous phase containing mRNA and ethanol phase containing lipid and cholesterol components were mixed using a microfluidic device as previously described (Chen D et all., 2012, J Am Chem Soc, 134:6948-6951). Briefly, the aqueous phase was prepared using 10 mM citrate buffer and either luciferase mRNA with N1-Methyl-PseudoU and 5-Methyl-C substitutions (Trilink Biotechnologies, San Diego, Calif.) or CAR mRNA (synthesized as described above) at 1 mg/mL. To prepare the ethanol phase, ionizable lipid, 1,2-distearoyl-sn-glycero-3-phosphoe-thanolamine (DOPE) (Avanti Polar Lipids, Alabaster, Ala.), cholesterol (Sigma, St. Louis, Mo.), and lipid-anchored PEG (Avanti Polar Lipids) components were combined at a set molar ratio of 35%, 16%, 46.5%, and 2.5%, respectively. Pump33DS syringe pumps (Harvard Apparatus, Holliston, Mass.) were used to mix the ethanol and aqueous phases at a 3:1 ratio in a microfluidic device (Chen D et all., 2012, J Am Chem Soc, 134:6948-6951). After mixing, LNPs were dialyzed against 1×PBS for 2 hrs before sterilization via 0.22 μm filters. Dynamic light scattering (DLS) performed on a Zetasizer Nano (Malvern nstruments, Malvern, UK) was then used to measure, in triplicate, the diameter (z-average) and polydispersity index (PDI) of the LNPs suspended in Ix PBS. A NanoDrop ND-1000 Spectrophotometer (ThermoFisher, Waltham, Mass.) was used to obtain the mRNA concentration of each LNP formulation.

Further analysis of top-performing LNP formulations included Quant-iT Ribogreen (ThermoFisher) and 6-(ptoluidinyl) naphthalene-2-sulphonic acid (TNS) assays to determine the encapsulation efficiency and pKa of the LNPs, respectively. The Quant-iT Ribogreen was performed as previously described (Heyes J et al., 2005. J Control Release, 107:276-287). Briefly, equal concentrations of LNPs were treated with Triton X-100 (Sigma) to lyse the LNPs or left untreated, and after 10 min, the groups were plated in triplicate in 96 well-plates alongside RNA standards. The fluorescent Ribogreen reagent was added per manufacturer instructions, and the resulting fluorescence was measured on a plate reader. The values were compared to the standard curve to quantify RNA content, and encapsulation efficiency was calculated. To determine LNP pKa, a TNS assay was used to measure surface ionization as previously described (Hajj K A et al., 2019, Small, 15:1-7.). Buffered solutions of 150 mM sodium chloride, 20 mM sodium phosphate, 25 mM ammonium citrate, and 20 mM ammonium acetate were adjusted to reach pH values ranging from 2 to 12 in increments of 0.5. LNPs were added to each pH-adjusted solution in triplicate wells in a 96 well-plate. TNS was then added to each well to reach a final TNS concentration of 6 JIM, and the resulting fluorescence was read on a plate reader. The pKa was then calculated as the pH at which the fluorescence intensity was 50% of its maximum value—reflective of 50% protonation.

mRNA Transfection of Jurkat Cells

Jurkat cells (ATCC TIB-152), an immortalized human T cell line (Abraham R T et al., 2004, Nat Rev Immunol, 4:1-8), were cultured in RPMI-1640 with L-glutamine (ThermoFisher,) supplemented with 10% fetal bovine serum and 1% penicillin-streptomycin. Cells were plated at 60,000 cells per well in 96-well plates in 60 μL of media and were immediately treated with 60 μL of LNPs diluted in PBS to varying concentrations. Lipofectamine MessengerMAX transfection reagent (ThermoFisher), used ss a positive control comparison, was combined with mRNA for 10 min per the manufacturer protocol and used to treat wells using the same mRNA concentration as the LNP groups. After 48 hrs of incubation, the cells were centrifuged at 300×g for 4 min and resuspended in 50 μL of 1× lysis buffer (Promega, Madison, Wis.) and 100 μL of luciferase assay substrate (Promega). The luminescence was then quantified using an Infinite M Plex plate reader (Tecan, Morrisville, N.C.). The luminescent signal from each group was normalized to either untreated cells or the lowest concentration treatment group, and background, measured as wells with reagents but no cells, was subtracted. To assess cytotoxicity, Jurkat cells were plated under the same conditions and treated with either C14-4 or lipofectamine at 30 ng mRNA per 60.000 cells. After 48 hrs, 60 μL of CellTiter-Glo (Promega) was added to each well, and luminescence corresponding to ATP production was quantified using a plate reader. The luminescent signal from each group was normalized to untreated cells, and background was subtracted.

mRNA Transfection of Primary T Cells

Primary T cells (CD3+) were obtained from the University of Pennsylvania Human Immunology core and combined at a 1:1 ratio of CD4:CD8. Cells to be treated with LNPs were then activated overnight with Human Tactivator CD3/CD28 Dynabeads (ThermoFisher) at a 3:1 bead to cell ratio. After activation, the cells were plated at 60,000 cells per well in 96-well plates in 60 μL of media and treated with LNPs at varying mRNA concentrations. For electroporation, T cells were washed three times with media, resuspended 108 cells/mL, and mixed with transcribed mRNA at a concentration of 100 pg mRNA per 1 mL T cells. The cells were then electroporated in a 2-mm cuvette using an ECM830 Electro Square Wave Porator (Harvard Apparatus BTX). For experiments with luciferase mRNA treatments, the same protocols described above were used to assess luminescence after 48 hrs and toxicity after 24 hrs. For CAR mRNA treatments, surface CAR expression was detected using an antiidiotype antibody to the CD19 CAR, generously provided by Novartis Pharmaceuticals. Toxicity of the CAR mRNA treatments was assessed as described above, using a CellTiter Glo kit.

Functional Assays

CAR T cells were co-plated with luciferase-expressing Nalm-6 cells, an ALL cell line, at varying effector-to-target ratios. After 48 hrs in co-culture, D-luciferin potassium salt (Perkin-Elmer, Waltham, Mass.) was added to cell cultures to reach a final concentration of 15 pg/mL and incubated at 37° C. for 10 min. Luminescence was then detected using a Synergy H4 imager (BioTek, Winooski, Vt.), and signal was analyzed using BioTek Gen5 software. Percent specific lysis was calculated using a control of target cells without effectors.

### Example 2: Engineering Lipid Nanoparticles for T Cell Delivery

Further optimization of the C14-4 formulation parameters in terms of the excipient ratios has already began. Attached are the two libraries of formulations that were generated (Library A and subsequently Library B, based on the results from Library A; representative formulations from Library A are named A#, and those from Library B are B#). Both were made using the C14-4 lipid, but some of the new formulations demonstrated enhanced mRNA delivery in a T cell line over the original C14-4 formulation without increasing toxicity.

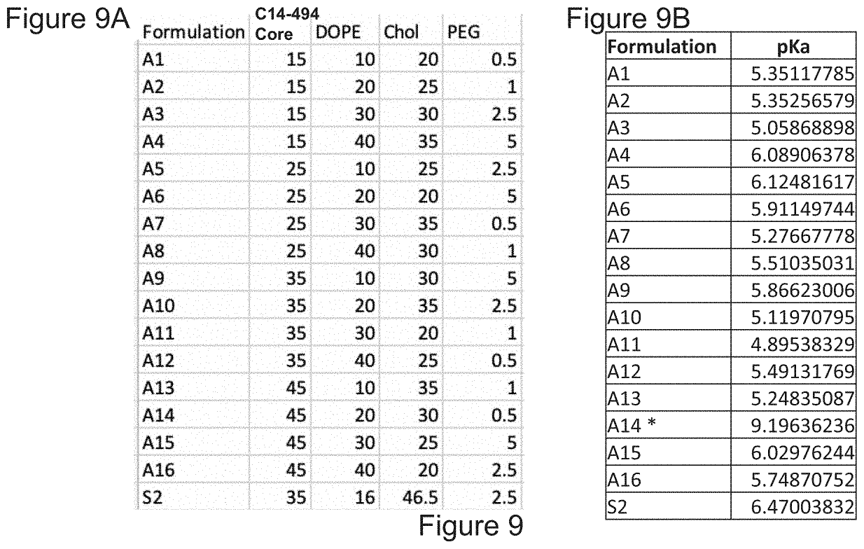

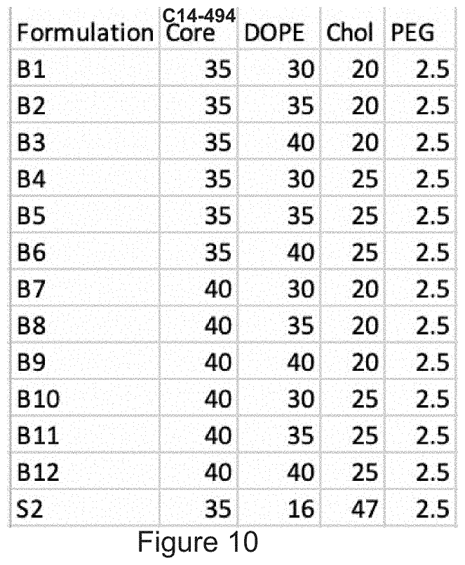

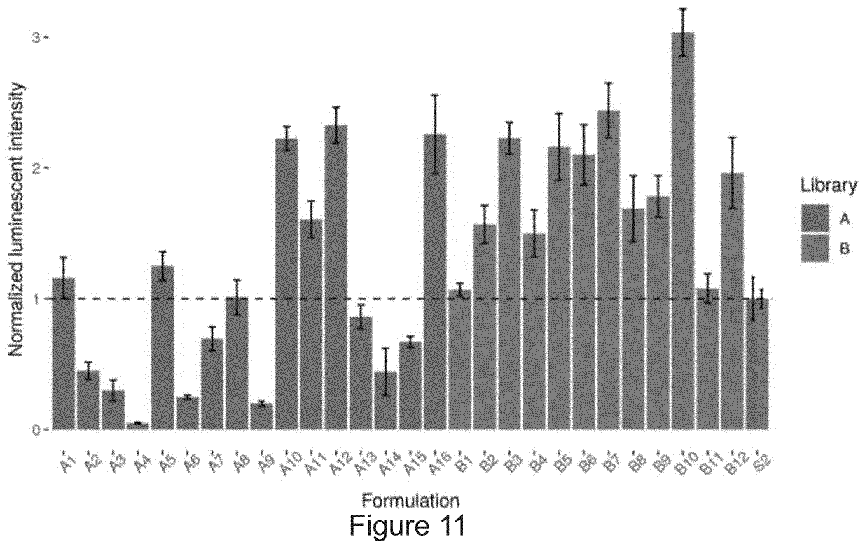

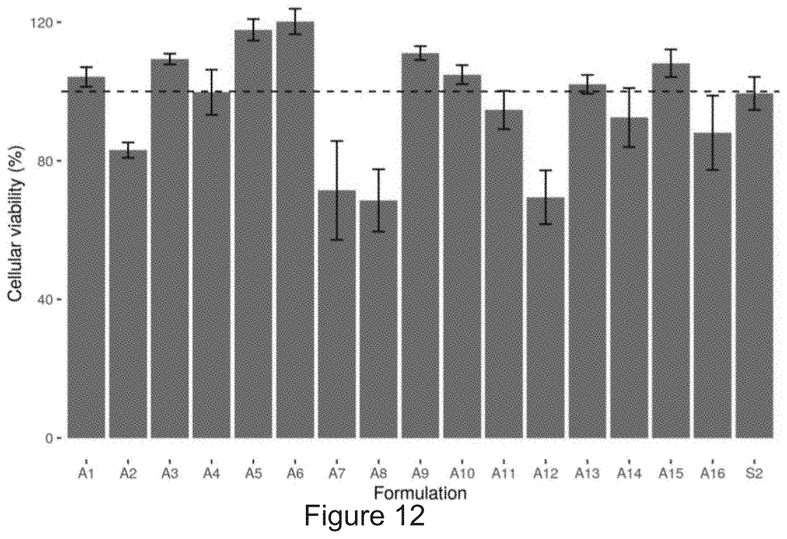

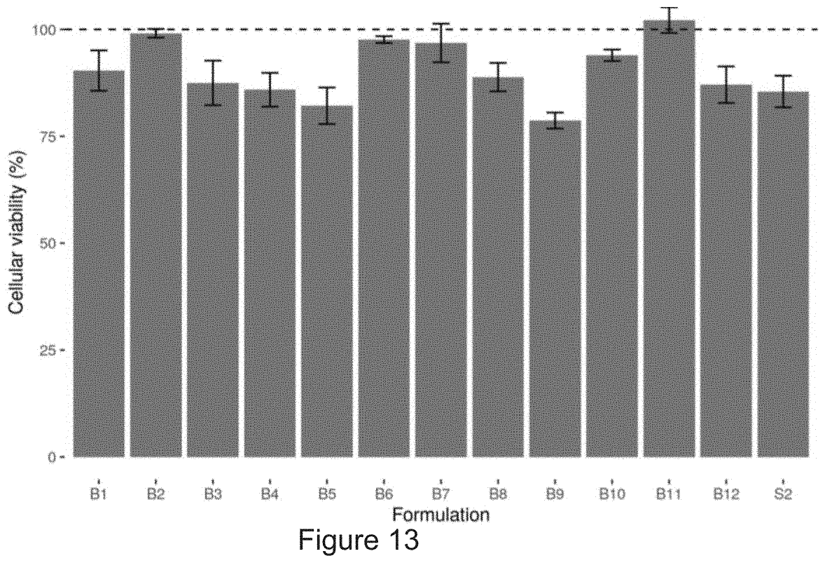

Ionizable LNPs showed great promise as vehicles for the intracellular delivery of therapeutic macromolecules, including nucleic acids (Mukalel A. J., 2019, Cancer Lett, 458:102-112). LNP formulations are numerous, but they utilize common excipients: cholesterol for membrane stability, phospholipid to assist with endosomal escape, and polyethylene glycol (PEG) to reduce immunogenicity (Reichmuth A. M., 2016, Ther Deliv, 7:319-334). Varying excipient combinations can significantly change the physicochemical properties of LNPs, thereby influencing their delivery capabilities (Kauffman K., 2015, Nano Lett, 15:7300-7306). In the course of this study, two libraries of LNPs were engineered for T cells (FIG. 9 and FIG. 10). The formulations were chosen using orthogonal DOE design so that a large range of component variation was able to be observed with only sixteen representative formulations.

Each formulation contained varying molar ratios of ionizable lipid, cholesterol, helper lipids, and lipid-conjugated PEG. The z-average diameter and pKa of each formulation was determined using dynamic light scattering, 2-(p-toluidinyl) naphthalene-6-sulfonic acid (TNS) assays, and 260 nm absorbance measurements, respectively. Jurkat cells, immortalized human T cells, were treated with each formulation for 48 hrs and assess for in vitro intracellular delivery. The cytotoxicity of each formulation was similarly assessed through the commercial Cell-Titer Glo assay.

Data on optimized formulations are shown in FIG. 9 and FIG. 10. The in vitro delivery efficiency of each formulation was assessed using a standard luciferase expression assay. Briefly, LNPs containing mRNA encoding firefly luciferase were delivered to Jurkat cells, immortalized human T cells, at an mRNA concentration of 30 ng per 60,000 cells. After 48 hrs of incubation, the cells were lysed and treated with firefly luciferin. The extent of LNP-mediated transfection was then measured as luminescence intensity on a plate reader. The cytotoxicity of each formulation was similarly assessed using the commercial Cell-Titer Glo assay.

Characterization of Library A revealed a number of trends in delivery tied to excipient composition. The optimal excipient conditions in library A led to the development of a next-generation library B. The formulations in Library B demonstrated much greater encapsulation efficiency and a larger z-average diameter than those in Library A and a number of formulations in Library B outperformed even the top-performing formulations from Library A, supporting the abovementioned trends. Additionally, all formulations in Library B were observed to exhibit over 80% viability over the course of 48 hrs. Thus, the development of multiple highly potent LNP formulations for intracellular delivery to T cells is reported. These LNPs have potential for use in future T cell engineering applications, including cancer immunotherapy.

### Example 3: Altering the Excipient Composition of LNPs to Improve their Ability to Deliver mRNA to T Cells (with Minimal Toxicity)

The present example demonstrates the in vitro and ex vitro data obtained for representative Library A and Library B formulations. In in vitro studies, Library A, comprising sixteen representative formulations of C14-494 with varied excipient concentrations (e.g., FIG. 9A), was screened for ability to deliver luciferase mRNA to Jurkat cell line (immortalized human T cells). Library B (e.g., FIG. 10), generated based off of Library A results, was also screened in Jurkats in in vitro studies. Further ex vivo studies focused on the delivery of luciferase mRNA to primary T cells using representative top performing formulations of Library A and B.

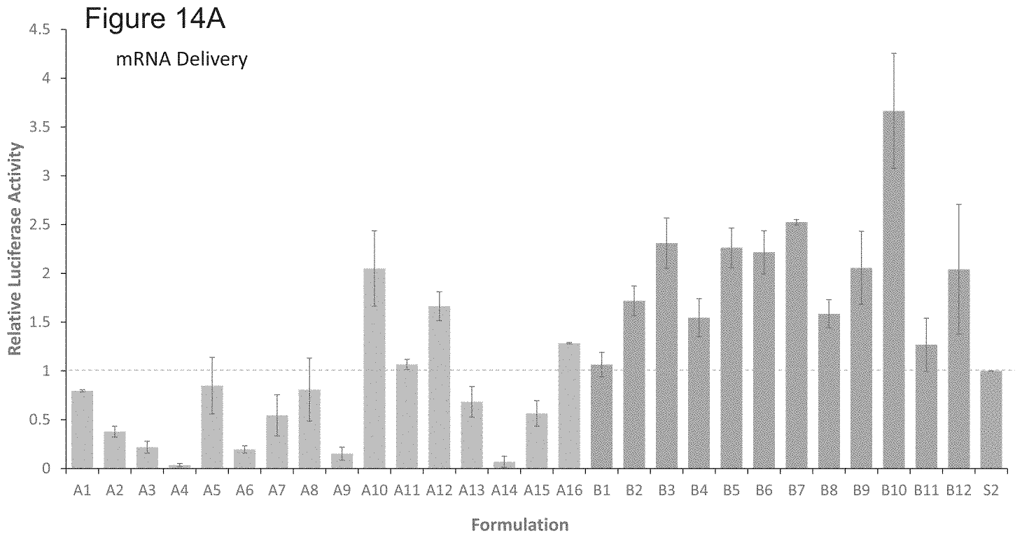

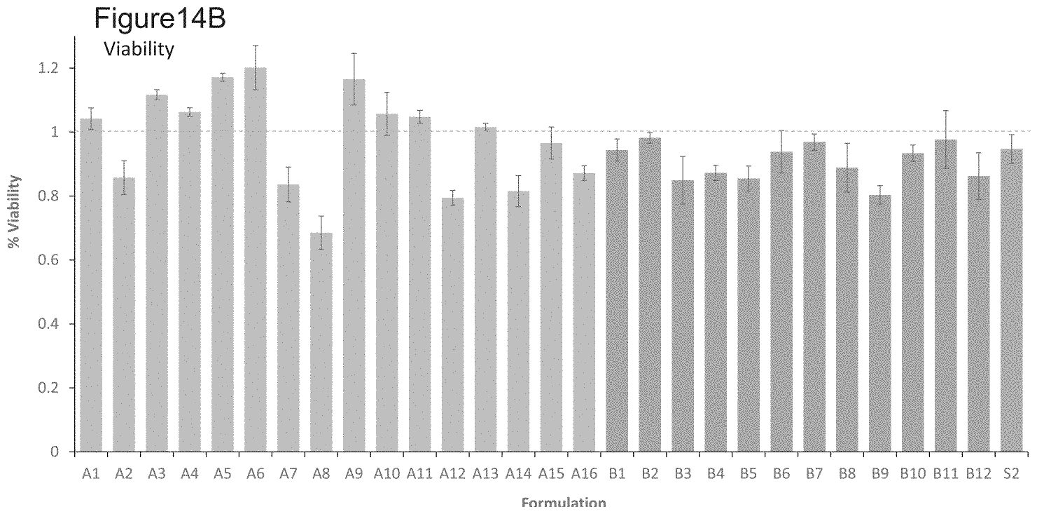

More specifically, Jurkats were treated for 24 hr with 30 ng/60,0000 cells. The adjustments made to Library B based on the data from Library A led to more “hit” formulations (aka those that achieved higher delivery than the standard formulation S2) and led to less toxicity overall in LNP formulations. Luciferase activity was measured 24 hr after incubation with LNPs (containing luciferase mRNA) using luciferase assay (FIG. 14A). Percent viability was measured at same timepoint with Cell Titer Glo assay (FIG. 14B). Each bar contains three biological replicates (with three technical replicates each) and were normalized to 0 ng treatment.

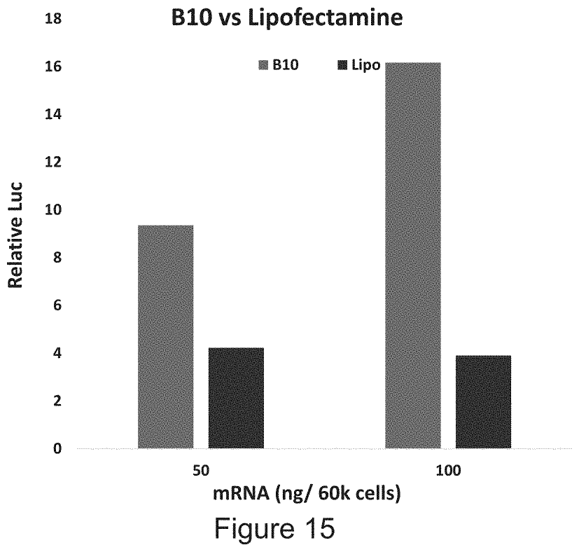

The Lipofectamine comparison (FIG. 15) showed that formulation B10 out-performed this commercially available standard. Furthermore, toxicology results demonstrated that neither were toxic to Jurkats.

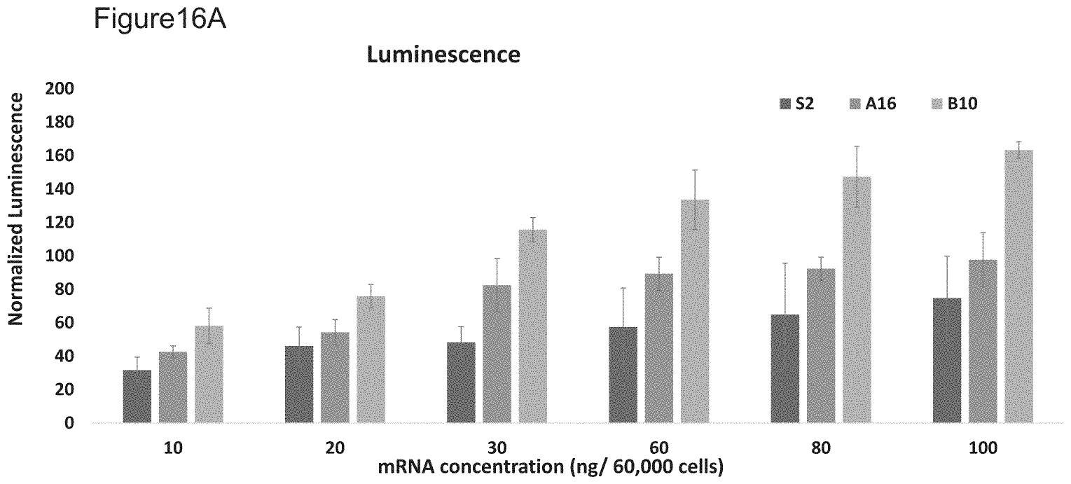

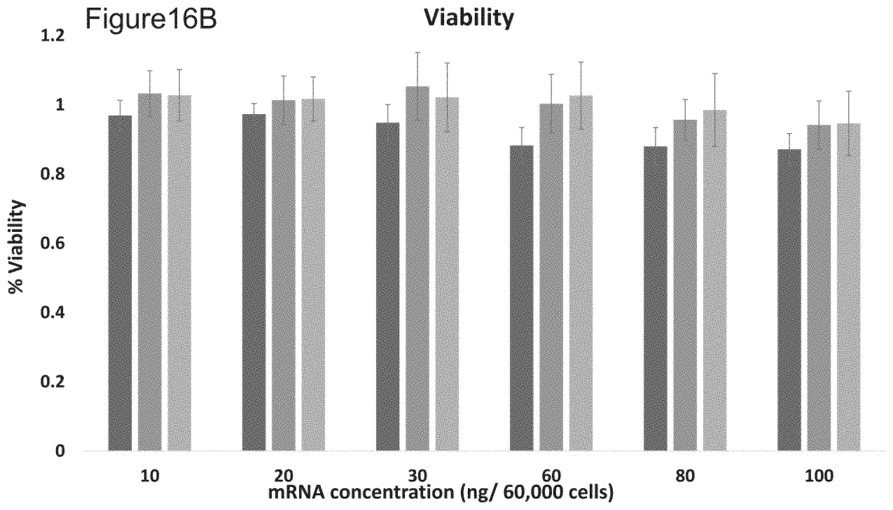

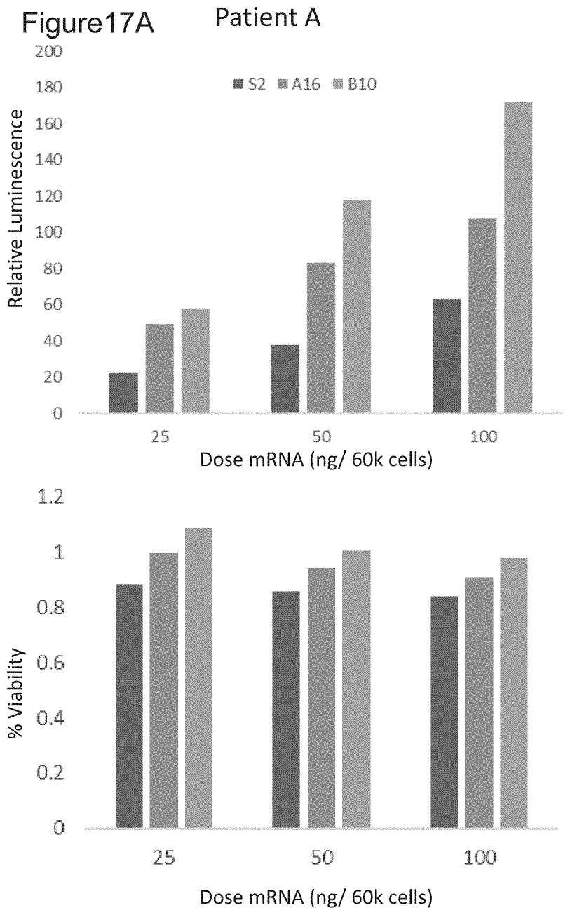

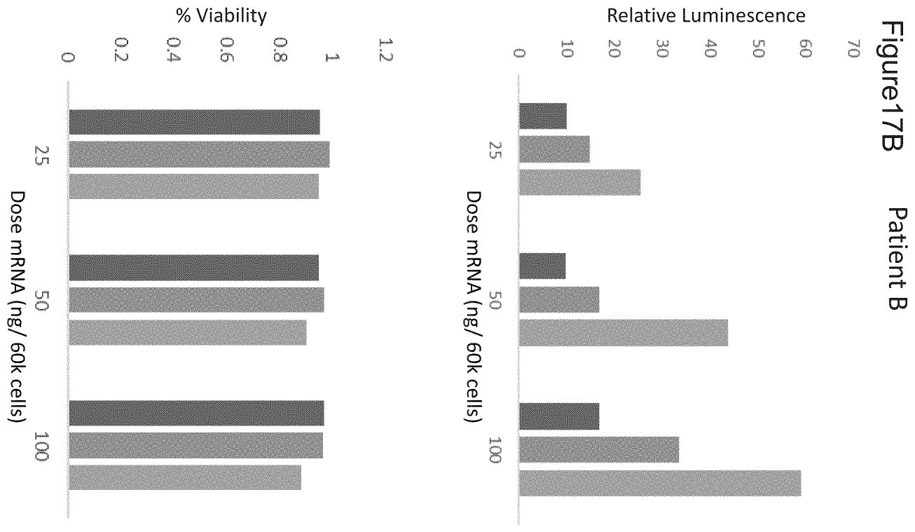

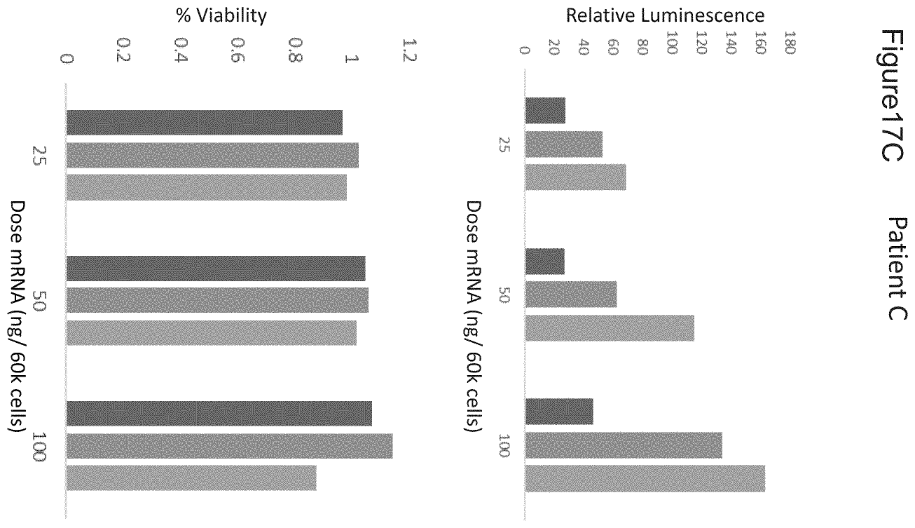

Additionally, Jurkats were treated for 24 hr with luciferase-encoding mRNA to assess the luminescence and viability for various representative formulations at different concentrations/doses (FIG. 16A and FIG. 16B). Furthermore, three different primary patient T cell samples were activated overnight and treated with doses of the standard, A16, or B10 formulation (FIG. 17A through FIG. 17C). Delivering luciferase encoding mRNA values were normalized to 0 ng treatment. Donor variability resulted in different overall luciferase readouts. Furthermore, additional studies are focused on the evaluation of top performing LNP (formulation B10) to deliver CAR mRNA to primary T cells.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A lipid nanoparticle (LNP) comprising at least one mRNA molecule and at least one compound or salt thereof having the structure of Formula (I) 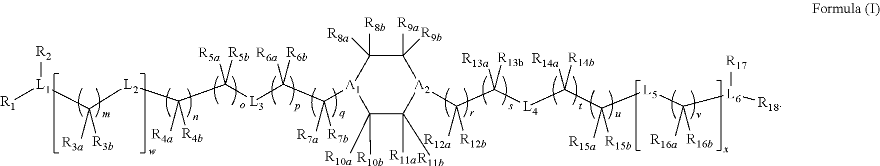
wherein A₁ and A₂ is independently selected from the group consisting of C, C(H), N, S, and P;
wherein each L₁, L₂, L₃, L₄, L₅, and L₆ is independently selected from the group consisting of C, C(H)₂, C(H)(R₁₉), O, N(H), and N(R₁₉);
wherein each R₁, R₂, R₃ₐ, R_{3b}, R₄ₐ, R_{4b}, R₅ₐ, R_{5b}, R₆ₐ, R_{6b}, R₇ₐ, R_{7b}, R₈ₐ, R_{8b}, R₉ₐ, R_{9b}, R₁₀ₐ, R_{10b}, R₁₁ₐ, R_{11b}, R₁₂ₐ, R_{12b}, R₁₃ₐ, R_{13b}, R₁₄ₐ, R_{14b}, R₁₅ₐ, R_{15b}, R₁₆, R₁₇, R₁₈, and R₁₉ is independently selected from the group consisting of H, halogen, alkyl, substituted alkyl, cycloalkyl, substituted cycloalkyl, —Y(R₂)_{z′}(R₂₁)_{z″}-cycloalkyl, substituted —Y(R₂₀)_{z′}(R₂₁)_{z″}-cycloalkyl, heterocycloalkyl, substituted heterocycloalkyl, —Y(R₂₀)_{z′}(R₂₁)_{z″}-heterocycloalkyl, substituted-(R₂₀)_{z′}(R₂₁)_{z″}-heterocycloalkyl, alkenyl, substituted alkenyl, cycloalkenyl, substituted cycloalkenyl, —Y(R₂₀)_{z′}(R₂₁)_{z″}-cycloalkenyl, substituted —Y(R₂₀)_{z′}(R₂₁)_{z″}-cycloalkenyl, alkynyl, substituted alkynyl, cycloalkynyl, substituted cycloalkynyl, —Y(R₂₀)_{z′}(R₂₁)_{z″}-cycloalkynyl, substituted —Y(R₂₀)_{z′}(R₂₁)_{z′}-cycloalkynyl, aryl, substituted aryl, —Y(R₂₀)_{z′}(R₂₁)_{z″}-aryl, substituted —Y(R₂₀)_{z′}(R₂₁)_{z″}-aryl, heteroaryl, substituted heteroaryl, —Y(R₂₀)z(R₂₁)z-heteroaryl, substituted —Y(R₂O)_{z′}(R₂₁)_{z″}-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R₂₀)_{z′}(R₂₁)_{z″}-ester, —Y(R₂₀)_{z′}(R₂₁)_{z″}, ═O, —NO₂, —CN, and sulfoxy;
wherein Y is selected from the group consisting of C, N, O, S, and P;
wherein each R₂₀ and R₂₁ is independently selected from the group consisting of H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, and sulfoxy;
wherein z′ and z″ are each independently an integer represented by 0, 1, or 2; and
wherein m, n, o, p, q, r, s, t, u, v, w, and x are each independently an integer represented by 0, 1, 2; 3, 4, or 5; and
wherein the mRNA molecule encodes a chimeric antigen receptor (CAR).

2. The LNP of claim 1, wherein the compound having the structure of Formula (I) is a compound having the structure selected from the group consisting of: 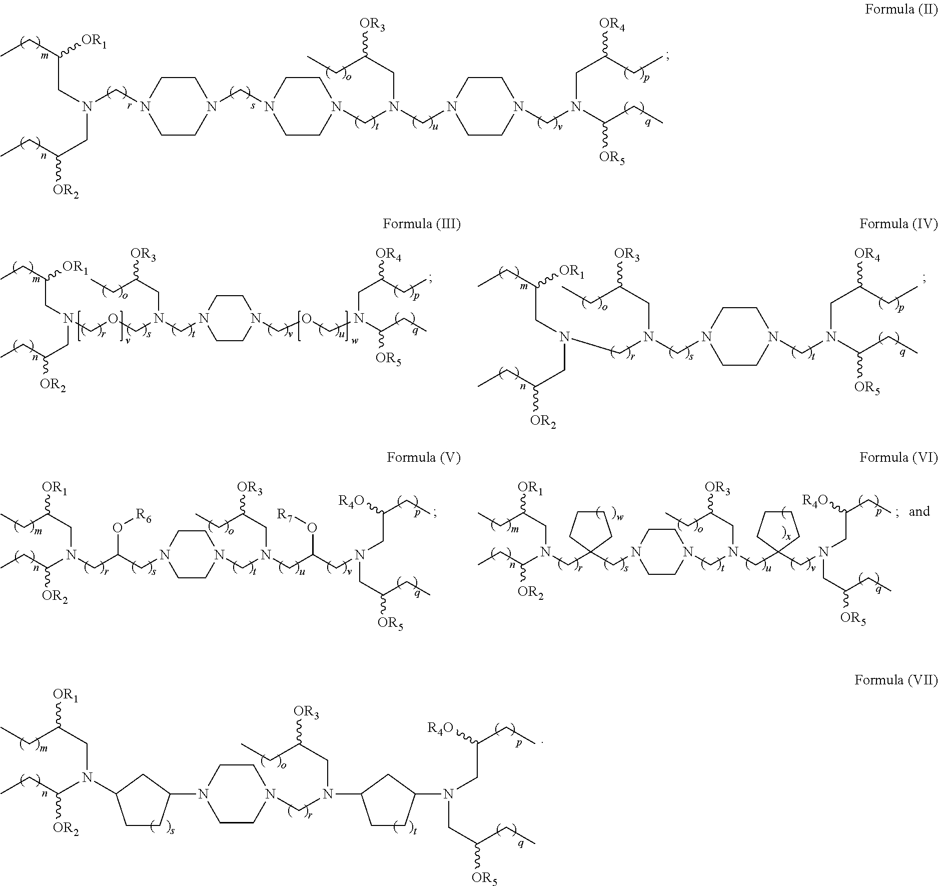
wherein each R₁, R₂, R₃, R₄, and R₅ is independently selected from the group consisting of H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, and ester,
wherein m, n, o, p, and q are each independently an integer from 0 to 25; and
wherein r, s, t, u, v, w, and x are each independently an integer represented by 0, 1, 2; 3, 4, and 5.

3. The LNP of claim 1, wherein the compound having the structure of Formula (I) is a compound having the structure selected from the group consisting of: 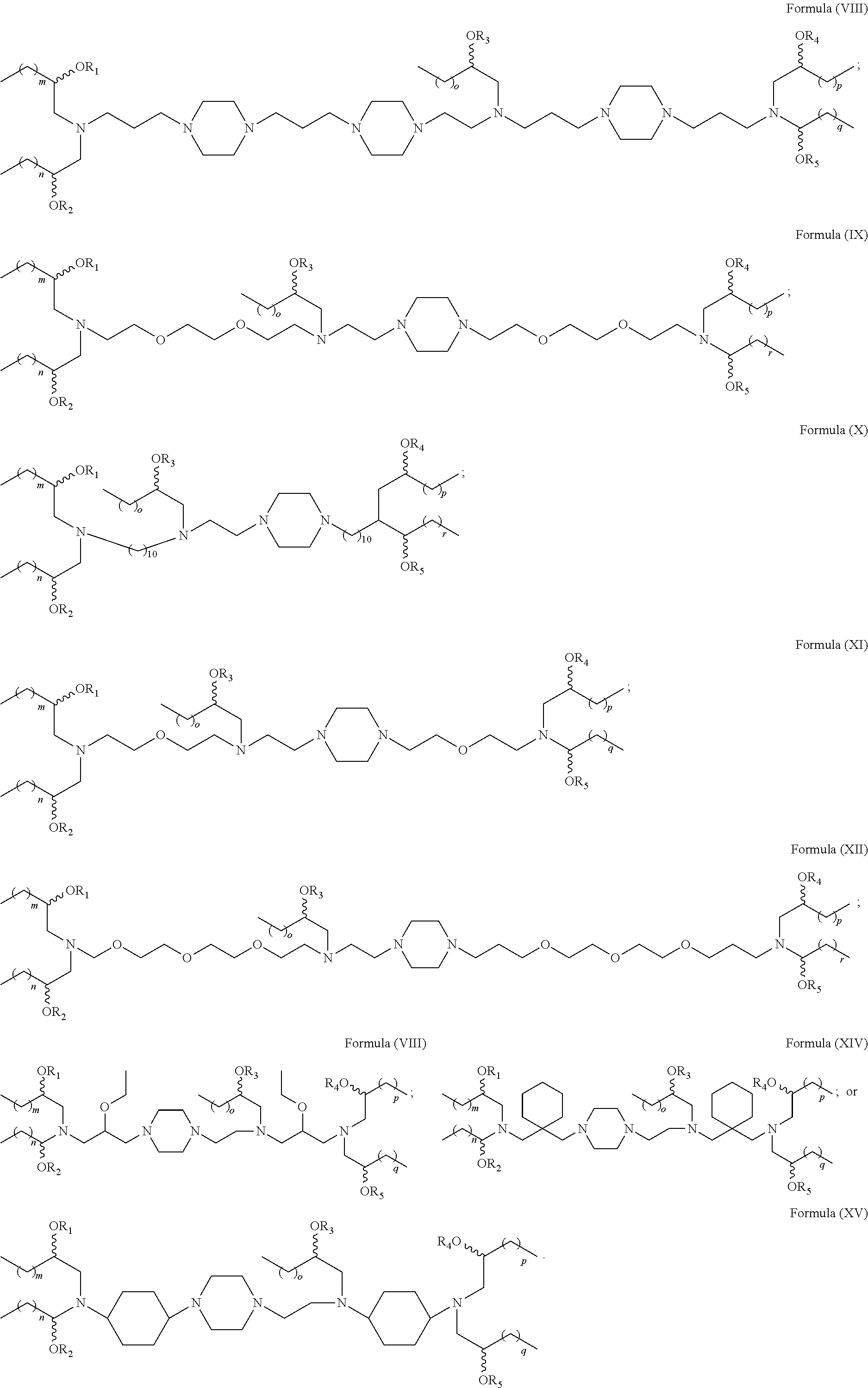
wherein each R₁, R₂, R₃, R₄, and R₅ is independently selected from the group consisting of H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, and ester; and
wherein m, n, o, p, and q are each independently an integer from 0 to 25.

4. The LNP of claim 1, wherein the compound having the structure of Formula (I) is an ionizable lipid.

5. The LNP of claim 1, wherein the mRNA molecule is encapsulated within the compound having the structure of Formula (I).

6. The LNP of claim 1, wherein the LNP comprises the compound or salt thereof having the structure of Formula (I) in a concentration range of about 1 mol % to about 100 mol %.

7. The LNP of claim 6, wherein the LNP comprises the compound or salt thereof having the structure of Formula (I) in a concentration range of about 10 mol % to about 50 mol %.

8. The LNP of claim 1, wherein the LNP further comprises at least one helper lipid.

9. The LNP of claim 8, wherein the LNP comprises at least one helper lipid in a concentration range of about 0.01 mol % to about 99.9 mol %.

10. The LNP of claim 9, wherein the LNP comprises at least one helper lipid in a concentration range of about 0.5 mol % to about 50 mol %.

11. The LNP of claim 8, wherein the helper lipid is selected from the group consisting of phospholipid, cholesterol lipid, polymer, and any combination thereof.

12. The LNP of claim 11, wherein the phospholipid is selected from the group consisting of dioleoyl-phosphatidylethanolamine (DOPE) or a derivative thereof, distearoylphosphatidylcholine (DSPC) or a derivative thereof, distearoyl-phosphatidylethanolamine (DSPE) or a derivative thereof, stearoyloleoylphosphatidylcholine (SOPC) or a derivative thereof, 1-stearioyl-2-oleoyl-phosphatidyethanol amine (SOPE) or a derivative thereof, N-(2,3-dioleoyloxy)propyl)-N,N,N-trimethylammonium chloride (DOTAP) or a derivative thereof, and any combination thereof.

13. The LNP of claim 11, wherein the LNP comprises a phospholipid in a concentration range of about 15 mol % to about 50 mol %.

14. The LNP of claim 11, wherein the cholesterol lipid is cholesterol or a derivative thereof.

15. The LNP of claim 11, wherein the LNP comprises a cholesterol lipid in a concentration range of about 20 mol % to about 50 mol %.

16. The LNP of claim 11, wherein the polymer is polyethylene glycol (PEG) or a derivative thereof.

17. The LNP of claim 11, wherein the LNP comprises a polymer in a concentration range of about 0.5 mol % to about 10 mol %.

18. The LNP of claim 1, wherein the LNP further comprises at least one selected from the group consisting of a nucleic acid molecule, adjuvant, therapeutic agent, and any combination thereof.

19. The LNP of claim 18, wherein the nucleic acid molecule is a therapeutic agent.

20. The LNP of claim 18, wherein the nucleic acid molecule is a DNA molecule or an RNA molecule.

21. The LNP composition of claim 18, wherein the nucleic acid molecule is selected from the group consisting of cDNA, mRNA, miRNA, siRNA, sgRNA, modified RNA, antagomir, antisense molecule, guide RNA molecule, CRISPR guide RNA molecule, peptide, therapeutic peptide, targeted nucleic acid, and any combination thereof.

22. The LNP of claim 21, wherein the mRNA encodes one or more antigens.

23. The LNP of claim 22, wherein the antigen comprises at least one selected from the group consisting of a viral antigen, a bacterial antigen, a fungal antigen, a parasitic antigen, an influenza antigen, a tumor-associated antigen, and a tumor-specific antigen.

24. The LNP of claim 18, wherein the nucleic acid molecule comprises a promoter or regulatory sequence.

25. The LNP of claim 18, wherein the mRNA molecule encoding a CAR, nucleic acid molecule, adjuvant, therapeutic agent, or any combination thereof is encapsulated within the compound having the structure of Formula (I).

26. A composition comprising at least one LNP of claim 1.

27. The composition of claim 26, wherein the composition is a vaccine.

28. A method of delivering at least one mRNA molecule encoding CAR to a subject in need thereof, comprising administering a therapeutically effectively amount of the LNP of claim 1 or a composition thereof to the subject,
wherein the LNP or the composition thereof delivers the mRNA molecule encoding CAR to a target.

29. The method of claim 28, wherein the target is selected from the group consisting of an immune cell, T cell, resident T cells, B cell, natural killer (NK) cell, cancerous cell, cell associated with a disease or disorder, tissue associated with a disease or disorder, brain tissue, central nervous system tissue, pulmonary tissue, apical surface tissue, epithelial cell, endothelial cell, liver tissue, intestine tissue, colon tissue, small intestine tissue, large intestine tissue, feces, bone marrow, macrophages, spleen tissue, muscles tissue, joint tissue, tumor cells, diseased tissues, lymph node tissue, lymphatic circulation, and any combination thereof.

30. The method of claim 28, wherein the method comprises a single administration of the LNP or the composition thereof.

31. The method of claim 28, wherein the method comprises multiple administrations of the LNP or the composition thereof.

32. The method of claim 28, wherein the LNP or the composition thereof is administered by a delivery route selected from the group consisting of intradermal, subcutaneous, intramuscular, intraventricular, intrathecal, oral delivery, intravenous, intratracheal, intraperitoneal, in utero delivery, and any combination thereof.

33. The method of claim 28, wherein the LNP or the composition thereof further comprises at least one nucleic acid molecule, adjuvant, therapeutic agent, or any combination thereof.

34. The method of claim 33, wherein the nucleic acid molecule is a therapeutic agent.

35. The method of claim 33, wherein the nucleic acid molecule is a DNA molecule or an RNA molecule.

36. The method of claim 33, wherein the nucleic acid molecule is selected from the group consisting of cDNA, mRNA, miRNA, siRNA, sgRNA, antagomir, antisense molecule, guide RNA molecule, CRISPR guide RNA molecule, peptide, therapeutic peptide, targeted nucleic acid, and any combination thereof.

37. The method of claim 36, wherein the mRNA encodes one or more antigens.

38. The method of claim 37, wherein the antigen comprises at least one selected from the group consisting of a viral antigen, a bacterial antigen, a fungal antigen, a parasitic antigen, an influenza antigen, a tumor-associated antigen, and a tumor-specific antigen.

39. The method of claim 38, wherein the method treats or prevents at least one selected from the group consisting of a viral infection, a bacterial infections, a fungal infection, a parasitic infection, influenza infection, cancer, arthritis, heart disease, cardiovascular disease, neurological disorder or disease, genetic disease, autoimmune disease, fetal disease, genetic disease affecting fetal development, and any combination thereof.

40. The method of claim 33, wherein the nucleic acid molecule comprises a promoter or regulatory sequence.

41. The method of claim 33, wherein the mRNA encoding a CAR, nucleic acid molecule, adjuvant, therapeutic agent, or any combination thereof is encapsulated within the compound having the structure of Formula (I).

42. The method of claim 28, wherein the LNP composition is a vaccine.

43. A method of delivering at least one mRNA molecule encoding CAR and at least one nucleic acid molecule, adjuvant, therapeutic agent, or any combination thereof to a subject in need thereof, comprising administering a therapeutically effectively amount of the LNP of claim 18 or a composition thereof to the subject,
wherein the LNP or the composition thereof delivers the mRNA molecule and the nucleic acid molecule, adjuvant, therapeutic agent, or any combination thereof to a target.

44. The method of claim 43, wherein the target is selected from the group consisting of an immune cell, T cell, resident T cells, B cell, natural killer (NK) cell, cancerous cell, cell associated with a disease or disorder, tissue associated with a disease or disorder, brain tissue, central nervous system tissue, pulmonary tissue, apical surface tissue, epithelial cell, endothelial cell, liver tissue, intestine tissue, colon tissue, small intestine tissue, large intestine tissue, feces, bone marrow, macrophages, spleen tissue, muscles tissue, joint tissue, tumor cells, diseased tissues, lymph node tissue, lymphatic circulation, and any combination thereof.

45. The method of claim 43, wherein the method is a gene delivery method.

46. A method of preventing or treating a disease or disorder in a subject in need thereof, comprising administering a therapeutically effectively amount of the LNP of claim 1 or a composition thereof to the subject.
